(19) United States
(12) Patent Application Publication
(10) Pub. No.: US 2011/0064681 A1
(43) Pub. Date: Mar. 17, 2011

(54) POLYURETHANES AS RHEOLOGICAL MODIFYING MEANS FOR COSMETIC PREPARATIONS

(75) Inventors: Volker Wendel, Seeheim-Jugenheim (DE); Helmuth Völlmar, Ludwigshafen (DE); Holger Türk, Mannheim (DE); Markus Buchmann, Freinsheim (DE); Valerie Andre, Ludwigshafen (DE); Matthias Laubender, Shifferstadt (DE); Claudia Wood, Weinheim (DE); Andree Dragon, Speyer (DE)

(73) Assignee: BASF SE, Ludwigshafen (DE)

(21) Appl. No.: 12/991,009

(22) PCT Filed: May 6, 2009
(86) PCT No.: PCT/EP09/55440

§ 371 Date: Nov. 4, 2010

(30) Foreign Application Priority Data
May 6, 2008 (EP) 08155675.5
Mar. 25, 2009 (EP) 09156217.3

(51) Int. Cl. A61K 8/87; A61Q 1/00; A61Q 15/00; A61Q 19/00

(52) U.S. Cl. 424/59; 424/68; 424/70.11; 514/625

(57) ABSTRACT

The present invention relates to cosmetic preparations comprising novel polyurethanes or mixtures thereof as means for modifying rheological properties.

The present invention relates to cosmetic preparations which comprise new types of polyurethanes (PU) or mixtures of such polyurethanes PU, in particular as means for modifying the rheological properties.

In particular, the invention relates to cosmetic preparations comprising a water-dispersible polyurethane (PU) with an essentially linear backbone composed of alternating hydrophilic and hydrophobic sections, where
a. the two terminal sections (T) are hydrophobic,
b. in each case one hydrophilic section (S) directly adjoins each section T,
c. at least one hydrophobic section (D) directly adjoins each section S on at least one side, and
d. where at least one hydrophilic section (P) is present, where at least one hydrophobic section D separates two sections P if more than one section P is present,
   and the polyurethane comprises at least three hydrophilic sections, and the ratio of the molecular weights of each hydrophilic section S to the molecular weight of each hydrophilic section P is from 1:1.4 to 1:140, the at least two hydrophobic sections D are aliphatic diisocyanate radicals and the at least one hydrophilic section P is a polyether radical with a molecular weight of at least 1500 g/mol.

The polyurethanes present in the cosmetic preparations according to the invention are polymers which are formed by reacting alcohol alkoxylates and/or polyether polyols with isocyanates or polyisocyanates, and are also termed polyether urethanes hereinbelow. The abbreviation “PU” is also used hereinbelow for the polyurethanes present in the cosmetic preparations according to the invention.

Cosmetics can encompass all measures which, for esthetic reasons, make changes to skin and hair or are used for cleaning the body. Cosmetics means in particular to care for, to improve and to beautify the body exterior in order to please, in a visible, feelable and smellable way, both those around us and also ourselves.

Within the context of these inventions, cosmetic preparations are, however, also understood as meaning those preparations which serve for oral hygiene and cosmetics.

Within the context of this invention, cosmetic preparations are also understood as meaning dermatological preparations.

Modifying the rheological properties is very generally understood as meaning the change in the shaping and flow behavior of material. The most important rheological properties are viscosity, thixotropy, structural viscosity, rheopexy and dilatancy. These terms are known to the person skilled in the art.

Modifying the rheology is understood in particular as meaning the increase in the viscosity of liquids, usually also referred to as “thickening”. This viscosity increase can range to the formation of gels or solids. Water-dispersible polyurethanes which lead to an increase in viscosity and thus to an effect as thickeners are known.

Thickeners that are usually used are fatty acid polyethylene glycol monoesters, fatty acid polyethylene glycol diesters, fatty acid alkanolamides, oxethylated fatty alcohols, ethoxylated glycerol fatty acid esters, cellulose ethers, sodium alginate, polyacrylic acids (INCI: Carbomer, for example Carbopol® grades), taurate derivatives, polysaccharides and neutral salts such as, for example, sodium chloride.

However, depending on the preparation to be thickened, the use of the aforementioned customary thickener is associated with disadvantages. For example, the thickening effect and the salt stability of the thickener may be unsatisfactory and hinder its incorporation into the preparation to be thickened. It is known that thickeners such as e.g. crosslinked (hydrophobically modified) polyacrylic acids in the neutralized state react very sensitively to salt or surfactant or a mixture thereof. Thus, the addition of salt can lead to abrupt and drastic viscosity reduction. Consequently, it is for example unusual to use such polymers in shampoo formulations as thickeners. On account of the salt concentrations present therein (surfactants, surfactant mixtures, NaCl as impurity in surfactants), no significant viscosity increase can be brought about by adding customary thickeners. The presence of cationic auxiliaries may even lead to complex formation and precipitate. In the field of cosmetic preparations, the search for salt-tolerant (salt-stable) thickeners which, coupled with good thickening power in the presence of salt, also lead to preparations with a good texture and pleasant feel on the skin and/or the hair has proven extremely difficult.

Essential requirements of thickeners for cosmetic preparations are moreover the compatibility with the numerous further ingredients of these preparations, in particular with salts and surfactants, and also the trouble-free incorporability.

Even upon long-term storage over several weeks to months, temperature and pH changes, the thickened preparations must have no essential changes in rheology, physical and chemical quality. Lastly, it should be possible to produce these thickeners cost-effectively and without noticeable environmental impact.

As early as the end of the 1970s, thickeners of the so-called HEUR type were described in U.S. Pat. No. 4,079,028 (the acronym HEUR is derived from “nonionic hydrophobically modified ethylene oxide urethane block copolymer”). These thickeners are composed of linear and/or branched polyethylene glycol blocks and hydrophobic segments which are generally linked together via urethane groups (using amines instead of alcohols results in urea groups). For some time, such HEUR thickeners have already been used in diverse fields of application for thickening water-based emulsion paints. The principle of action of the thickening effect of the HEUR thickeners is assumed to be that the polyethylene glycol segments ensure the compatibility with water and the hydrophobic segments, via an association with one another and with dispersed binder droplets of the emulsion paint to be thickened, build up in said paint a viscosity-conferring three-dimensional molecular association. Preferred hydrophobic building blocks in standard commercial HEUR thickeners are relatively long-chain, usually monofunctional alcohols, such as, for example, n-octanol, n-dodecanol, isotridecyl alcohol, isononylphenol or methyl ricinoleate. These alcohols are used predominantly as such, but also in the form of their addition products with a few equivalents of ethylene oxide.

In the field of cosmetics, the HEUR types to be mentioned are particularly the Aculyn™ grades (Aculyn™ 44 and Aculyn™-46, Rohm & Haas).

U.S. Pat. No. 4,079,028 and U.S. Pat. No. 4,155,892 disclose linear polyurethane thickeners and their use in cosmetics. The production of these polyurethane thickeners takes place in the presence of tin-containing polymerization catalysts.

EP 1013264-B and EP 1584331-A disclose cosmetic preparations which comprise polyurethane thickeners and mono- or polyhydric lower alcohols. The polyurethane thickeners are produced without catalysts in a single-step process by reaction without a diluent from polyol, polyisocyanate and fatty alcohol, which may, if desired, be ethoxylated. The viscosity of a preparation which these thickeners comprise allegedly does not change when the salt concentration in the preparation changes.

EP 1241198-A describes water-soluble or water-dispersible polyurethanes, obtained in a single- or multi-stage reaction, while maintaining a NCO/OH equivalent ratio of from 0.5:1 to 1.2:1 as reaction products from

A) a mixture of at least one polyether polyol a1) of average functionality ≧3 and at least one urethane-group-containing polyether polyol a2) of average functionality ≧4,
B) at least one monoalcohol having 6 to 22 carbon atoms,
C) at least one (cyclo)aliphatic and/or aromatic diisocyanate,
D) optionally at least one monoisocyanate having 4 to 18 carbon atoms and
E) optionally at least one polyisocyanate of average functionality >2.

WO 02/44236 describes cosmetic preparations comprising polyurethane thickeners of the formula R1(CH2CH2O)n1CONH—X—NHCOO(CH2CH2O)mCONH—Y—NH—OC(OCH2CH2)n2OR2, in which R1 and R2, independently of one another, are linear or branched, saturated or unsaturated alkyl radicals having 6 to 22 carbon atoms and 0 and/or 1 to 3 double bonds, n1 and n2 in total are 0 or numbers from 1 to 100, m is numbers from 4 to 500, —(CH2)z1-CR3R4]a1-[Ph]x-[CR5R6-(CH2)z2]a2, in which R3, R4, R5 and R6, independently of one another, are hydrogen or alkyl radicals having 1 to 4 carbon atoms, Ph is an optionally alkyl-substituted phenyl radical and x, a1, a2, z1 and z2, independently of one another, are 0 or 1.

WO 02/83093 describes cosmetic preparations comprising polyether urethane thickeners to formula R1-(OCH2CH2)m-[CO—NH—CH2-CH2-CH2-CH2-CH2-CH2-NH—CO]x-(CH2CH2O)n-R2, in which R1 and R2, independently of one another, are linear or branched alkyl and/or alkenyl radicals having 6 to 22 carbon atoms, x is numbers from 1 to 3 and m and n, independently of one another, are numbers from 10 to 100. Polyurethane thickeners for cosmetic preparations.

WO 2006/002 813 A discloses polyurethane thickeners for various applications in aqueous media. These thickeners are prepared from hydrophilic polyols with at least two hydroxy groups, one or more hydrophobic compounds, e.g. long-chain alcohols and at least difunctional isocyanates. Here, an excess of NCO groups is used. The catalyst used in the preparation may be tin-containing, zinc-containing or an amine.

EP 0 725 097 B discloses polyurethane thickeners, in the preparation of which polyethers, produced by alkoxylation of alcohols or alkylphenols, are reacted with polyisocyanates, where the ratio of NCO to OH equivalents is in the range from 0.9:1 to 1.2:1. These thickeners are proposed for use in the field of low shear forces, e.g. for the flow of aqueous emulsion paints.

It was an object of the present invention to provide cosmetic preparations of increased viscosity, the rheological properties of which essentially do not change at low and high polyelectrolyte concentrations, pH or temperature fluctuations over periods of several weeks. The cosmetic preparations, in particular emulsions and dispersions, should be stable with regard to their chemical and physical properties. The cosmetic preparations should convey a soft, nongreasy and nonsticky feel to the touch. Furthermore, the cosmetic preparations should be as cosmetically and dermatologically acceptable as possible, in particular they should be tin-free.

The aforementioned objects were achieved through the provision of a cosmetic preparation comprising a water-dispersible polyurethane (PU) with an essentially linear backbone composed of alternating hydrophilic and hydrophobic sections, where
a. the two terminal sections (T) are hydrophobic,
b. in each case one hydrophilic section (S) directly adjoins each section T,
c. at least one hydrophobic section (D) directly adjoins each section S on at least one side, and
d. where at least one hydrophilic section (P) is present, where at least one hydrophobic section D separates two sections P if more than one section P is present,
   and the polyurethane comprises at least three hydrophilic sections, and the ratio of the molecular weights of each hydrophilic section S to the molecular weight of each hydrophilic section P is from 1:1.4 to 1:140, the at least two hydrophobic sections D are aliphatic diisocyanate radicals and the at least one hydrophilic section P is a polyether radical with a number-average molecular weight of at least 1500 g/mol.

According to the invention, the polyurethanes are dispersible in water. According to the invention, “dispersible in water” also includes the polyurethanes being emulsifiable or completely or partially soluble in water.

Preferably, the polyurethanes PU used in the preparations according to the invention have the property that, in a dispersion in water at concentrations between 0.1 and 10 g/l, they form micelles with an average particle size of less than or equal to 200 nm, in particular less than or equal to 100 nm (can be determined by means of dynamic light scattering as described below). It is therefore also possible to talk of nanodispersible polyurethanes. The critical substance concentration for micelle formation, also critical micelle concentration (CMC) is accordingly preferably less than 0.1 g/l.

The polyurethanes used in the preparations according to the invention have an essentially linear backbone, i.e. they have no branching points or few branching points relative to the overall length. Branches therefrom may be present in hydrophobic and/or hydrophilic sections.

The polyurethanes PU used in the preparations according to the invention are neither star-shaped nor crosslinked. Polyurethanes of this type and the preparation thereof are known from the prior art and are not part of this invention.

Preferably, the polyurethanes used in the preparations according to the invention have less than or equal to 4 branches per molecule, particularly preferably less than or equal to 3 branches per molecule. In a particularly preferred embodiment, the polyurethanes used in the preparations according to the invention have no branches outside of the edge-position sections T. Methods for determining branching such as e.g. via NMR spectroscopy are known to the person skilled in the art.

The backbone of the polyurethanes used in the preparations according to the invention is composed of alternating hydrophobic and hydrophilic sections, where although the hydrophobic and hydrophilic sections alternate in the sequence, they may be different in their size, length and nature. A hydrophilic section directly adjoins on both sides a hydrophobic section. These hydrophobic sections can be, independently of one another, identical or different. Each section may be short-chain or an oligomer radical or a polymer radical.

Hydrophilic refers here to those sections which exhibit marked interaction with water. In general, hydrophilic sections consist of radicals of substances which are themselves hydrophilic.

Typical hydrophilic groups known to the person skilled in the art are nonionic polyether radicals. Preferred polyether radicals essentially comprise unbranched alkylene oxide radicals.

Polyether radicals can be homo-alkylene oxide radicals, or comprise mixtures of different alkylene oxide radicals. These different alkylene oxide radicals can be present in the polyether radicals in random distribution or be present in block form. Preferred polyether radicals are homo-ethylene oxide radicals or homo-propylene oxide radicals. According to another embodiment, the polyether radicals comprise mixtures of ethylene oxide radicals and propylene oxide radicals. These may be present in the polyether radicals in random distribution or may be present in block form.

A particularly preferred embodiment covers polyether radicals which have at least 50% by weight of ethylene oxide radicals, for example polyether radicals, which have more than 50% by weight of ethylene oxide radicals and propylene oxide radicals as further alkylene oxide radicals. The polyether radicals very particularly preferably consist of ethylene oxide radicals.

The hydrophilicity of a substance can be determined for example by an opacity measurement of an aqueous solution.

The hydrophobic sections present in the polyurethanes used in the preparations according to the invention behave oppositely toward water compared with the hydrophilic sections. In general, the hydrophobic sections consist of radicals of substances which are immiscible or only very poorly miscible with water and are virtually always lipophilic, i.e. they readily dissolve in nonpolar solvents, fats and oils. Typical hydrophobic groups are, for example, hydrocarbon radicals, in particular long-chain hydrocarbon radicals. According to the invention, unbranched or slightly branched hydrocarbon radicals are preferred. According to one of the embodiments, the hydrocarbon radicals are unbranched. Long-chain aliphatic alcohols, aromatic alcohols and also aliphatic diisocyanates are examples of hydrophobic substances whose radicals may be present in the hydrophobic sections of the polyurethanes used in the preparations according to the invention.

A molecule which has both hydrophobic and hydrophilic sections is generally referred to as an amphiphilic molecule. Examples which may be mentioned are inter alia phospholipids, emulsifiers and surfactants. A measure of the hydrophilicity of an amphiphilic compound is the HLB value. The HLB value (hydrophilic-lipophilic balance) describes the hydrophilic and lipophilic fraction of primarily nonionic surfactants and was proposed in the 20th century by W. C. Griffin (Griffin, W. C.: Classification of surface active agents by HLB, J. Soc. Cosmet. Chem. 1, 1949). The HLB value can be calculated as follows (see formula I):

$$HLB = 20 * ( 1 - \frac{{M}_{1}}{M} ) \qquad ( formula I )$$

where MI is the molar mass of the hydrobic fraction of a molecule and M is the molar mass of the entire molecule. The factor 20 is a scaling factor freely selected by Griffin. It therefore generally gives rise to a scale from 1 to 20. A HLB value of 1 indicates a lipophilic compound; a chemical compound with a HLB value of 20 has a high hydrophilic fraction.

The polyurethanes used in the preparations according to the invention preferably have a HLB value according to Griffin of greater than or equal to 7, particularly preferably of greater than or equal to 14, on a scale from 1 to 20.

Polyurethanes used in the preparations according to the invention comprise at least two terminal hydrophobic sections (T). The polyurethanes PU used in the preparations according to the invention can be branched to a low degree in the molecule interior (if desired by using tri- or polyisocyanates in low fractions), meaning that then more than two terminal hydrophobic sections T could be present.

Preferably, the polyurethanes PU used in the preparations according to the invention in the molecule interior are unbranched and comprise two terminal hydrophobic sections T. Their end position means that they directly adjoin only one further section of the polyurethanes used in the preparations according to the invention.

The terminal sections T can be identical or, independently of one another, different. The terminal hydrophobic sections T may be branched or unbranched. Preferably, at least one of the two terminal hydrophobic sections T of the polyurethanes PU used in the preparations according to the invention is branched.

Preferably, the terminal hydrophobic sections T comprise a chain of carbon atoms. Preferably, the chain length of the sections T is in the range from 4 to 30 carbon atoms, particularly preferably in the range from 6 to 26 and very particularly preferably in the range from 8 to 20 carbon atoms.

Such sections T can consist for example of aromatic radicals, but also of alkyl radicals. Thus, the sections T may be branched or unbranched alkyl radicals, or comprise these. Preferably, at least one section T is a branched alkyl radical. Branched means that branches attach to one or more carbon atoms of the alkyl radical. Usually, a branching of an alkyl means that, besides the members of the main chain, one or more additional carbon atoms are covalently bonded in one or two positions to a carbon atom of the carbon backbone and form a side chain. The side chains can have identical or different sizes. Preferably, the side chains are themselves alkyl radicals or alkylene radicals, particularly preferably alkyl radicals, in particular unbranched alkyl radicals.

In one embodiment, the side chains of the alkyl radicals preferably have a chain length of not more than 6 carbon atoms. In another embodiment, the branches are preferably considerably shorter chains than the main chain. Preferably, each branch of sections T of the polyurethanes used in the preparations according to the invention has at most a chain length which corresponds to half of the chain length of the main chain of this section T. The branched alkyl radicals are particularly preferably iso- and/or neo-alkyl radicals. Preferably, the chain length of the main chain of alkyl radicals which are present in sections T is in the range from 4 to 30 carbon atoms, for example alkyl radicals of butane, pentane, hexane, heptane, octane, nonane, decane, undecane, dodecane, tridecane, tetradecane, pentadecane, hexadecane, heptadecane, octadecane, nonadecane, icosane, henicosane, docosane, tricosane, tetracosane, pentacosane, hexacosane, heptacosane, octacosane, nonacosane and/or triacontane. Branched alkyl radicals of these alkanes can be used. Radicals of cycloalkanes or alkenes may also likewise be present. The sections T particularly preferably comprise alkyl radicals with a number of carbon atoms in the range from 6 to 26, for example radicals of hexane, heptane, octane, nonane, decane, undecane, dodecane, tridecane, tetradecane, pentadecane, hexadecane, heptadecane, octadecane, nonadecane, icosane, henicosane, docosane, tricosane, tetracosane, pentacosane and/or hexacosane, and very particularly preferably in the range from 8 to 20 carbon atoms, for example radicals of octane, nonane, decane, undecane, dodecane, tridecane, tetradecane, pentadecane, hexadecane, heptadecane, octadecane, nonadecane and/or icosane. Branched alkyl radicals of these alkanes can be used just as much as radicals of cyclolalkanes or alkenes.

In one preferred embodiment, the branched alkyl radicals used are radicals of iso-alkanes. Particular preference is given to a C13-alkyl radical, in particular an iso-C13-alkyl radical.

The introduction of the sections T into the polyurethanes used in the preparations according to the invention can take place in various ways, for example as part of ethoxylated fatty alcohols.

Cosmetic preparations comprising mixtures of the polyurethanes PU described above, the terminal, hydrophobic sections T of which are branched and/or unbranched alkyl radicals, are also in accordance with the invention. Of suitability for use in the cosmetic preparations are also mixtures in which the polyurethanes PU described above are present which have both branched and unbranched terminal, hydrophobic sections T.

A hydrophilic section (S) is present directly adjacent to each section T in polyurethanes used in the preparations according to the invention. The section S has a distancing effect as a so-called spacer S. A certain spatial flexibility of the sections S is desired. Preferably, the hydrophilic sections are unbranched.

In the polyurethanes PU used in the preparations according to the invention, the spacers S may be identical or, independently of one another, different. In one embodiment, the hydrophilic sections S are of different length and linear.

In a further preferred embodiment, the sections S of the polyurethanes used in the preparations according to the invention have a chain length of from 5 to 100 atoms, preferably from 6 to 90 atoms and particularly from 8 to 80 atoms, in particular chains from 15 to 60 atoms.

The sections S can comprise radicals of alkylene oxides. Preferably, the number is in the range from 2 to 30 alkylene oxide radicals, particularly preferably in the range from 3 to 25 alkylene oxide radicals and very particularly preferably in the range from 3 to 20 alkylene oxide radicals.

The at least two hydrophilic sections S of the polyurethanes can in each case be ethylene oxide radicals. In one preferred embodiment, the hydrophilic sections S comprise ethylene oxide radicals, the number of which is in the range from 2 to 30 radicals, particularly preferably in the range from 3 to 25 ethylene oxide radicals and very particularly preferably in the range from 3 to 20 radicals.

Mixtures of ethylene oxide radicals and propylene oxide radicals or only propylene oxide radicals in the sections S are also possible.

The sections S can likewise comprise relatively long-chain alkylene oxides, although it must be ensured that the sections S overall must be hydrophilic (e.g. by virtue of a correspondingly high ethylene oxide fraction).

At least one hydrophobic section (D) adjoins each hydrophilic section S directly on at least one side. Here, a section S may also be present in the molecule interior of the polyurethanes used in the preparations according to the invention. In this case, this section S is not bonded like an edge-position section S to a section D and a section T, but to sections D on at least two sides. Preferably, a section A in the molecule interior is bonded to in each case one section D on both sides. For all edge-position sections S, it is the case that they are bonded directly to a terminal section T. Should a section S be branched to a low degree, then it could be directly bonded to hydrophobic sections D at two or more points. Preferably, in each case one hydrophobic section D adjoins each linear hydrophobic spacer S on one or two sides. In a particularly preferred embodiment, all, i.e. in particular the two sections S are unbranched, edge-position, and joined to a section T on the one side and a section D on the other side.

The polyurethanes used in the preparations according to the invention comprise at least two hydrophobic sections D. The hydrophobic sections D can be identical or, independently of one another, different.

The sections D can be branched with short-chain hydrophobic branches or be unbranched. Preferably, the sections D are unbranched.

Preferably, the sections D comprise a hydrophobic chain of carbon atoms, the length of which is the range from 2 to 20 carbon atoms, preferably 3 to 16 carbon atoms and in particular in the range from 4 to 12 carbon atoms.

Preferably, the sections D comprise diisocyanate radicals. The sections D particularly preferably comprise radicals of aliphatic diisocyanates. Thus, for example, a hydrophobic section D can consist of one or more aliphatic diisocyanate radicals. Preferably, a section D consists of one to ten aliphatic diisocyanate radicals, particularly preferably of one up to five aliphatic diisocyanate radicals, and it very particularly preferably comprises one, two or three aliphatic diisocyanate radicals. The hydrophobic sections D can comprise aliphatic diisocyanate radicals with long, medium-length or short aliphatic units.

In one of the preferred embodiments, the sections D of the polyurethanes used in the preparations according to the invention are cycloaliphatic or aliphatic diisocyanate radicals. The sections D are particularly preferably aliphatic diisocyanate radicals.

Aliphatic diisocyanates which may be mentioned by way of example are: 1,4-butylene diisocyanate, 1,12-dodecamethylene diisocyanate, 1,10-decamethylene diisocyanate, 2-butyl-2-ethylpentamethylene diisocyanate, 2,4,4- or 2,2,4-trimethylhexamethylene diisocyanate and in particular hexamethylene diisocyanate (HDI).

Cycloaliphatic diisocyanates which may be mentioned by way of example are: isophorone diisocyanate (IPDI), 2-isocyanatopropylcyclohexyl isocyanate, 4-methyl-cyclohexane 1,3-diisocyanate (H-TDI) and 1,3-bis(isocyanatomethyl)cyclohexane. Also so-called H₁₂-MDI or diisocyanates termed “saturated MDI”, such as e.g. 4,4′-methylenebis(cyclohexyl isocyanate) (alternatively also called dicyclohexylmethane 4,4′-diisocyanate) or 2,4′-methylenebis(cyclohexyl) diisocyanate may be present as radicals in sections D of the polyurethanes PU used in the preparations according to the invention.

It is of course possible to use mixtures of the aforementioned diisocyanates in order to prepare mixtures of different polyurethanes PU used in the preparations according to the invention.

The polyurethanes used in the preparations according to the invention comprise at least one hydrophilic section (P). Here, it is the case that at least one hydrophobic section D directly adjoins P on at least one side. The sections P of the polyurethanes used in the preparations according to the invention may be identical or, independently of one another, different.

If more than one section P is present in a polyurethane used in the preparations according to the invention, then there is at least one hydrophobic section D between the hydrophilic sections P. In one embodiment, the polyurethanes used in the preparations according to the invention can comprise between two hydrophilic sections P a sequence of sections in the order hydrophobic section D, then hydrophilic section S, then again hydrophobic section D. Thus, if more than one section P is present in a polyurethane used in the preparations according to the invention, then in such a case the sections in the molecule interior can have a sequence P-D-P or P-D-S-D-P. Should more than two sections P be present, then both sequences in one molecule are possible.

Preferably, only one or two sections P are present in a molecule of the polyurethanes used in the preparations according to the invention.

Preferably, the hydrophilic sections P are essentially linear polyether radicals, e.g. polyalkylene oxides. The hydrophilic sections P are particularly preferably radicals of polyetherdiols, in particular of polyethylene glycols. The at least one hydrophilic section P of the polyurethanes used in the preparations according to the invention is preferably composed of polyethylene oxide.

According to the invention, the essentially linear polyether radicals which form the sections P have a number-average molecular weight of at least 1500 g/mol. In general, the sections P have molecular weights of average size, e.g. up to 20 000 g/mol.

In a particularly preferred embodiment, the essentially linear polyether radicals have number-average molecular weights in the range from 1500 g/mol to 12 000 g/mol. Particularly preferably, the molecular weight of the sections P is less than or equal to 10 000 g/mol and particularly preferably in the range from 4000 g/mol to 9000 g/mol. The linear polyether radicals very particularly preferably have molecular weights of greater than or equal to 6000 g/mol.

All of the hydrophilic sections of the polyurethanes used in the preparations according to the invention, i.e. both sections and also sections P, may be polyether radicals.

In a preferred embodiment, the hydrophilic sections of the polyurethanes used in the preparations according to the invention consist of
polyalkylene oxide units (sections P) and
polyethylene oxide units (sections S).

In a particularly preferred embodiment of the PU used in the preparations according to the invention, all of the sections P and S consist of polyethylene oxide units.

The backbone of the polyurethanes used in the preparations according to the invention comprises essentially radicals of polyethers and diisocyanates.

The polyurethanes used in the preparations according to the invention comprise at least three hydrophilic sections. In one of the preferred embodiments, these are two sections S and at least one section P.

In a particularly preferred embodiment, the sequence of the sections of the polyurethanes used in the preparations according to the invention is either T-S-D-P-D-S-T or T-S-D-P-D-P-D-S-T.

For each section P, it is the case that its size is larger relative to the size of any spacer S present in the same molecule.

The ratio of the molecular weights of each hydrophilic section S of the polyurethanes used in the preparations according to the invention to the molecular weight of each hydrophilic section P is in the range from 1:1.4 to 1:140, preferably in the range from 1:1.7 to 1:120. In a preferred embodiment, the ratio is 1:x, where x is equal to or greater than 2, preferably equal to or greater than 2.3 and particularly preferably x is equal to or greater than 2.8. The ratio is particularly preferably in the range from 1:2.8 to 1:115, very particularly preferably in the range from 1:3 to 1:95 and especially preferably in the range from 1:3.4 to 1:80.

Likewise in accordance with the invention are cosmetic preparations which comprise polyurethanes PU as described above, for which it is additionally the case that they are a mixture. Such a mixture can comprise e.g. polyurethanes which do have the same sequence of the sections T, S, D and/or P, but differ from one another structurally in at least one of the sections. One example of this which may be mentioned is a different section composition or a different section chain length. Thus, in a mixture of polyurethanes PU, sections T may be different. For example, a mixture present in the cosmetic preparations according to the invention can comprise polyurethanes whose sections T are both branched, and/or those whose sections T are both linear, and/or those polyurethanes which comprise a linear section T and a branched section T. Such mixtures can of course also comprise other substances, such as e.g. further, preferably water-dispersible polyurethanes.

Such a mixing of polyurethanes PU can take place through the use corresponding to different feed materials or mixtures thereof in the preparation of the polyurethanes PU used in the preparations according to the invention, or be generated by subsequent mixing of only uniformly prepared polyurethanes used in the preparations according to the invention.

In one embodiment, the sum of the molecular weights of all sections T, plus the molecular weights of sections D is to be kept less than or equal to the sum of the molecular weights of all of the sections P.

The polyurethanes PU used in the preparations according to the invention can be prepared in the absence or preferably in the presence of at least one catalyst.

Suitable catalysts are, for example, all catalysts customarily used in polyurethane chemistry.

Particular preference is given to using those catalysts which are soluble in organic solvents such as xylene, toluene, acetone, tetrahydrofuran (THF), butyl acetate, N-methylpyrrolidone and/or N-ethylpyrrolidone.

Catalysts usually used in polyurethane chemistry are organic amines, in particular tertiary aliphatic, cycloaliphatic or aromatic amines, and Lewis-acidic organic metal compounds.

Suitable Lewis-acidic organic metal compounds are e.g. metal complexes such as acetyl acetonates of iron, titanium, zinc, aluminum, cobalt, manganese, nickel and zirconium, such as e.g. zirconium 2,2,6,6-tetramethyl-3,5-heptanedionate. Further suitable metal compounds are described by Blank et al. in Progress in Organic coatings, 1999, 35, 19 ff.

Bismuth, cobalt or zinc catalysts, and also cesium salts or titanium salts can also be used as catalysts.

Preferably, the preparation of the polyurethanes PU used in the preparations according to the invention takes place in the presence of compounds containing zinc and/or titanium. Particular preference is given to the presence of at least one zinc carboxylate or at least one titanium(IV) alcoholate or mixtures thereof in the preparation of the polyurethanes PU used in the preparations according to the invention.

For example, titanium alcoholates, preferably with a chain length of 2 or more carbon atoms, are used. In a preferred embodiment, the titanium alcoholates have a carbon chain of 20 or fewer carbon atoms. Preferably, the chain length of the titanium alcoholates is in the range from 3 to 18 carbon atoms. Particular preference is given to titanium alcoholates based on aliphatic alcohols. In a particularly preferred embodiment, the preparation of the polyurethanes PU used in the preparations according to the invention takes place in the presence of tetrabutyl orthotitanate, also known as titanium(IV) butylate or tetrabutoxytitanium.

In a preferred embodiment, the catalysts used are zinc carboxylates which are soluble in acetone, toluene, xylene and/or aliphatic hydrocarbons.

In a further preferred embodiment, the preparation of the polyurethanes PU used in the preparations according to the invention takes place in the presence of at least one zinc carboxylate in which the anion conforms to the formulae (CₙH₂ₙ₋₁O₂)— or (Cₙ₊₁H₂ₙ₋₂O₄)²⁻ where n is 1 to 20. Particularly preferred zinc salts have, as anions, monocarboxylates of the general formula (CₙH₂ₙ₋₁O₂)—, where n is the numbers 1 to 20.

Preferably, the polyurethanes PU used in the preparations according to the invention are prepared in the presence of zinc carboxylates, which are aliphatic or aromatic carboxylates, and if desired can comprise one or two ring structures.

In a particularly preferred embodiment, the catalysts for the preparation of the polyurethanes PU used in the preparations according to the invention are preferably zinc carboxylates whose carboxylic acid radicals have a carbon chain of 20 or fewer, preferably 18, particularly preferably less than or equal to 12 or fewer carbon atoms, since it has been found that in the case of long-chain carboxylate radicals, the activity of the catalyst in the process according to the invention decreases.

In one embodiment, zinc carboxylates without ring structure can be used as catalysts for preparing the polyurethanes used in the preparations according to the invention. Particular preference is given to using aliphatic zinc carboxylates as catalysts.

As catalysts, very particular preference is given to using zinc 2-ethylhexanoate (also called zinc octanoate), zinc n-octanoate, zinc n-decanoate, zinc neodecanoate, zinc ricinoleate and zinc stearate. Particular preference is given to using zinc neodecanoate.

It is of course also possible to use mixtures of two or more of the aforementioned compounds as catalysts for preparing the polyurethanes PU used in the preparations according to the invention. Preference is given to using only one catalyst.

The amount of catalyst used does not play a role per se. In general, a cost-effective amount of catalyst is used. Consequently, the catalyst or the mixture of catalysts is preferably used in an amount in the range from 100 ppm to 10 000 ppm, based on polyetherdiols used on a weight-basis. The catalyst is preferably used in an amount in the range from 500 to 5000 ppm, particularly preferably in an amount equal to or less than 4500 ppm, based on the weight of the total amount of all of the polyetherdiols used. In one particularly preferred embodiment, the catalyst is used in an amount in the range from 1000 ppm to 3000 ppm, based on the weight of the total amount of all of the polyetherdiols used.

The catalyst or catalysts can be added in solid or liquid form or in dissolved form, depending on the nature of the catalyst or the catalysts. Suitable solvents are water-immiscible solvents, such as aromatic or aliphatic hydrocarbons, inter alia toluene, xylene, ethyl acetate, hexane and cyclohexane, and also carboxylic acid esters, such as, for example, ethyl acetate. Furthermore, suitable solvents are acetone, THF and N-methylpyrrolidone and N-ethylpyrrolidone. Preferably, the catalyst or catalysts are added in solid or liquid form. Preferably, the catalyst is used in dissolved form in a solvent, very particularly preferably dissolved in organic solvents such as aliphatic hydrocarbons, acetone, toluene or xylene.

In a particularly preferred embodiment, the catalyst or catalysts are used in dissolved form.

In a further particularly preferred embodiment, the catalyst used is zinc carboxylates which are dissolved in aliphatic hydrocarbons, acetone, toluene, xylene or optionally mixtures thereof.

The polyurethanes PU used in the preparations according to the invention are prepared by a process according to the invention in which the synthesis takes place in two stages. If desired, the second reaction stage is followed by a work-up of the products.

In principle, the reaction can also be carried out without catalyst, although the products are generally more difficult to reproduce (with regard e.g. to the number-average and weight-average molecular weights), the reaction times are generally significantly longer and the viscosities achieved in preparations which comprise water are sometimes lower. In some cases, the increased formation of (high molecular weight) by-products resulted in crosslinking when no catalyst was present. Preferably, therefore, at least one, particularly preferably precisely one, catalyst is used.

One advantage of the process for preparing polyurethanes PU used in the preparations according to the invention in this preferred embodiment is the fact that the product comprises uniformly structured molecules or a clearly defined mixture of polyurethane molecules.

In one embodiment, the process for preparing polyurethanes PU used in the preparations according to the invention can comprise the following steps:
1. at least one polyetherdiol with a molecular weight of at least 1500 g/mol is reacted with at least one aliphatic diisocyanate and in the presence of at least one zinc carboxylate and/or at least one titanium alcoholate;
2. then the intermediates produced are reacted with at least one ethoxylated fatty alcohol;
3. then the work-up takes place, i.e. generally the removal of all organic solvents and the transfer of the polymer to water.

The reaction of the feed materials can take place in solution. A reaction in the melt is also possible, in which case the feed materials are present not in dissolved form or for the greatest part not in dissolved form in solvents.

In one preferred embodiment, the reaction is carried out in two steps in solution, particularly preferably dissolved in organic solvents such as acetone, toluene or xylene.

Preferably, polyetherdiol which is as anhydrous as possible is used in the first step. The removal of the water from the polyether can take place by azeotropic distillation, drying in vacuo or other methods known to the person skilled in the art. For example, through azeotropic distillation it is possible to remove water until the water content prior to the addition of the diisocyanates is approximately 300 ppm. The preparation of the actual reaction can, for example, consist of
either placing the polyetherdiol under reduced pressure and thus removing the water sufficiently (preferably to a water content of approximately 300 ppm or less), and then admixing a solvent, or
mixing the polyetherdiol with a solvent such as xylene, toluene or acetone and removing the water by azeotropic distillation, for example to a water content of approximately 300 ppm, where, however, the solvent is not completely removed, but the solution of polyether in the remaining solvent is used for the reaction in solution.

Prior to the reaction with diisocyanates, the pH of the diol solution in solvent can be adjusted to a value of less than or equal to pH 7 and, if desired, be buffered, for example by desalting or addition of an acid or mixture of different acids. Suitable acids are inorganic or organic acids, e.g. hydrochloric acid, sulfuric acid, sulfurous acid, nitric acid, phosphoric acid, hydrofluoric acid, carbonic acid, organic acids, such as malic acid, citric acid, oxalic acid, formic acid, acetic acid, propionic acid, butyric acid.

The ethoxylated fatty alcohols used preferably have a degree of ethoxylation which is at least in the range from 2 to 30 radicals, particularly preferably in the range from 3 to 25 ethylene oxide radicals and very particularly preferably in the range from 3 to 20 radicals. At least one of the fatty alcohols used is in most cases preferably a branched, nonionic compound prepared from a saturated iso-C13 alcohol of the structural formula RO(CH₂CH₂O)ₓH, where R is a C13-alkyl radical, preferably an iso-C13-alkyl radical, and where x=3, 5, 6, 6.5, 7, 8, 10, 12, 15 or 20, preferably x=10 (commercially available from BASF SE under the name “Lutensol®TO” e.g. when x=10 as “Lutensol®TO10”).

The ratio (mol to mol) of the polyetherdiols used to diisocyanates used can be in the range from 1:1.1 to 1:1.9. Preferably, the ratio is in the range from 1:1.1 to 1:1.8. The ratio is particularly preferably in the range from 1:1.1 to 1:1.75. The ratio is especially preferably in the range from 1:1.2 to 1:1.75. The ratio can of course also be 1:x where x is greater than or equal to 1.3, preferably x is greater than or equal to 1.5.

In one embodiment, this results in only one or two sections P preferably being present in one molecule of the polyurethanes used in the preparations according to the invention.

In a specific embodiment of the preparation process, in addition to the said ranges of the ratio of polyetherdiols to diisocyanates, the ratio of polyetherdiols to ethoxylated fatty alcohols is chosen so that the ratio (mol to mol) of polyetherdiols used to ethoxylated fatty alcohols used is in the range from 5:1 to 1:2. Preferably, this ratio (mol to mol) is in the range from 2:1 to 1:1.8, particularly preferably in the range from 1:1 to 1:1.6 and most preferably 1:1.5.

For all three feed materials, it is the case that a ratio (mol to mol) of polyetherdiols to diisocyanates to ethoxylated fatty alcohols of 1:1.75:1.5 is very particularly preferably used.

Preferred cosmetic preparations according to the invention which comprise the polyurethanes PU described above are those which also comprise water. In this connection, preference is given to cosmetic preparations which comprise at least 5% by weight, in particular at least 20% by weight, very particularly preferably at least 30% by weight and most preferably at least 50% by weight, of water. The cosmetic preparations comprising water may be, for example, solutions, emulsions, suspensions or dispersions.

A preferred embodiment of the invention is preparations which, besides the polyurethanes PU, comprise water and at least one salt or at least one surfactant or mixtures thereof.

Within the context of the present invention, surfactants are also understood as meaning emulsifiers and mixtures of surfactants and emulsifiers. Within the context of the present invention, salt is understood as meaning salts and also salt-like structures also with a low pK_{S} value and mixtures thereof.

Particular preference is given to preparations according to the invention which, besides the polyurethanes PU, comprise at least 0.05% by weight of salt and/or at least 0.5% by weight of surfactants, very particularly preferably at least 0.1% by weight of salt and/or at least 1% by weight of surfactants.

A further embodiment is preparations which, besides the polyurethanes PU, comprise up to 20% by weight of salt, preferably up to 10% by weight and particularly preferably up to 5% by weight of salt.

A further embodiment is preparations comprising polyurethane PU and up to 25% by weight of surfactants, preferably up to 20% by weight and particularly preferably 15% by weight of surfactants.

A further embodiment is preparations comprising polyurethane PU and up to 10% by weight of salt, preferably up to 5% by weight of salt and up to 20% by weight of surfactants, preferably up to 15% by weight of surfactants.

A further embodiment is salt-free or salt-reduced surfactant systems.

A particularly preferred embodiment is preparations comprising polyurethane PU in the form of oil-in-water emulsions (O/W emulsions). Typically, oil-in-water emulsions comprise an oil fraction greater than 0% by weight and less than or equal to 40% by weight. Preference is given to oil-in-water emulsions which comprise an oil fraction in the range from 5 to 40% by weight, particularly in the range from 10 to 35% by weight and in particular from 15 to 30% by weight of oil.

Very particular preference is given to preparations comprising polyurethanes PU which are oil-in-water emulsions and comprise at least one salt.

The cosmetic preparations according to the invention comprise the polyurethanes PU preferably in an amount of from 0.01 to 10% by weight, preferably 0.05 to 5% by weight, particularly preferably 0.1 to 1.5% by weight, based on the weight of the preparation.

To produce the preparations according to the invention, which may be for example solutions, emulsions, suspensions or dispersions, the polyurethanes PU are preferably used in the form of aqueous dispersions, as can be obtained from the preparation process by work-up (for example by removing the solvent, adding water and, if desired, by adding a preservative and/or a stabilizer).

In the cosmetic or dermatological preparations, preference is given to using polyurethanes PU whose 10 percent strength by weight aqueous dispersions have a dynamic viscosity, measured as described below at a shear rate of 100 1/s, of at least 100 mPa*s, particularly preferably of at least 200 mPa*s and very particularly preferably of at least 300 mPa*s. The aqueous dispersions of the polyurethanes PU here can exhibit either Newtonian behavior or structurally viscous behavior. Structurally viscous dispersions which comprise the polyurethanes PU preferably have dynamic viscosities of at least 1000 mPa*s, particularly preferably even of at least 3000 mPa*s (10% strength by weight aqueous dispersions, measured as described below at a shear rate of 100 1/s).

The person skilled in the art is aware that in preparations comprising water, many thickeners forfeit their effect, i.e. the viscosity of the preparation drops as soon as the preparations further comprise salt and/or surfactant. By contrast, in a preferred embodiment, the polyurethanes PU lead to a stabilization of the viscosity of preparations comprising water even with added salt and/or surfactant.

In a further embodiment, the viscosity of preparations comprising water which comprise at least one salt is kept approximately constant or is even increased through the presence of the polyurethanes PU in the preparation compared to preparations which comprise only salt or only polyurethanes PU. Here, the order in which polyurethanes PU and salt are added is unimportant.

Particular preference is given to polyurethanes PU which lead to a high tolerance or even increase in the dynamic viscosity, measured as described below, of preparations comprising water if at least one salt or at least one surfactant or preferably mixtures thereof are present in the preparations.

Particular preference is given to the use of polyurethanes PU which, at a salt concentration of greater than or equal to 0.5% by weight, following addition, lead to a stabilization of the dynamic viscosity, measured as described below, of preparations comprising water. Particular preference is given to the use of polyurethanes PU which lead to a stabilization of the dynamic viscosity upon adding greater than or equal to 0.5% by weight of salt and adding greater than or equal to 1% by weight of surfactant, the order of the additions, if desired, being unimportant.

Particular preference is given to the use of polyurethanes PU which lead to an increase in the dynamic viscosity, measured as described below, of preparations comprising water if at least one salt or at least one surfactant or mixtures thereof are present in the preparations. Particular preference is given to polyurethanes PU which, at a salt concentration of greater than or equal to 0.5% by weight, lead to an increase in the dynamic viscosity, measured as described below, of preparations comprising water. Particular preference is given to those polyurethanes which lead to an increase in the dynamic viscosity compared to preparations which comprise less than 0.5% by weight, preferably 0.1% by weight, of salt, or less than 1% by weight, preferably 0.5% by weight, of surfactant.

Very particular preference is given to polyurethanes PU which, at a salt concentration of greater than or equal to 0.05% by weight, lead to an increase in the dynamic viscosity, measured as described below, of preparations comprising water. Particular preference is given to those polyurethanes which lead to an increase in the dynamic viscosity compared to preparations which comprise less than 0.05% by weight, preferably less than or equal to 0.01% by weight, of salt, or less than 0.5% by weight, preferably less than or equal to 0.1% by weight of surfactant.

One embodiment of the invention is cosmetic preparations according to the invention which comprise at least 0.5% by weight of at least one salt and at least 1% by weight of at least one surfactant.

The mode of action of the polyurethanes PU described above is largely independent of the charge density or ionic strength of the other ingredients of the preparations according to the invention. The effectiveness of the effect of the polyurethanes PU is comparable for mono- or polyvalent ions.

### pH Range

In contrast to many other rheology modifiers, the polyurethanes PU described above can be used in a large pH range from pH=2 to pH=12.

A further advantage of the polyurethanes is the micelle formation in water. The critical micelle concentration (CMC) indicates the smallest possible concentration of a substance, mostly of a substance which has hydrophobic and hydrophilic sections, at which micelles are spontaneously formed. The CMC of the polyurethanes PU in water, determined as described below, is preferably less than or equal to 1 g/l, particularly preferably less than or equal to 0.5 g/l, especially preferably less than or equal to 0.25 g/l and very particularly preferably less than or equal to 0.1 g/l.

A further advantage of the preparations according to the invention is the preferred use of zinc- and/or titanium-containing catalysts in the preparation of the polyurethanes PU. Particularly in the field of cosmetic preparations, the processes known from the prior art using tin are no longer desired since tin may also be present in the products and preparations resulting therefrom. Zinc-containing additives of cosmetic preparations are accepted, where zinc can confer additional advantages through its antibacterial and antiinflammatory properties.

The cosmetic or dermatological preparations according to the invention comprise at least one polyurethane PU and at least one cosmetically acceptable carrier.

The cosmetically acceptable carrier is preferably selected from

i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trivalent alcohols different from iii),
v) saturated acyclic and cyclic hydrocarbons,
vi) fatty acids,
vii) fatty alcohols,
viii) propellant gases,
ix) mixtures thereof.

Thus, for example, hydrophilic carriers such as water or mono-, di- or polyhydric alcohols having preferably 1 to 8 carbon atoms, such as ethanol, n-propanol, iso-propanol, propylene glycol, glycerol and sorbitol, are suitable cosmetically acceptable carriers.

### Diols

Diols are particularly suitable as hydrophilic carriers. Diols can advantageously be incorporated into cosmetic preparations in combination with polyurethane PU, as a result of which an increase in effectiveness of polyurethane PU can be observed. In particular, the use of propylene glycol in the presence of PU is advantageous. Advantageous use concentrations of the diols, preferably of propylene glycol, are in the range from 1 to 10% by weight, based on the total weight of the preparation.

The invention thus further provides cosmetic preparations according to the invention comprising polyurethane PU and in the region of from 1 to 10% by weight of at least one diol.

The preparations according to the invention can be formulated as aqueous or aqueous-alcoholic solutions, O/W (preferably) and W/O emulsions, hydrodispersion formulations, solids-stabilized formulations, stick formulations, PIT formulations, in the form of creams, foams, sprays (pumpspray or aerosol), gels, gelsprays, lotions, oils, oil gels or mousse, and accordingly be formulated with customary further auxiliaries.

The cosmetic preparations according to the invention may be skin cosmetic, hair cosmetic, dermatological, hygiene or pharmaceutical preparations. Preferably, the preparations according to the invention are present in the form of a gel, foam, spray, ointment, cream, emulsion, suspension, lotion, milk or paste. If desired, liposomes or microspheres can also be used.

In particular, different administration forms of a sprayable or low-viscosity formulation can be achieved by adding different amounts of PU during or after the preparation of the sprayable or low-viscosity formulation.

In one embodiment of the invention, a low-viscosity cosmetic composition which already comprises all desired other constituents is prepared. After all desired other constituents have been added, the required amount of polyurethane PU is added to establish the desired viscosity. Thus, for example, some of a low-viscosity formulation can be formulated without the addition of polyurethane PU in the form of a spray, and some can be formulated as lotion by adding polyurethane PU, and finally, by adding more polyurethane PU, be formulated as a cream. In the event of the subsequent addition of polyurethane PU, the viscosity does not necessarily have to be adjusted by means of a post-homogenization; in contrast to established thickener systems, simple stirring suffices to achieve the desired effect. The option of establishing the desired viscosity of cosmetic preparations subsequently, i.e. after all of the constituents with the exception of the thickener are already present, by adding the thickener is one of the advantage of the polyurethanes PU.

The polyurethane PU can therefore be added while or after preparing the preparation which comprises all otherwise desired constituents.

The cosmetic preparations according to the invention in the form of oil-in-water or water-in-oil emulsions can be prepared by mixing the corresponding oil and water phases, in which one of the two phases is hot and the other is cold, the two phases are cold or the two phases are hot. “Hot” here means a temperature of from about 70° C. to 80° C., “cold” means a temperature of from about 20° C. to 30° C.

The polyurethane PU may be present in the oil phase and/or the water phase, it preferably being present in the water phase.

The invention relates preferably to cosmetic preparations which are selected from gels, gel creams, milks, hydroformulations, stick formulations, cosmetic oils and oil gels, mascara, self-tanning compositions, facecare compositions, bodycare compositions, aftersun preparations. The term cosmetic preparations is also understood as meaning preparations for oral care.

Further cosmetic preparations according to the invention are skin cosmetic preparations, in particular those for skincare. These are in particular in the form of W/O or preferably O/W skin creams, day creams and night creams, eye creams, face creams, antiwrinkle creams, mimic creams, moisturizing creams, bleaching creams, vitamin creams, skin lotions, care lotions and moisturizing lotions.

Further preferred preparations according to the invention are face masks, cosmetic lotions and preparations for use in decorative cosmetics, for example for concealing sticks, stage make-up, mascara and eyeshadows, lipsticks, kohl pencils, eyeliners, make-ups, foundations, blushers, powders and eyebrow pencils.

Further preparations according to the invention are antiacne compositions, repellants, shaving compositions, hair removal compositions, intimate care compositions, footcare compositions, and babycare products.

Further preferred preparations according to the invention are washing, showering and bathing preparations. Within the context of this invention, washing, showering and bathing preparations are soaps from liquid to gel-like consistency, transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and shower gels, foam baths, oil baths and scrub preparations, shaving foams, lotions and creams.

Suitable further ingredients for the aforementioned preparations according to the invention are described below.

Besides the polyurethanes PU and the carrier as defined above, preferred preparations according to the invention comprise one or more further cosmetically acceptable additives such as, for example, emulsifiers and coemulsifiers, solvents, surfactants, oil bodies, preservatives, perfume oils, cosmetic care substances and active ingredients such as AHAs, fruit acids, ceramides, phytantriol, collagen, vitamins and provitamins, for example vitamin A, E and C, retinol, bisabolol, panthenol, natural and synthetic photoprotective agents, natural substances, opacifiers, solubility promoters, repellents, bleaches, colorants, tinting agents, tanning agents (e.g. dihydroxyacetone), micropigments such as titanium oxide or zinc oxide, superfatting agents, pearlescent waxes, solubilizers, complexing agents, fats, waxes, silicone compounds, hydrotropes, dyes, stabilizers, pH regulators, reflectors, proteins and protein hydrolysates (e.g. wheat, almond or pea proteins), ceramide, protein hydrolysates, salts, gel formers, further consistency regulators, further thickeners, silicones, humectants, (e.g. 1,2-pentanediol), refatting agents, UV photoprotective filters, film-forming polymers, conditioning polymers, antioxidants, antifoams, antistats, emollients, softeners, peroxides and further customary additives.

### Antioxidants

A content of antioxidants in the preparations according to the invention is generally preferred. According to the invention, antioxidants which can be used are all antioxidants suitable or customary for cosmetic applications. Suitable antioxidants for the preparations according to the invention are described for example on page 41, line 12 to page 42, line 33 of WO 2006/106140. Reference is hereby made to the contents of the cited reference in their entirety.

### Oils, Fats and Waxes

In a preferred embodiment, besides the polyurethanes PU and the cosmetically acceptable carrier, the preparations according to the invention have an oil component, fat component and/or wax component which is selected from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; vaseline; esters, preferably esters of fatty acids, such as e.g. the esters of C₁-C₂₄-monoalcohols with C₁-C₂₂-monocarboxylic acids, such as isopropyl isostearate, n-propyl myristate, iso-propyl myristate, n-propyl palmitate, isopropyl palmitate, hexacosanyl palmitate, octacosanyl palmitate, triacontanyl palmitate, dotriacontanyl palmitate, tetratriacontanyl palmitate, hexanecosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkylbenzoates, benzyl benzoate; other cosmetic esters, such as fatty acid triglycerides, propylene glycol monolaurate, polyethylene glycol monolaurate, C₁₀-C₁₅-alkyl lactates, and mixtures thereof.

The oil or wax component can also be selected from silicone oils and derivatives thereof, such as, e.g. linear polydimethylsiloxanes, poly(methylphenyl)siloxanes, cyclic siloxanes and mixtures thereof. The number-average molecular weight of the polydimethylsiloxanes and poly(methylphenyl)siloxanes is preferably in a range from about 1000 to 150 000 g/mol. Preferred cyclic siloxanes have 4- to 8-membered rings. Suitable cyclic siloxanes are commercially available e.g. under the name cyclomethicone.

Preferred oil and fat components are selected from paraffin and paraffin oils; vaseline; natural fats and oils, such as castor oil, soybean oil, peanut oil, olive oil, sunflower oil, sesame oil, avocado oil, cocoa butter, almond oil, peach kernel oil, ricinus oil, cod liver oil, pig grease, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti, and mixtures of the aforementioned oil and fat components.

Suitable cosmetically and pharmaceutically compatible oil and fat components are also described in Karl-Heinz Schrader, Grundlagen and Rezepturen der Kosmetika [Fundamentals and formulations of cosmetics], 2nd edition, Verlag Hüthig, Heidelberg, pp. 319-355, to which reference is hereby made.

Advantageously, those oils, fats and/or waxes are selected which are described on page 28, line 39 to page 34, line 22 of WO 2006/106140. Reference is hereby made to the contents of the cited reference in their entirety.

The content of oils, fats and waxes is at most 80, preferably 50, further preferably at most 30% by weight, based on the total weight of the preparation according to the invention.

### Surfactants

Besides the polyurethanes PU and the at least one cosmetically acceptable carrier, preferably water, preferred cosmetic preparations furthermore comprise at least one surfactant. Surfactants which can be used are anionic, cationic, nonionic and/or amphoteric surfactants.

Advantageous washing-active anionic surfactants within the context of the present invention are acylamino acids and salts thereof, such as acyl glutamates, in particular sodium acyl glutamate
sarcosinates, for example myristoyl sarcosine, TEA lauroyl sarcosinate, sodium lauroyl sarcosinate and sodium cocoyl sarcosinate, sulfonic acids and salts thereof, such as
acyl isethionates, for example sodium or ammonium cocoyl isethionate
sulfosuccinates, for example dioctyl sodium sulfosuccinate, disodium laureth sulfosuccinate, disodium lauryl sulfosuccinate and disodium undecylenamido MEA sulfosuccinate, disodium PEG-5 lauryl citrate sulfosuccinate and derivatives,
alkyl ether sulfates, for example sodium, ammonium, magnesium, MIPA, TIPA laureth sulfate, sodium myreth sulfate and sodium C₁₂₋₁₃ pareth sulfate,
alkyl ether sulfonates, for example sodium C12-15 pareth-15 sulfonate
alkyl sulfates, for example sodium, ammonium and TEA lauryl sulfate.

Further advantageous anionic surfactants are
taurates, for example sodium lauroyl taurate and sodium methylcocoyl taurate,
ether carboxylic acids, for example sodium laureth-13 carboxylate and sodium PEG-6 cocamide carboxylate, sodium PEG-7 olive oil carboxylate
phosphoric acid esters and salts, such as for example DEA-oleth-10 phosphate and dilaureth-4 phosphate,
alkylsulfonates, for example sodium coconut monoglyceride sulfate, sodium C₁₂₋₁₄ olefinsulfonate, sodium lauryl sulfoacetate and magnesium PEG-3 cocamidosulfate,
acyl glutamates such as di-TEA palmitoyl aspartate and sodium caprylic/capric glutamate,
acylpeptides, for example palmitoyl hydrolyzed milk protein, sodium cocoyl hydrolyzed soybean protein and sodium/potassium cocoyl hydrolyzed collagen and also carboxylic acids and derivatives, such as, for example, lauric acid, aluminum stearate, magnesium alkanolate and zinc undecylenate, ester carboxylic acids, for example calcium stearoyl lactylate, laureth-6 citrate and sodium PEG-4 lauramide carboxylate
alkylarylsulfonates.

A preferred embodiment of the invention is cosmetic preparations which comprise at least one polyurethane PU as defined above and at least one hydrophobically modified phosphate and/or at least one hydrophobically modified sulfate. By way of example, hydrophobically modified phosphates which may be mentioned are Luviquat®Mono CP (INCI: Hydroxyethyl Cetyldimonium Phosphate), Luviquat®Mono LS (INCI: Cocotrimonium Methosulfate), Amphisol® grades (INCI: Cetyl Phosphate, Potassium Cetyl Phosphate) and a hydrophobically modified sulfate that may be mentioned is Lanette®E (INCI: Sodium Cetearyl Sulfate). Hydrophobically modified phosphates and/or sulfates of this type lead, in combination with the polyurethanes PU, to a further increase in viscosity. This increase in viscosity is also retained when increasing the temperature of the preparation.

It is preferred if the weight ratio of polyurethane PU to hydrophobically modified phosphate and/or hydrophobically modified sulfate is in the range from 0.5:1 to 2:1, preferably in the range from 0.8:1 to 1.2:1.

Advantageous washing-active cationic surfactants within the context of the present invention are quaternary surfactants. Quaternary surfactants comprise at least one N atom which is covalently bonded to 4 alkyl or aryl groups. For example, alkylbetaine, alkylamidopropylbetaine and alkylamidopropylhydroxysultaine are advantageous. Further advantageous cationic surfactants within the context of the present invention are also
alkylamines,
alkylimidazoles and
ethoxylated amines
   and in particular salts thereof.

Advantageous washing-active amphoteric surfactants within the context of the present invention are acyl-/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut-fatty acid amidoethyl-N-hydroxyethyl glycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous washing-active nonionic surfactants within the context of the present invention are
alkanolamides, such as cocamides MEA/DEA/MIPA,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers, alkyl polyglycosides, such as lauryl glucoside, decyl glycoside and cocoglycoside, glycosides with a HLB value of at least 20 (e.g. Belsil®SPG 128V (Wacker)).

Further advantageous nonionic surfactants are alcohols and amine oxides, such as cocoamidopropylamine oxide.

Preferred anionic, amphoteric and nonionic surfactants are specified for example in “Kosmetik and Hygiene von Kopf bis Fuβ [Cosmetics and hygiene from head to toe]”, ed. W. Umbach, 3rd edition, Wiley-VCH, 2004, pp. 131-134. to which reference is made at this point in its entirety.

Among the alkyl ether sulfates, preference is given in particular to sodium alkyl ether sulfates based on di- or triethoxylated lauryl and myristyl alcohol. They are considerably superior to the alkyl sulfates in terms of the insensitivity towards water hardness, ability to be thickened, solubility at low temperatures and in particular skin and mucosa compatibility. They can also be used as sole washing raw materials for shampoos. Lauryl ether sulfate has better foaming properties than myristyl ether sulfate, but is inferior to it in terms of mildness.

Alkyl ether carboxylates with an average and particularly with a high belong to the mildest surfactants generally, but exhibit a poor foaming and viscosity behavior. They are often used in combination with alkyl ether sulfates and amphoteric surfactants in hair washing compositions.

Sulfosuccinic acid esters (sulfosuccinates) are mild and readily foaming surfactants but, on account of their poor ability to be thickened, are preferably used only together with other anionic and amphoteric surfactants and, on account of their low hydrolysis stability, are used preferably only in neutral or well buffered products.

Amidopropylbetaines are practically insignificant as sole washing raw materials since their foaming behavior and also their ability to be thickened are only moderate. By contrast, these surfactants have exceptional skin and eye mucosa compatibility. In combination with anionic surfactants, their mildness can be synergistically improved. Preference is given to the use of cocamidopropylbetaine.

Amphoacetates/amphodiacetates, being amphoteric surfactants, have very good skin and mucosa compatibility and can have a hair-conditioning effect and/or increase the care effect of additives. Similarly to the betaines, they are used for optimizing alkyl ether sulfate formulations. Sodium cocoamphoacetate and disodium cocoamphodiacetate are most preferred.

Alkyl polyglycosides are nonionic washing raw materials. They are mild, have good universal properties, but are weakly foaming. For this reason, they are preferably used in combinations with anionic surfactants.

Sorbitan esters likewise belong to the nonionic washing raw materials. On account of their exceptional mildness, they are preferably used for use in baby shampoos. Being low-foamers, they are preferably used in combination with anionic surfactants. According to the invention, it is advantageous if one or more of these surfactants are used in a concentration of from 0.1 to 30% by weight, preferably in a concentration of from 1 to 25% by weight, further preferably in a concentration of from 5 to 25% by weight and very particularly preferably in a concentration of from 10 to 20% by weight, in each case based on the total weight of the preparation.

### Inclusion of Air

A further advantage of the cosmetic preparations comprising the polyurethanes PU is that up to 150% by volume, based on the volume of the preparation prior to the introduction of air, of air can be introduced. The invention thus further provides a cosmetic preparation according to the invention obtainable by introducing air into the preparation otherwise already comprising all other constituents in the range from 5 to 150% by volume, based on the volume of the preparation prior to introducing the air. Such preparations comprising from 5 to 150% by volume, preferably from 20 to 100% by volume, of air are further provided by the present invention. The preparations according to the invention comprising from 5 to 150% by volume, based on the volume of the preparation before the introduction of air, of air are volume-stable over several months. The introduction of air leads to an improvement in structure (narrow size distribution of the air bubbles), sensory properties and visual impression. Examples of such advantageous embodiments of the invention are mousse preparations.

### Polysorbates

Polysorbates can also advantageously be incorporated into the preparations according to the invention. Polysorbates advantageous within the context of the invention are
Polyoxyethylene(20) sorbitan monolaurate (Tween®20, CAS No. 9005-64-5)
Polyoxyethylene(4) sorbitan monolaurate (Tween®21, CAS No. 9005-64-5)
Polyoxyethylene(4) sorbitan monostearate (Tween®61, CAS No. 9005-67-8)
Polyoxyethylene(20) sorbitan tristearate (Tween®65, CAS No. 9005-71-4)
Polyoxyethylene(20) sorbitan monooleate (Tween®80, CAS No. 9005-65-6)
Polyoxyethylene(5) sorbitan monooleate (Tween®81, CAS No. 9005-65-5)
Polyoxyethylene(20) sorbitan trioleate (Tween®85, CAS No. 9005-70-3).

Those which are particularly advantageous are
Polyoxyethylene(20) sorbitan monopalmitate (Tween®40, CAS No. 9005-66-7) and
Polyoxyethylene(20) sorbitan monostearate (Tween®60, CAS No. 9005-67-8).

The polysorbates are advantageously used in a concentration of from 0.1 to 5 and in particular in a concentration of from 1.5 to 2.5% by weight, based on the total weight of the preparation, individually or as a mixture of two or more polysorbates.

### Conditioning Agents

Besides the polyurethanes PU and at least one cosmetically acceptable carrier, preferably water, preferred cosmetic preparations further comprise at least one conditioning agent. The conditioning agents selected for the cosmetic preparations according to the invention are preferably those conditioning agents which are described on page 34, line 24 to page 37, line 10 of WO 2006/106140. Reference is hereby made to the content of the cited passage in its entirety. Further preferred conditioning agents are hydrogenated polyisobutenes, in particular that commercially available as Luvitol®Lite.

### Rheology Modifying Agents

In general, the rheology of the preparations according to the invention can be adjusted to the value desired in each case through the polyurethanes PU. The use of further rheology modifying agents is not automatically necessary. Of course, however, further thickeners can additionally be used in the preparations according to the invention. Thickeners suitable for gels, shampoos and haircare compositions are given in “Kosmetik and Hygiene von Kopf bis Fuβ [Cosmetics and hygiene from head to toe]”, ed. W. Umbach, 3rd edition, Wiley-VCH, 2004, pp. 235-236, to which reference is made at this point in its entirety. Suitable further thickeners for the cosmetic preparations according to the invention are described for example also on page 37, line 12 to page 38, line 8 of WO 2006/106140. Reference is also made to the contents of this passage in its entirety.

In one embodiment of the invention, the preparations according to the invention do not comprise any further rheology modifying agents apart from the polyurethanes PU. The present invention further provides cosmetic preparations which, apart from the polyurethanes PU, comprise further rheology modifying agents. One embodiment of the invention is cosmetic preparations which comprise polyurethanes PU and polyacrylate thickeners, in particular hydrophobically modified polyacrylate thickeners. A preferred embodiment of the invention is cosmetic preparations comprising at least one polyurethane PU and at least one polyacrylate thickener with the INCI name Acrylates/C10-30 Alkyl Acrylate Crosspolymer (e.g. Carbopol®Ultrez 21).

Cosmetic preparations which comprise at least one polyurethane PU and at least one Acrylates/C10-30 Alkyl Acrylate Crosspolymer are, for example, gels which have improved sensory properties for the same viscosity.

The combination of polyurethanes PU with hydrophobically modified polyacrylates leads to synergistic effects as regards thickening. Combinations of polyurethanes PU with polyacrylates, in particular hydrophobically modified polyacrylates, lead to an identical or greater thickening effect compared to the sole use of polyacrylate thickeners for a simultaneously reduced use amount. The resulting formulations have light sensory properties and exceptional spreadability on for example the skin.

The invention further provides a method of improving the spreadability of cosmetic preparations, in particular in the form of emulsions, on the skin, wherein at least one polyurethane PU is added to the cosmetic preparation. such that the quantitative fraction of polyurethane PU, based on the total weight of the preparation, is in the range from 0.1 to 10% by weight.

### Preservatives

Preparations with high water contents in particular have to be reliably protected against the build-up of germs. In one preferred embodiment, the cosmetic preparations according to the invention comprise preservatives.

Suitable preservatives for the cosmetic compositions according to the invention are described for example on page 38, line 10 to page 39, line 18 of WO 2006/106140. Reference is hereby made to the content of the cited passage in its entirety.

### Complexing Agents

Since the raw materials and also many cosmetic compositions themselves are manufactured predominantly in steel apparatuses, the end products can comprise iron (ions) in trace amounts. In order to prevent these impurities adversely affecting the product quality via reactions with dyes and perfume oil constituents, in one preferred embodiment, complexing agents such as salts of ethylenediamine-tetraacetic acid, of nitrilotriacetic acid, of iminodisuccinic acid or phosphates are added to the cosmetic preparations.

### UV Photoprotective Filters

In order to stabilize the UV-light-sensitive ingredients present in the cosmetic preparations according to the invention, such as, for example, dyes and perfume oils, against changes due to UV light, UV photoprotective filters, such as e.g. benzophenone derivatives, can be incorporated. Suitable UV photoprotective filters for the cosmetic compositions according to the invention are described for example on page 39, line 20 to page 41, line 10 of WO 2006/106140. Reference is hereby made to the content of the cited passage in its entirety. Further suitable UV photoprotective filters are specified below in connection with cosmetic UV photoprotective preparations.

### Buffers

In a preferred embodiment of the invention, the cosmetic preparations comprise buffers. Buffers ensure the pH stability of the cosmetic preparations. Citrate, lactate and phosphate buffers are predominantly used.

### Solubility Promoters

In one preferred embodiment of the invention, the cosmetic preparations comprise solubility promoters. Solubility promoters are used in order to convert care oils or perfume oils to a clear solution and to keep them in clear solution even at low temperature. The most common solubility promoters are ethoxylated nonionic surfactants, e.g. hydrogenated and ethoxylated castor oils. The polyurethanes PU used according to the invention can themselves act as solubility promoters.

### Antimicrobial Agents

In one preferred embodiment of the invention, the cosmetic preparations comprise antimicrobial agents. The antimicrobial agents generally include all suitable preservatives with a specific effect against Gram-positive bacteria, e.g. triclosan (2,4,4′-trichloro-2′-hydroxydiphenyl ether), chlorhexidine (1,1′-hexamethylenebis[5-(4-chlorophenyl)biguanide), and TTC (3,4,4′-trichlorocarbanilide). Quaternary ammonium compounds are in principle likewise suitable and are preferably used for disinfecting soaps and washing lotions. Numerous fragrances also have antimicrobial properties. A large number of essential oils and/or their characteristic ingredients, such as e.g. clove oil (eugenol), mint oil (menthol) or thyme oil (thymol), also exhibit marked antimicrobial effectiveness. The antibacterially effective substances are used for the preparations generally in concentrations of ca. 0.1 to 0.3% by weight, based on the preparation.

### Dispersants

If sparingly soluble or insoluble active ingredients, e.g. antidandruff active ingredients or silicone oils, are to be dispersed or held permanently in suspension in the preparations according to the invention, it is necessary to use dispersants and thickeners such as e.g. magnesium aluminum silicates, bentonites, fatty acyl derivatives, polyvinylpyrrolidone or hydrocolloids, e.g. xanthan gum or carbomers. According to the invention, dispersants are present in a total concentration of at most 2, preferably at most 1.5 and particularly preferably at most 1% by weight, based on the total weight of the preparation.

In one preferred embodiment, the preparations according to the invention such as gels, shampoos and haircare compositions comprise ethoxylated oils selected from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride PEG-6 ester, PEG-20 corn oil glycerides, PEG-18 glyceryl oleate cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 “Evening Primrose” glycerides, PEG-200 hydrogenated glyceryl palmate, PEG-90 glyceryl isostearate.

Preferred ethoxylated oils are PEG-7 glyceryl cocoate, PEG-9 cocoglycerides, PEG-40 hydrogenated castor oil, PEG-200 hydrogenated glyceryl palmate.

Ethoxylated glycerol fatty acid esters are used in aqueous cleaning formulations for various purposes. Glycerol fatty acid esters with a degree of ethoxylation of ca. 30-50 serve as solubility promoters for nonpolar substances such as perfume oils. Highly ethoxylated glycerol fatty acid esters are used as thickeners.

### Active Ingredients

In a preferred embodiment, the preparations according to the invention comprise cosmetically and/or dermatologically active ingredients. Advantageous active ingredients for the cosmetic preparations according to the invention are described for example on page 44, line 24 to page 49, line 39 of WO 2006/106140. Reference is hereby made to the content of the cited passage in its entirety.

A further embodiment of the invention is cosmetic preparations which comprise at least one polyurethane PU and from 0.1 to 20% by weight, preferably from 0.5 to 15% by weight, further preferably from 5 to 12% by weight, of urea. Even large amounts of urea can be incorporated into the cosmetic preparations stably with the simultaneous presence of polyurethane PU and with establishment of a required viscosity.

### Pearlescent Waxes

In a preferred embodiment, the preparations according to the invention comprise pearlescent waxes. Suitable pearlescent waxes for the cosmetic preparations according to the invention are described for example on page 50, line 1 to line 16 of WO 2006/106140. Reference is hereby made to the content of the cited passage in its entirety.

The preparations according to the invention can furthermore comprise glitter substances and/or other effect substances (e.g. colored streaks).

### Emulsifiers

In a preferred embodiment of the invention, the cosmetic preparations according to the invention are present in the form of emulsions, preferably O/W emulsions. Such emulsions are prepared by known methods. Suitable emulsifiers for the emulsions according to the invention are described for example on page 50, line 18 to page 53, line 4 of WO 2006/106140. Reference is hereby made to the content of the cited passage in its entirety.

### Perfume Oils

In a preferred embodiment, the preparations according to the invention comprise perfume oils. Suitable perfume oils are described for example on page 53, line 10 to page 54, line 3 of WO 2006/106140. Reference is hereby made to the content of the cited passage in its entirety.

### Pigments

In a preferred embodiment, the preparations according to the invention further comprise pigments. The pigments are present in the product in most cases in undissolved form and may be present in an amount of from 0.05 to 90% by weight, particularly preferably from 1 to 15% by weight. In one embodiment of the invention, in particular in the case of preparations in the form of decorative cosmetics, such as, for example, eyeshadows, the pigment amount can be up to 90% by weight of the preparation.

The preferred particle size is 0.01 to 200 μm, in particular 0.02 to 150 μm, particularly preferably 0.05 to 100 μm.

Suitable pigments for the compositions according to the invention are described for example on page 54, line 5 to page 55, line 19 of WO 2006/106140. Reference is hereby made to the content of the specified passage in its entirety.

### Nanoparticles

In a preferred embodiment, the preparations according to the invention further comprise pigments in the form of water-insoluble nanoparticles, i.e. particles with a particle size in the range from 1 to 200, preferably from 5 to 100 nm. Preferred nanoparticles are nanoparticles of metal oxides, in particular of zinc oxide and/or titanium dioxide and/or silicon dioxide.

### Polymers

In a preferred embodiment, the cosmetic preparations according to the invention comprise further polymers apart from the polyurethanes PU. Preferred further polymers are water-soluble or water-dispersible polymers, particular preference being given to water-soluble polymers.

Further polymers suitable for the preparations according to the invention are described for example on page 55, line 21 to page 63, line 2 of WO 2006/106140. Reference is hereby made to the content of the specified passage in its entirety.

Cosmetic and/or Dermatological Photoprotective Compositions

Within the context of this invention, cosmetic and dermatological photoprotective compositions are understood as meaning cosmetic and dermatological preparations which comprise at least one, preferably two or more, UV filter substances.

The term UV filter substance is known to the person skilled in the art and refers to substances or preparations which are added to cosmetic or dermatological compositions for the purpose of filtering UV rays in order to protect the skin against certain harmful effects of this radiation. In the narrower sense, it also refers to compounds which absorb light with wavelengths in the UV region. Depending on the absorption spectrum, a distinction is made between UV-A, UV-B and UV-C filters. As a rule, UV filters are used as combinations for satisfying spectral and formulation-related requirements. Pigments and micropigments preferably of titanium dioxide and/or zinc oxide (see above), which are predominantly used for the aforementioned purpose, can also be referred to as UV filters.

Cosmetic preparations according to the invention can of course also be simultaneously suitable for cosmetic and dermatological purposes. The expression “cosmetic or dermatological photoprotective compositions” accordingly also encompasses those compositions which simultaneously fulfil both cosmetic and dermatological purposes.

A preferred embodiment of the present invention relates to photoprotective compositions which have an SPF of at least 4, including in particular those cosmetic or dermatological photoprotective compositions which comprise at least one UV filter substance.

The cosmetic and/or dermatological photoprotective preparations of this invention serve for cosmetic and/or dermatological photoprotection, and also for the treatment and care of the skin and/or the hair and as make-up product in decorative cosmetics. The cosmetic and/or dermatological photoprotective preparations of the present invention include for example suncreams, sun lotions, sun milks, sun oils, sun balsams, sun gels, lip care and lipsticks, concealing creams and concealing sticks, moisturizing creams, moisturizing lotions, moisturizing emulsions, face, body and hand creams, hair treatments and rinses, hair-setting compositions, styling gels, hair sprays, deodorant roll-ons or eye wrinkle creams, tropicals, sunblocks, aftersun preparations. All of these photoprotective preparations comprise at least one UV filter substance and preferably have an SPF of at least 4.

The UV photoprotective filters used are both inorganic pigments, for example titanium dioxide or zinc oxide, and also organic compounds, in most cases aromatic substances with a defined pi electron system.

Sun protection compositions come in various forms. Those which are particularly popular are water resistant products in which the filter substances are not immediately washed off when the skin and/or the hair comes into contact with water. This washing-off is reduced or suppressed by the presence of further substances in the photoprotective compositions.

In one embodiment, the present invention relates to cosmetic photoprotective preparations selected from the group of cosmetic and dermaological photoprotective compositions comprising the polyurethanes PU described above. Within the context of this invention, cosmetic or dermatological photoprotective compositions are understood as meaning cosmetic or dermatological preparations which, besides the polyurethanes PU, comprise at least one, preferably two or more, UV filter substances. The cosmetic or dermatological photoprotective compositions of this invention preferably have a sun protection factor (SPF) of at least 4 (determined by the COLIPA method, see below).

Sun milks and creams are preferably produced as oil-in-water (O/W) emulsions and as water-in-oil (W/O) emulsions. The properties of the preparations are very different depending on the type of emulsion: O/W emulsions can be spread easily on the skin; mostly, they absorb rapidly into the skin and almost always can be washed off again easily with water. W/O emulsions are more difficult to rub in; they grease the skin to a greater extent and are thereby somewhat stickier in effect, but on the other hand better protect the skin from drying out. W/O emulsions are in most cases water-resistant. In the case of O/W emulsions, the emulsion base, the selection of suitable photoprotective substances and, if appropriate, the use of auxiliaries (e.g. polymers) determine the degree of water resistance. The bases of liquid and cream-like O/W emulsions resemble the other emulsions customary in skincare in terms of their composition. Sun milks are intended to adequately grease the skin dried out by sun, water and wind. They must not be sticky since this is perceived as particularly unpleasant in the heat and upon contact with sand.

The photoprotective agents are generally based on a carrier which comprises at least one oil phase. However, preparations based merely on water are also possible. Accordingly, oils, oil-in-water emulsions and water-in-oil emulsions, creams and pastes, lip protection stick masses or grease-free gels are contemplated.

Suitable emulsions are inter alia also O/W macroemulsions, O/W microemulsions or O/W/O emulsions with surface-coated titanium dioxide particles present in dispersed form, where the emulsions are obtainable for example by phase inversion technology, as in DE-A-197 26 121 (PIT emulsions).

Customary cosmetic auxiliaries which may be suitable as additives are e.g. (co-) emulsifiers, fats and waxes, stabilizers, further thickeners, biogenic active ingredients, film formers, fragrances, dyes, pearlizing agents, preservatives, pigments, electrolytes (e.g. magnesium sulfate) and pH regulators.

Stabilizers which can be used are metal salts of fatty acids, such as e.g. magnesium stearate, aluminum stearate and/or zinc stearate.

Biogenic active ingredients are to be understood as meaning for example plant extracts, protein hydrolysates and vitamin complexes.

Customary film formers are for example hydrocolloids such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds.

### Sun Protection Factor (SPF) and COLIPA Method

### Sun Protection Factor (SPF)

In Europe, the sun protection factor (SPF) is determined in accordance with the COLIPA standard of the European Cosmetic, Toiletry and Perfumery Association. It denotes the protection performance of sunscreen products against UVB rays. These are the main causes of sunburn. In addition, they have immunosuppressive and cell-damaging effects which, in case of chronic exposure, lead to the formation of skin cancer (basalioma, spinalioma). Various UV filters or UV filter systems/combinations are used in order to achieve optimum protection performance. If the minimum erythema dose (MED), i.e. the amount of UVB radiation which induces a just perceptible reddening (erythema), is determined, the SPF arises according to the following formula:

$$LSF = \frac{MED auf gesch \ddot{u} tzter Haut}{MED auf ungesch \ddot{u} tzter Haut} = \frac{{MED}_{g}}{{MED}_{u}}$$

$$\begin{matrix}SPF = MED on protected skin \\ MED on unprotected skin \\ and the {subscript }^{‵} {g}^{′} {to }^{‵} {p}^{′}\end{matrix}$$

The test procedure starts with the determination of the individual UV sensitivity of the subjects by exposing unprotected areas of skin on the back to radiation. Radiation sources are solar simulators, in most cases equipped with xenon lamps: they produce a radiation that is solar-like in its spectrum, but which is of higher intensity and thereby permits shorter irradiation times. About 20 hours following irradiation with different intensity, the MEDᵤ is determined by visually assessing the erythemas in six areas of skin. To actually determine the SPF, test areas are again marked on the backs of the subjects for the sun protection preparations to be applied. Each of these product areas is compared with an adjacent control area with untreated skin. The irradiation intensity is set depending on skin sensitivity and expected sun protection factor. After about 20 hours, MEDᵤ and MEDₚ are ascertained visually. The resulting sun protection factors indicate the average extension of the individual time span until an erythema arises which has been achieved through the use of the photoprotective preparation.

The COLIPA method is the method known to the person skilled in the art and valid throughout Europe since 1997 for determining the sun protection factor (UVB protection) of sunscreen products. The test method is standardized: the irradiation spectrum and the starting output of the sun simulator provided for the test are defined exactly. Additionally, the application amount and the nature of product application are precisely prescribed. The test method is independent of the skin type and the age of the test persons. Using these precise settings for the COLIPA method the test of sunscreen products can be carried out according to the appropriate statistical position with only ten subjects. The COLIPA method is a process which produces reproducible results with high reliability.

### UV Filter Substances

In one embodiment of the invention, the preparations according to the invention comprise oil-soluble and/or water-soluble UVA and/or UVB filters besides the polyurethanes PU.

These sun protection preparations advantageously comprise substances which in the UVB region absorb UV radiation and substances which absorb UV radiation in the UVA region, the total amount of the filter substances being e.g. 0.1 to 50% by weight, preferably 0.5 to 30% by weight, in particular 1 to 15% by weight, based on the total weight of the preparations, in order to provide cosmetic preparations which protect the skin against the entire range of ultraviolet radiation.

The majority of the photoprotective agents in the cosmetic or dermatological preparations serving to protect the human epidermis consists of compounds which absorb UV light in the UV-B region. For example, the fraction of the UV-A absorbers to be used according to the invention is 10 to 90% by weight, preferably 20 to 50% by weight, based on the total amount of substances absorbing UV-B and UV-A.

A further embodiment of the invention is in particular sunscreen formulations comprising the polyurethanes PU with a balanced UV-A balance, as described under EP 1291640 A1 or corresponding to the German Industry Standard (DIN) 67502 (quality standard for protection against photoinduced skin aging) are prepared. Reference is hereby made to these passages in their entirety.

The UVB filters may be oil-soluble, water-soluble or pigmentary. Advantageous UVB filter substances are e.g.:
benzimidazolesulfonic acid derivatives, such as e.g. 2-phenylbenzimidazole-5-sulfonic acid and salts thereof
benzotriazole derivatives such as e.g. 2,2′-methylenebis(6-(2H-benzotriazol-2-yl)-4-(1,1,3,3-tetramethylbutyl)phenol) (Tinosorb® M)
4-aminobenzoic acid derivatives, preferably 2-ethylhexyl 4-(dimethylamino)-benzoate, amyl 4-(dimethylamino)benzoate;
esters of benzalmalonic acid, preferably di(2-ethylhexyl) 4-methoxybenzalmalonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
methylidenecamphor derivatives, preferably 4-methyl benzylidenecamphor, benzylidenecamphor;
triazine derivatives, preferably tris(2-ethylhexyl) 4,4′,4″-(1,3,5-triazine-2,4,6-triylimino)trisbenzoate [INCI: Diethylhexyl Butamido Triazine, UVA-Sorb® HEB (Sigma 3V)] and 2,4,6-tris[anilino(p-carbo-2′-ethyl-1′-hexyloxy)]-1,3,5-triazine [INCI: Octyl Triazone, UVINUL®T 150 (BASF)].

Water-soluble UVB filter substances to be used advantageously are e.g.:
sulfonic acid derivatives of 3-benzylidenecamphor, such as e.g. 4-(2-oxo-3-bornylidenemethyl)benzenesulfonic acid, 2-methyl-5-(2-oxo-3-bornylidenemethyl)sulfonic acid and salts thereof.

UVA filters to be used advantageously are e.g.:
1,4-phenylenedimethinecamphorsulfonic acid derivatives, such as e.g. 3,3′-(1,4-phenylenedimethine)bis(7,7-dimethyl-2-oxobicyclo[2.2.1]heptane-I-methanesulfonic acid and its salts
1,3,5-triazine derivatives, such as 2,4-bis{[(2-ethylhexyloxy)-2-hydroxy)phenyl}-6-(4-methoxyphenyl)-1,3,5)-triazine (e.g. Tinosorb®S (Ciba))
dibenzoylmethane derivatives, preferably 4-isopropyldibenzoylmethane, 4-(tert-butyl)-4′-methoxydibenzoyl methane
benzoxazole derivatives, for example 2,4-bis[4-[5-(1,1-dimethyl-propyl)benzoxazol-2-yl]phenylimino]-6-[(2-ethylhexyl)imino]-1,3,5-triazine (CAS No. 288254-1 6-0, Uvasorb®K2A (3V Sigma))
hydroxybenzophenones, for example hexyl 2-(4′-diethylamino-2′-hydroxybenzoyl)-benzoate (also: aminobenzophenone) (Uvinul®A Plus (BASF)).

In addition, according to the invention, it may, if appropriate, be advantageous to provide preparations with further UVA and/or UVB filters, for example certain salicylic acid derivatives, such as 4-isopropylbenzyl salicylate, 2-ethylhexyl salicylate, octyl salicylate, homomethyl salicylate. The total amount of salicylic acid derivatives in the cosmetic or dermatological preparations according to the invention is advantageously selected from the range 0.1-15.0, preferably 0.3-10.0% by weight, based on the total weight of the preparations. A further photoprotective filter to be used advantageously according to the invention is ethylhexyl 2-cyano-3,3-diphenylacrylate (octocrylene, commercially available for example as Uvinul®N 539 (BASF)).

The following table lists some photoprotective filters suitable for use in the preparations according to the invention:

| | | CAS No. |
|---|---|---|
| No. | Substance | (= acid) |
| 1 | 4-Aminobenzoic acid | 150-13-0 |
| 2 | 3-(4′-Trimethylammonium)benzylidenebornan-2-one methyl- | 52793-97-2 |
| | sulfate | |
| 3 | 3,3,5-Trimethylcyclohexyl salicylate (homosalate) | 118-56-9 |
| 4 | 2-Hydroxy-4-methoxybenzophenone (oxybenzone) | 131-57-7 |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl-2- | 90457-82-2 |
| | oxobicyclo[2.2.1]heptane-1-methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-ethylhexyl 4-methoxycinnamate | 5466-77-3 |
| 12 | 2-Hydroxy-4-methoxybenzophenone-5-sulfonic acid | 4065-45-6 |
| | (sulisobenzonum) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone | 1641-17-4 |
| | (mexenone) | |
| 24 | Triethanolamine salicylate | 2174-16-5 |
| 25 | Dimethoxyphenylglyoxalic acid or: | 4732-70-1 |
| | sodium 3,4-dimethoxyphenyl glyoxalate | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3- | 103597-45-1 |
| | tetramethylbutyl)phenol] | |
| 30 | 2,2′-(1,4-Phenylene)bis-1H-benzimidazole-4,6- | 180898-37-7 |
| | disulfonic acid, Na salt | |
| 31 | 2,4-bis[4-(2-Ethylhexyloxy)-2-hydroxy]phenyl- | 187393-00-6 |
| | 6-(4-methoxyphenyl)-(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone 5,5′- | 3121-60-6 |
| | disodium sulfonate | |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Polymeric or polymer-bonded filter substances, such as, for example, Parsol®SLX can also be used according to the invention.

Photoprotective agents suitable for use in the preparations according to the invention are also the compounds specified in EP-A 1 084 696 in paragraphs [0036] to [0053], to which reference is made at this point in their entirety. Of suitability for the use according to the invention are all UV photoprotective filters which are specified in Appendix 7 (to §3b) of the German Cosmetics Ordinance under “Ultraviolet filters for cosmetic compositions”.

Metal oxides such as titanium dioxide or zinc oxide are widespread in sun protection compositions. Their effect is essentially based on reflection, scattering and absorption of the harmful UV radiation and depends essentially on the primary particle size of the metal oxides. In a particularly preferred embodiment of the invention, the cosmetic or dermatological preparations according to the invention comprise inorganic pigments based on metal oxides and/or other sparingly water-soluble or insoluble metal compounds, selected from the group of the oxides of zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, mixtures of such oxides, and dopings of the oxides with foreign metal fractions and/or the coating of metal oxides with other metal oxides. Particular preference is given to pigments based on ZnO and/or TiO₂.

Accordingly, particularly preferred embodiments of the invention are cosmetic or dermatological photoprotective preparations which comprise polyurethanes PU and zinc oxide and/or titanium dioxide as inorganic UV photoprotective filters.

In a further embodiment, the viscosity of water-comprising preparations according to the invention which comprise polyurethane PU and at least one photoprotective agent, in particular zinc oxide or titanium dioxide, is increased by virtue of the presence of the polyurethanes PU in the preparation compared to preparations which comprise only photoprotective agents or only polyurethanes PU. Here, the order in which polyurethanes PU and photoprotective agents are added is unimportant.

The inorganic pigments here may be present in coated form. This coating can consist for example in providing the pigments in a manner known per se, as described in DE-A-33 14 742, with a thin hydrophobic layer.

Besides the polyurethanes PU and a cosmetically acceptable carrier, particularly preferred cosmetic photoprotective preparations comprise UV photoprotective agents based on zinc oxide which are commercially available as Z-Cote®, Z-Cote®HP1 or Z-Cote®MAX.

Besides the polyurethanes PU, particularly preferred cosmetic photoprotective preparations comprise UV photoprotective agents based on titanium dioxide which are commercially available as T-Lite®, T-Lite™SF, T-Lite™SF-S or T-Lite™MAX. The list of specified UV photoprotective filters which can be used in the preparations according to the invention is not exhaustive.

One embodiment of the invention is cosmetic preparations which comprise polyurethanes PU and at least one water-soluble UV filter.

A further embodiment of the invention is cosmetic preparations which comprise polyurethane PU and at least one ionogenic UV filter.

For example, 2-phenylbenzimidazole-5-sulfonic acid (Eusolex®232) can ideally be stably formulated up to the maximum permissible concentration of 8% by weight, which was hitherto possible only to an unsatisfactory degree. Furthermore, combinations of different UV filters can also be stably formulated in the presence of polyurethane PU, in which case a good skin feel is observed compared to other thickener systems.

### Preparations for Decorative Cosmetics

The invention also relates to cosmetic preparations, preferably in liquid or pasty form, for use on the skin, on semimucosa, on mucosa and in particular on keratinic material such as hair, eyelashes and eyebrows, in particular for the shaping, decoration, coloring, beautifying of the same, and also for the care of the skin and of the skin appendages. Preparations of this type are used for example for the shaping and coloring, in particular of the eyelashes and of the hair—such a preparation is then referred to as “mascara”.

In principle, the preparations according to the invention can be used, with suitable adjustment and coloration, also as make-up, concealer, camouflage, eyeshadows, eyeliners, lipliners, blusher, lip blush, lip gloss, sun protection composition, sun block, temporary tattoo, colored effect sunscreen for surfers and the like.

A preferred embodiment of the present invention is thus cosmetic preparations for decorative cosmetics. These are preferably preparations which are used for making up the skin and comprise at least one decorative constituent such as dyes, colored pigments, pearlescent pigments. Preparations of this type are also referred to as foundations or face make-up. In the field of cosmetic preparations, both soluble colorants (within the context of the present invention also referred to as dyes) and insoluble colorants (within the context of the present invention also referred to as (colored) pigments) are approved for coloring the product or for coloring the object to be treated (skin, hair, lips). All decorative bodycare compositions comprise a greater or lesser fraction of dyes, colored pigments and/or pearlescent pigments since the color change in facial skin, the eye region, the lips and/or the nails is the main purpose of these products. In addition, these products usually additionally comprise further ingredients with a skincare or skin-protective effect. In general, the application of cosmetic make-up preparations should emphasize the features and individuality of a person and serve to emphasize personal attractiveness and conceal any blemishes. The making-up process usually takes place in several steps. Firstly, a liquid base (foundation or face make-up) is applied, which evens out the skin tone and conceals irregularities in the skin (such as e.g. skin blemishes or circles around the eyes). It is intended to give the skin a natural and radiant complexion and also a youthful appearance. More severe unevenness or reddening can be concealed by extra means, using a skin-colored (concealing) stick or liquid concealer. Usually, loose or compact powder is then applied in order to matte the facial skin. The cheeks are then tinted using blusher and the eyes are made up with eyeshadows, kohl, eyeliner and/or mascara. Face make-up preparations can advantageously also comprise silicone oils or silicone derivatives because these components contribute to making the formulations very easy to spread on the skin and uniform and also to giving the skin a grease-free, soft shine and in so doing leaving behind a velvety skin feel.

In one embodiment of the invention, the cosmetic preparations are of the W/O type. In a preferred embodiment of the invention, the cosmetic preparations are of the O/W emulsion type.

The preparations according to the invention are suitable in particular for concealing skin blemishes and/or circles around the eyes, for concealing small wrinkles, for achieving an even, natural and radiant complexion and a youthful appearance. In this connection, the effect achieved in each case is surprisingly long-lasting. For use, the preparations according to the invention are applied to the skin in an adequate amount in the manner customary for cosmetics. They can have the customary composition and serve to care for the skin and as a make-up product in decorative cosmetics.

The pigments used in the preparations according to the invention for the decorative cosmetics may be inorganic or organic. Preferred pigments are described in DE 10 2006 028 549 A1, sections [0018] to [0026], to which reference is hereby made in its entirety.

It is also advantageous within the context of the present invention if the preparation according to the invention comprises one or more dyes. The dyes may either be synthetic or of natural origin. The cosmetic preparations according to the invention can also advantageously comprise fillers which e.g. further improve the sensory and cosmetic properties of the preparations and bring about or intensify for example a velvety or silky skin feel.

Advantageous fillers within the context of the present invention are starch and starch derivatives (such as e.g. tapioca starch, distarch phosphate, aluminum starch or sodium starch octenylsuccinate and the like), pigments which have neither primarily a UV filter effect nor a coloring effect (such as e.g. boron nitride etc.) and/or Aerosile® (CAS No. 7631-86-9), and also lauroyl lysine, polymethylsilesquioxane, polymethyl methacrylates, polymethyl methacrylate crosspolymer, nylon, talc, coated talc, e.g. with dimethicone and trimethylsiloxysilicates, mica, silica.

The preparations according to the invention can also advantageously comprise one or more further (silicone) emulsifiers, preferably emulsifiers with a HLB value of less than or equal to 8 if the preparations are water-in-silione oil emulsions (W/S emulsions).

Preferred silicone emulsifiers are described in DE 10 2006 028 549 A1, sections to [0037], to which reference is hereby made in its entirety.

The cosmetic preparations according to the invention can also advantageously comprise one or more interface-active polyethers, particularly if the preparations are in the form of W/S emulsions. Preferred interface-active polyethers are described in DE 10 2006 028 549 A1, section [0038], to which reference is hereby made in its entirety. The total amount of the interface-active polyethers in the finished cosmetic preparations is advantageously selected from the range from 0.1 to 30% by weight, preferably 0.25 to 5.0% by weight, in particular 0.75 to 3.5% by weight, in each case based on the total weight of the preparations.

The preparation according to the invention can also be present in the form of a water-in-oil emulsion (W/O emulsion). In this case, the emulsifiers which are specified in DE 10 2006 028 549 A1, section [0041], are preferred according to the invention.

In a particularly preferred embodiment, the oil phase has a content of cyclic and/or linear silicone oils, or cyclic and/or linear silicone oils are used as the sole oil components.

In addition, the oil phase can have a content of dialkyl carbonates, e.g. dicaprylyl carbonate is advantageous, for example that available under the tradename Cetiol®CC (Cognis). In addition, the oil phase can comprise triglycerides such as caprylic/capric triglycerides, dialkyl ethers such as dicaprylyl ether, natural oils such as avocado oil, sesame oil, almond oil, soybean oil, apricot oil and hydrocarbons, linear or branched. The oil phase of the preparations according to the invention can also advantageously comprise wax components, in particular waxes whose melting point is between 30 and 45° C., particularly preferably between 30 and 40° C. Preferred waxes are described in DE 10 2006 028 549 A1, section [0049], to which reference is hereby made in its entirety.

The preparations according to the invention for decorative cosmetics preferably further comprise preservatives, complexing agents, antioxidants, active ingredients as described in DE 10 2006 028 549 A1, section [0063].

The water phase of the preparations according to the invention for decorative cosmetics can further active ingredients as described in DE 10 2006 028 549 A1, section [0066]. However, it is possible to dispense with the thickeners mentioned therein on account of the presence of the thickened polyurethanes PU.

It is also advantageous within the context of the present invention if the preparation comprises one or more film formers or polymers, in particular in order to achieve a perceptible tightening effect on the skin which can be brought about by their film-forming properties. At the same time, the film formers serve for fixing the pigments on the skin, in particular in order to achieve a long-lasting effect and a transfer resistance. Polymers suitable according to the invention and their contents are described in DE 10 2006 028 549 A1, sections [0073] to [0076].

Besides the water forming the continuous phase of the emulsion, and the polyurethane PU, the most important constituents of the preparation of the O/W type preferred according to the invention are a wax component, a polyhydric alcohol and a film former system. The wax component consists of at least one wax and optionally additionally at least one fat and/or oil, which may in each case be of vegetable, animal, mineral or synthetic origin. In addition, at least one emulsifier and at least one coemulsifier may be present in order to facilitate the processing of the wax component to an emulsion. In order to obtain an esthetically particularly satisfactory result, the wax component can additionally comprise a polyvinylpyrrolidone copolymer. The wax component gives the mass the desired consistency and makes the composition water-resistant and tear-resistant. For this purpose, the wax component can be composed of fat-, oil- and wax-like raw materials, which may be liquid, paste-like or solid at temperatures from 21° C. to 25° C. Preferably, for establishing the optimum consistency, a combination of at least of one wax, and at least one oil is used. Preferred waxes are described in DE 10 2005 033 520 A1, sections [0017] and [0018], to which reference is hereby made in its entirety. Preferred oils and fats are described in DE 10 2005 033 520 A1, section [0019], to which reference is hereby made in its entirety.

Preference is given to using mixtures of waxes, oils and fats, the substances listed in each case being, used in amounts such that the desired properties, such as structure and viscosity, are achieved. The amounts and mixtures to be used in each case are known to the person skilled in the art and therefore require no further explanation. Preferred amounts of the waxes, fats and oils are moreover described in DE 10 2005 033 520 A1, sections [0022] and [0023], to which reference is hereby made in its entirety.

Polyhydric alcohols suitable according to the invention are described in DE 10 2005 033 520 A1, section [0026], to which reference is hereby made in its entirety.

The film formers suitable for the preparations according to the invention for the decorative cosmetics and their amounts are described in DE 10 2005 033 520 A1, sections [0027] to [0030], to which reference is hereby made in its entirety.

The preparations according to the invention for decorative cosmetics can comprise further gel formers as well as the polyurethanes PU. Suitable further gel formers are described in DE 10 2005 033 520 A1, section [0032], to which reference is hereby made in its entirety.

Shampoos, Conditioners and Cleansing Products

A preferred embodiment of the invention is shampoos and cosmetic cleansing compositions comprising the polyurethanes PU. Additional requirements may be placed on shampoos and cosmetic cleansing compositions depending on hair quality or scalp problem. The mode of action of the preferred types of shampoos with the most important additional effects or most important special objectives is described below.

According to the invention, preference is given for example to shampoos for normal or rapidly greasing or damaged hair, antidandruff shampoos, baby shampoos and 2-in-1 shampoos (i.e. shampoo and rinse in one).

Shampoos according to the invention for normal hair: hair washing is intended to free hair and scalp from skin sebum formed in sebaceous glands, the inorganic salts emerging from sweat glands with water, amino acids, urea and lactic acid, shed skin particles, environmental grime, odors and, if appropriate, residues of hair cosmetic treatments. Normal hair means short to shoulder-length hair which is only slightly damaged. Accordingly, the fraction of conditioning auxiliaries should be optimized to this type of hair.

Shampoos according to the invention for rapidly greasing hair: increased sebum production by the sebaceous glands on the scalp leads to a straggly, unattractive hair style just 1-2 days after hair washing. Oil- and wax-like skin sebum constituents weigh down the hair and reduce the friction from hair to hair and thereby reduce the hold of the hair style. The actual hair cosmetic problem in the case of rapidly greasing hair is thus the premature collapse of voluminous hair styles. In order to avoid this, it is necessary to prevent the hair surface becoming weighed down and too smooth and supple. This is preferably achieved by the surfactant base of washing raw materials that wash well and are marked by particularly low substantivity. Additional care substances which would add to the skin sebum, such as refatting substances are not used in shampoos for rapidly greasing hair, or are used only with the greatest of care. Voluminizing shampoos according to the invention for fine hair can be formulated in a compatible way.

Shampoos according to the invention for dry, stressed (damaged) hair: the structure of the hair is changed in the course of hair growth as a result of mechanical influences such as combing, brushing and primarily back-combing (combing against the direction of growth), as a result of the effect of UV radiation or visible light and as a result of cosmetic treatments, such as permanent waving, bleaching or coloring.

The scaly layer of the hair has an increasingly stressed appearance from the root to the end; in extreme cases, it is completely worn away at the end and the hair ends are split (split ends). Damaged hair can in principle no longer be returned to the state of healthy hair regrowth. It is possible, however, to come very close to this ideal state as regards feel, shine and combability by using shampoos according to the invention containing, if appropriate, high fractions of care substances (conditioning agents). An even better hair-conditioning effect than with a shampoo is achieved with a haircare composition according to the invention for example in the form of a treatment with a rinsing or treatment composition after washing the hair. Rinsing or treatment compositions for hair which comprise polymers according to the invention are likewise in accordance with the invention.

2-in-1 shampoos according to the invention are particularly care-intensive shampoos in which, through the conception as “shampoo and rinse in one”, the additional care benefit is placed equally alongside the basic cleaning benefit. 2-in-one compositions according to the invention comprise increased amounts of conditioning agents. Antidandruff shampoos: compared to antidandruff hair tonics, antidandruff shampoos according to the invention have the advantage that through corresponding active ingredients to combat dandruff attack, they not only reduce the formation of newly visible flakes, and in the case of long-term use, prevent it, but remove flakes also already shed with hair washing. After rinsing out the wash liquor, however, only a small, but adequate fraction of the active ingredients remains on scalp and hair. There are various antidandruff active ingredients which can be incorporated into the shampoo compositions according to the invention, such as e.g. zinc pyrithione, ketoconazole, elubiol, clotrimazole, climbazole or piroctone olamine. Additionally, these substances have an effect to normalize shedding.

The basis of antidandruff shampoos corresponds predominantly to the formulation of shampoos for normal hair with a good cleaning effect.

Baby shampoos: in a preferred embodiment of the invention, the shampoo preparations according to the invention are baby shampoos. These are optimally skin- and mucosa-compatible, e.g. through selection of the surfactant mixture and a reduced salt content. Combinations of washing raw materials with very good skin compatibility form the basis of these shampoos. Additional substances to further improve the skin and mucosa compatibility and the care properties are advantageously added, such as e.g. nonionic surfactants, protein hydrolysates and panthenol or bisabolol. All necessary raw materials and auxiliaries, such as preservatives, perfume oils, dyes etc., are selected from the aspect of high compatibility and mildness.

Shampoos for dry scalp: in a further preferred embodiment of the invention, the shampoo preparations according to the invention are shampoos for dry scalp. The primary aim of these shampoos is to prevent the scalp from drying out since dry scalp can lead to itching, redness and inflammation. As is also the case with the baby shampoos, combinations of washing raw materials with very good skin compatibility form the basis of these shampoos. Additionally, if appropriate, refatting agents and humectants, such as e.g. glycerol or urea, can be used.

Preferred shampoos and cosmetic cleaning compositions comprise anionic surfactants. Further preferred shampoos and cosmetic cleaning compositions comprise combinations of anionic and ampholytic surfactants. Further preferred shampoos and cosmetic cleaning compositions comprise combinations of anionic and zwitterionic surfactants. Further preferred shampoos and cosmetic cleaning compositions comprise combinations of anionic and nonionic surfactants.

Suitable surfactants of all types are those already described above under “Surfactants”.

Preferred anionic surfactants are alkyl sulfates, alkykl polyglycol ether sulfates and ether carboxylic acid salts having 10 to 18 carbon atoms in the alkyl group and up to 12 glycol ether groups in the molecule and sulfosuccinic acid mono- and dialkyl esters having 8 to 18 carbon atoms in the alkyl group and sulfosuccinic acid monoalkyl polyoxyethyl esters having 8 to 18 carbon atoms in the alkyl group and 1 to 6 oxyethyl groups. Particularly preferred anionic surfactants are the alkali metal or ammonium salts of lauryl ether sulfate with a degree of ethoxylation of from 2 to 4 EO units.

A preferred zwitterionic surfactant is the fatty acid amide derivative known under the INCI name Cocamidopropyl Betaine.

Particularly preferred ampholytic surfactants are N-cocosalkylaminopropionate, cocosacylaminoethylaminopropionate and the C12-C12-acylsarcosine.

Preferred nonionic surfactants have proven to be the alkylene oxide addition products onto saturated linear fatty alcohols and fatty acids having in each case 2 to 30 mol of ethylene oxide per mole of fatty alcohol or fatty acid. Preparations with exceptional properties are likewise obtained if they comprise fatty acid esters of ethoxylated glycerol as nonionic surfactants.

The shampoo compositions according to the invention can also be present as shampoo concentrates with increased surfactant contents of 20-30% by weight. They are based on specific washing raw material combinations and consistency regulators which ensure the good spreadability and the spontaneous foaming capacity even of a small application amount. A particular advantage is for example the possibility of achieving the productivity of 200 ml of shampoo with a 100 ml bottle.

### Presentation

The preparations according to the invention can be present for example as preparations that can be sprayed from aerosol containers, squeezable bottles or through a pump, spray or foaming device, but also in the form of a composition that can be applied from standard bottles and containers. Suitable propellants for cosmetic or dermatological preparations that can be sprayed from aerosol containers within the context of the present invention are the customary known readily volatile, liquefied propellants, for example dimethyl ether, hydrocarbons (propane, butane, isobutane), which can be used on their own or in a mixture with one another. Compressed air, nitrogen, nitrogen dioxide or carbon dioxide or mixtures of these substances can also be used advantageously.

The preparations according to the invention can be prepared in the customary manner by mixing the individual constituents. The active ingredients of the preparations according to the invention or else the premixed constituents of the preparations according to the invention can be added in the mixing process. The pH of the preparations can be adjusted in a known manner by adding acids or bases, preferably by adding buffer mixtures, e.g. based on citric acid/citrate or phosphoric acid/phosphate buffer mixtures. Preferably, the pH is below 10, e.g. in the range 2-7, in particular in the range 3-5.

Preferred Shampoo Formulations Comprise

a) 0.05 to 10% by weight of at least one polyurethane PU,
b) 25 to 94.95% by weight of water,
c) 5 to 50% by weight of surfactants,
c) 0 to 5% by weight of a conditioning agent,
d) 0 to 10% by weight of further cosmetic constituents.

In a further embodiment, by using polyurethanes PU, it is also possible to prepare surfactant-reduced formulations with less than 10% by weight of surfactant, based on the preparation, in a viscosity adequate for the preparation. In particular, polyurethanes PU are adjusted for establishing the desired viscosity in those preparations which comprise at least 0.1 salt by weight and from 0.1 to 10% by weight, preferably less than 10% by weight, of surfactant.

In the shampoos and cosmetic cleaning compositions, all anionic, neutral, amphoteric or cationic surfactants customary in shampoos and cosmetic cleaning compositions can be used. Suitable surfactants have been specified above. Particular preference is given to shampoos and cosmetic cleaning compositions with a surfactant content of more than 10% by weight.

In the shampoo formulations, customary conditioning agents can be used to achieve certain effects. These include, for example, cationic polymers with the name Polyquaternium in accordance with INCI, in particular copolymers of vinylpyrrolidone/N-vinylimidazolium salts (Luviquat®FC, Luviquat®HM, Luviquat®MS, Luviquat®Care), copolymers of N-vinylpyrrolidone/dimethylaminoethyl methacrylate, quaternized with diethyl sulfate (Luviquat®PQ 11), copolymers of N-vinylcaprolactam/N-vinylpyrrolidone/N-vinylimidazolium salts (Luviquat®Hold); cationic cellulose derivatives (Polyquaternium-4 and -10), acrylamide copolymers (Polyquaternium-7).

Advantageous conditioning agents are for example the compounds referred to in accordance with INCI as Polyquaternium (in particular Polyquaternium-1 to Polyquaternium-87). The table below gives a nonexhaustive overview of conditioning agents which are used in combination with the polymers according to the invention:

| | | | Example |
|---|---|---|---|
| INCI name | CAS number | Polymer type | (tradename) |
| Polyquaternium-2 | CAS 63451-27-4 | Urea, N,N′,bis [3- | Mirapol ® A- |
| | | (dimethylamino)propyl]polymer with | 15 |
| | | 1,1′-oxybis(2-chloroethane) | |
| Polyquaternium-5 | CAS 26006-22-4 | Acrylamide, β- | |
| | | methacryloxyethyltriethylammonium | |
| | | methosulfate | |
| Polyquaternium-6 | CAS 26062-79-3 | N,N-Dimethyl-N-2-propenyl-2- | Merquat ® |
| | | propenaminium chloride | 100 |
| | | (PolyDADMAC) | |
| Polyquaternium-7 | CAS 26590-05-6 | N,N-Dimethyl-N-2-propenyl-2- | Merquat ® S |
| | | propenaminium chloride, 2- | |
| | | propenamide | |
| Polyquaternium- | CAS 53568-66-4, | Quaternary ammonium salt of | Celquat ® |
| 10 | 55353-19-0, | hydroxyethylcellulose | SC-230M, |
| | 54351-50-7, | | Polymer JR |
| | 68610-92-4, | | 400 |
| | 81859-24-7 | | |
| Polyquaternium- | CAS 53633-54-8 | Vinylpyrrolidone/dimethylaminoethyl | Gafquat ® |
| 11 | | methacrylate copolymer/diethyl | 755N |
| | | sulfate reaction product | |
| Polyquaternium- | CAS 29297-55-0 | Vinylpyrrolidone/vinylimidazolinium | Luviquat ® |
| 16 | | methochloride copolymer | HM552 |
| Polyquaternium- | CAS 90624-75-2 | | Mirapol ® |
| 17 | | | AD-1 |
| Polyquaternium- | CAS 110736-85-1 | Quaternized water-soluble | |
| 19 | | polyvinyl alcohol | |
| Polyquaternium- | CAS 110736-86-2 | Water-dispersible quaternized | |
| 20 | | polyvinyl octadecyl ether | |
| Polyquaternium- | | Polysiloxane | Abil ® B |
| 21 | | polydimethyldimethylammonium | 9905 |
| | | acetate copolymer | |
| Polyquaternium- | CAS 53694-17-0 | Dimethyldiallylammonium | Merquat ® |
| 22 | | chloride/acrylic acid copolymer | 280 |
| Polyquaternium- | CAS 107897-23-5 | Polymeric quaternary ammonium | Quartisoft ® |
| 24 | | salt of hydroxyethylcellulose | LM-200 |
| Polyquaternium- | CAS 131954-48-8 | Vinylpyrrolidone/methacrylamido- | Gafquat ® |
| 28 | | propyltrimethylammonium chloride | HS-100 |
| | | copolymer | |
| Polyquaternium- | CAS 92091-36-6, | Chitosan which has been reacted | Lexquat ® |
| 29 | 148880-30-2 | with propylene oxide and | CH |
| | | quaternized with epichlorohydrin | |
| Polyquaternium- | CAS 136505-02- | Polymeric, quaternary ammonium | Hypan ® QT |
| 31 | 7, 139767-67-7 | salt which is prepared by reacting | 100 |
| | | DMAPA acrylate/acrylic | |
| | | acid/acrylonitrogens copolymer | |
| | | and diethyl sulfate | |
| Polyquaternium- | CAS 35429-19-7 | N,N,N-Trimethyl-2-([82-methyl-1- | |
| 32 | | oxo-2-propenyl)oxy]ethanaminium | |
| | | chloride, polymer with 2- | |
| | | propenamide | |
| Polyquaternium- | CAS 26161-33-1 | | |
| 37 | | | |
| Polyquaternium- | | Copolymeric quaternary | |
| 44 | | ammonium salt of vinylpyrrolidone | |
| | | and quaternized imidazoline | |
| Polyquaternium- | | polymeric quaternary ammonium | SoftCAT ® |
| 67 | | salt of hydroxyethyl cellulose | |
| | | reacted with a trimethyl | |
| | | ammonium substituted epoxide | |
| | | and a lauryl dimethyl ammonium | |
| | | substituted epoxide | |
| Polyquaternium- | | | Polycare ® |
| 74 | | | Boost |
| Polyquaternium- | | | Luviquat ® |
| 87 | | | Sensation |

In addition, it is possible to use protein hydrolysates, and also conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and amino-functional silicone compounds such as amodimethicones (CTFA).

The shampoos and cosmetic cleaning compositions according to the invention preferably comprise salts. Shampoos and cosmetic cleaning compositions according to the invention based on anionic surfactants preferably comprise sodium chloride. It is a particular advantage of the present invention that the viscosity and the optical appearance of the shampoos and cosmetic cleaning compositions according to the invention remain stable over a long period even with concentrations of sodium chloride of more than 0.2% by weight and/or surfactant concentrations of more than 10% by weight.

In a further preferred embodiment of the invention, the shampoos and cosmetic cleaning compositions moreover comprise at least one constituent from the group of the water-insoluble oil components, the vitamins, the provitamins, the protein hydrolysates, the plant extracts, the UV filters, the amino acids, the water-insoluble silicones, the water-soluble silicones and/or the amodimethicones.

The total amount of oil and fat components in the shampoo preparations according to the invention is usually 6-45% by weight, based on the preparation. Amounts of 10-35% by weight are preferred according to the invention.

Vitamins, provitamins and vitamin precursors preferred according to the invention, and derivatives thereof, are to be understood as meaning those representatives which are usually assigned to groups A, B, C, E, F and H. Preferably, the preparations used according to the invention comprise vitamins, provitamins and vitamin precursors from groups A, E, F and H. Two or more vitamins and vitamin precursors can of course also be present at the same time. The total use amount of the vitamins, provitamins, vitamin precursors and derivatives thereof in the preparations according to the invention is—based on the total weight of the preparation—0.01 to 5% by weight, preferably 0.02 to 4% by weight and in particular 0.05 to 3% by weight.

Preferably, the shampoos and cosmetic cleaning compositions according to the invention also comprise protein hydrolysates. Within the context of the invention, protein hydrolysates are understood as meaning protein hydrolysates and/or amino acids and derivatives thereof. Protein hydrolysates are product mixtures which are obtained by acidically, basically or enzymatically catalyzed degradation of proteins. According to the invention, the term protein hydrolysates is also understood as meaning total hydrolysates and individual amino acids and derivatives thereof, and also mixtures of different amino acids. Furthermore, according to the invention, polymers composed of amino acids and amino acid derivatives are understood under the term protein hydrolysates. The latter include for example polyalanine, polyasparagine, polyserine etc. Further examples of compounds that can be used according to the invention are L-alanyl-L-proline, polyglycine, glycyl-L-glutamine or D/L-methionine-S-methylsulfonium chloride. According to the invention, it is also of course possible to use P-amino acids and derivatives thereof, such as P-alanine, anthranilic acid or hippuric acid. The molecular weight of the protein hydrolysates that can be used according to the invention is between 75, the molecular weight for glycine, and 200 000, preferably the molecular weight is 75 to 50 000 and very particularly preferably 75 to 20 000 daltons.

Preferred protein hydrolysates are described in DE 10 2006 032 505 A1, paragraphs to [0079], the preferred amounts of which are described ibid in paragraph [0080]. Reference is hereby made to these passages in their entirety.

According to the invention, preference is given to surfactant-containing cleaning compositions, in particular shampoos which comprise plant extracts. Preferred plant extracts are described in DE 10 2006 032 505 A1, paragraphs [0081] to [0082], the preferred amounts of which are described ibid in paragraph [0086]. Reference is hereby made to these passages in their entirety.

According to the invention, preference is given to surfactant-containing cleaning compositions, in particular shampoos, which comprise UV photoprotective agents (UV filters). The effect of the preparations can be increased through UV filters. The UV filters suitable according to the invention are not subject to any general limitations as regards their structure and their physical properties. Rather, all UV filters which can be used in the cosmetics sector are suitable, the absorption maximum of which is in the UVA region (315-400 nm), in the UVB region (280-315 nm) or in the UVC region (<280 nm). UV filters with an absorption maximum in the range from about 280 to about 300 nm are particularly preferred. The UV filters suitable according to the invention can be selected for example from substituted benzophenones, p-aminobenzoic acid esters, diphenylacrylic acid esters, cinnamic acid esters, salicylic acid esters, benzimidazoles and o-aminobenzoic acid esters.

Examples of UV filters that can be used according to the invention are described in DE 10 2006 032 505 A1, paragraph [0089]. Reference is hereby made to this passage in its entirety.

According to a further preferred embodiment of the invention, the shampoos and cosmetic cleaning compositions moreover comprise at least one further water-insoluble silicone, a water-soluble silicone and/or an amino-functionalized silicone. Silicones suitable according to the invention bring about highly diverse effects. Thus, for example, they simultaneously influence the dry and wet combabilities, the feel of the dry and wet hair, and the shine. The term silicones is understood by the person skilled in the art as meaning several structures of organosilicon compounds. Preferred silicones are described in DE 10 2006 032 505 A1, paragraph [0113]. Reference is hereby made to this passage in its entirety.

According to a further preferred embodiment of the invention, the shampoos and cosmetic cleaning compositions moreover comprise at least one emulsifier. Preferred emulsifiers are described in DE 10 2006 032 505 A1, paragraph [0147], the preferred amounts of which are described ibid in paragraph [0148]. Reference is hereby made to these passages in their entirety.

According to a further preferred embodiment of the invention, the shampoos and cosmetic cleaning compositions moreover comprise at least one further polymer. Preferred plant extracts are described in DE 10 2006 032 505 A1, paragraphs [0151] to [0165], the preferred amounts of which are described ibid in paragraph [0167]. Reference is hereby made to these passages in their entirety.

The shampoos and cosmetic cleaning compositions according to the invention can also comprise organic solvents such as ethanol, propanol, isopropanol, benzyl alcohol, benzyloxyethanol, ethoxy diglycol, alkylene carbonates such as ethylene carbonate and propylene carbonate, phenoxyethanol, butanol, isobutanol, cyclohexane, cyclohexanol, hexylene glycol, ethylene carbonate, propylene glycol, polypropylene glycols, ethylene glycol monoethyl ether, ethylene glycol monobutyl ether, ethylene glycol monophenyl ether, 1-phenylethyl alcohol, 2-phenylethyl alcohol and o-methoxyphenol. Particularly preferred organic solvents are benzyl alcohol, benzyloxyethanol and propylene glycols. The amount of organic solvents in the preparations according to the invention should not exceed 5% by weight, should preferably be in the range from 0.1 to 3%, particularly preferably 0.5 to 2.5% by weight, calculated on the preparation.

Solubility promoters can be added to the preparations, particularly if oily substances as care agents and perfume oils with high lipophilic properties have been selected. Typical solubility promoters may be hydrogenated talc oils (for example Cremophor®RH). It should be noted that surfactant mixtures may also be good solubility promoters for perfume oils. Customary amounts of the solubility promoters may be in the range from 0.01 to 2% by weight, particularly 0.1 to 1% by weight, calculated on the total composition.

Further advantageous ingredients of the shampoos and cosmetic cleaning compositions and cosmetic cleaning compositions according to the invention are described in DE 10 2006 032 505 A1, paragraph [0168]. Reference is hereby made to this passage in its entirety.

Particular preference is given to shampoos and cosmetic cleaning compositions which comprise at least one polyurethane PU as thickener, at least one alkyl sulfate and/or alkyl ether sulfate (for example sodium lauryl sulfate) and at least one quaternary ammonium salt (for example cetyldimethyl-(2)-hydroxyethylammonium dihydrogenphosphate). Thanks to the use of the polyurethane PU, such preparations can be formulated as clear, transparent preparations, which is not achieved by conventional carbomers.

### Deodorant and Antiperspirant Preparations

In one preferred embodiment, the invention relates to deodorants or antiperspirants, in particular deodorant lotions and deodorant or antiperspirant sticks, based on an oil-in-water dispersion/emulsion for the application of active ingredients, in particular of water-soluble active ingredients, to the skin. By using polyurethane PU it is possible to formulate micro-, macro-, cream and spray deodorant and antiperspirant preparations.

Standard commercial deodorants and antiperspirants are in most cases formulated as sprays or as a stick; there are also roll-on preparations and creams on the market. Usually, antiperspirants are supplied in manifold product forms, with roll-ons, pump atomizers and aerosols predominating in Europe, and sticks predominating in the USA, Central America and South America. Both anhydrous and also water-containing products (hydroalcoholic formulations, emulsions) are known. The principal problem with emulsions comprising antiperspirants consists in a destabilizing effect of high contents of electrolytes, especially in preparations with low pH values. Consequently, emulsions are not storage-stable and are often subject to phenomena such as creaming or sedimentation. This applies in particular to thin-liquid emulsions with a low viscosity. At the same time, preparations of this type have to be perceived by the user as being pleasant to use.

Many stick-like antiperspirant preparations are formulated as anhydrous suspension sticks. Preparations of this type leave behind a pleasantly dry skin feel following application by the user. An effective release of the water-soluble antiperspirant active ingredients from such preparations, however, is limited (cf. Chemistry and Technology of the Cosmetics and Toiletries Industry, ed.: D. F. Williams and W. H. Schmitt, London: Blackie, 1996, 2nd edition, p. 326), and in most cases the freshness feel valued by many consumers is not achieved. The anhydrous preparations, in particular those based on volatile silicone oils, have the disadvantage that the dispersed active ingredients readily lead to visible product residues on skin and clothing. Moreover, such preparations are relatively costly since the oil components as active ingredient carriers are more expensive than water. The exertion of pressure upon application often leads to an oiling-out, which reduces the cosmetic acceptance of this preparation by the user.

The polyurethanes PU used according to the invention are advantageous as thickeners in particular in deodorant and antiperspirant preparations with high contents of electrolytes and/or acidic deodorant and antiperspirant preparations. Particularly in the case of salt contents of more than 3, preferably more than 5% by weight, based on the total preparation, the polyurethanes PU are to be used advantageously as thickeners.

Particularly at pH values in the range from 3 to 6, preferably from 4 to 5.5, the polyurethanes PU are to be used advantageously as thickeners.

In particular, by using the polyurethanes PU, it is possible to prepare stable cosmetic and/or dermatological preparations containing antiperspirant active ingredients such as, for example, aluminum chlorohydrate at a pH in the range between 3.5 and 6.

The antiperspirant active ingredient is dissolved in the preferred O/W preparations in the external, continuous aqueous phase, thus resulting in a considerably improved and more efficient release of active ingredient compared to the known anhydrous preparations, especially compared to suspension sticks and water-in-oil emulsion sticks.

The measurement of the electrical resistance of such compositions is also a suitable method for being able to quickly and easily make the distinction between an oil-in-water system and a water-in-oil system. On account of the continuos water phase, an oil-in-water system has high electrical conductivity and accordingly a low electrical resdistance. This active ingredient release can be determined indirectly very readily by measuring the electrical resistance of the particular product.

Besides the favorable active ingredient release, a formulation as oil-in-water dispersion/emulsion brings further advantages. Firstly, the composition can be washed off easily from the skin. Secondly, during or following application to the skin, a care oil-in-water cream is formed together with the skin moisture which produces a refreshing, cooling skin feel.

The deodorant and antiperspirant preparations according to the invention, in particular in stick form, comprise lipids and/or waxes in one preferred embodiment. Suitable lipids and waxes are described in DE 10 2006 021 780 A1, paragraphs to [0041], to which reference is made at this point in its entirety.

The lipid or wax component a) is preferably selected from esters of a saturated, monohydric C16-C60-alkanol and a saturated C8-C36-monocarboxylic acid, in particular cetyl behenate, stearyl behenate and C20-C40-alkyl stearate, glycerol triesters of saturated linear C12-C30-carboxylic acids, which may be hydroxylated, candelilla wax, carnauba wax, beeswax, saturated linear C14-C36-carboxylic acids, and mixtures of the aforementioned substances. Particularly preferred lipid or wax component mixtures a) are selected from mixtures of cetyl behenate, stearyl behenate, hydrogenated castor oil, palmitic acid and stearic acid. Further particularly preferred lipid or wax component mixtures a) are selected from mixtures of C20-C40-alkyl stearate, hydrogenated castor oil, palmitic acid and stearic acid. Further preferred deodorant or antiperspirant sticks according to the invention are characterized in that the lipid or wax components a) is present in total in amounts of 4-20% by weight, preferably 8-15% by weight, based on the total composition. In a particularly preferred embodiment, the esters of a saturated, monohydric C50-C60-alcohol and a saturated C8-C36-monocarboxylic acid, which are the lipid or wax components a), are present in amounts of 2-10% by weight, preferably 2-6% by weight, based on the total composition.

Preferred deodorant and antiperspirant preparations according to the invention comprise at least one nonionic oil-in-water emulsifier with an HLB value of more than 7. These are emulsifiers generally known to the person skilled in the art, as are listed for example in Kirk-Othmer, “Encyclopedia of Chemical Technology”, 3rd edition, 1979, volume 8, page 913-916. For ethoxylated products, the HLB value is calculated according to the formula HLB=(100−L):5, where L is the weight fraction of the lipophilic groups, i.e. of the fatty alkyl or fatty acyl groups, in the ethylene oxide adducts, expressed in percent by weight.

Suitable oil-in-water emulsifiers are described in DE 10 2006 021 780 A1, paragraphs [0044] to [0050], preferred amounts in paragraph [0051], to which reference is made at this point in its entirety.

Preferred deodorant and antiperspirant preparations according to the invention further comprise at least one nonionic water-in-oil emulsifier with an HLB value greater than 1.0 and less than/equal to 7.0.

Suitable water-in-oil emulsifiers are described in DE 10 2006 021 780 A1, paragraphs [0053] to [0056], preferred amounts in paragraphs [0057] to [0058], to which reference is made at this point in its entirety.

Preferred deodorant and antiperspirant preparations according to the invention preferably comprise at least one oil. Suitable oils are described in DE 10 2006 021 780 A1, paragraphs [0062] to [0066] and [0073], to which reference is made at this point in its entirety.

Preferred deodorant and antiperspirant preparations according to the invention preferably comprise at least one polyol. Suitable polyols are described in DE 10 2006 021 780 A1, paragraphs [0080] and [0081], to which reference is made at this point in their entirety.

Preferred deodorant and antiperspirant preparations according to the invention further comprise at least one deodorant and/or antiperspirant active ingredient.

Deodorant active ingredients preferred according to the invention are odor absorbers, deodorizing ion exchangers, antimicrobial agents, prebiotically effective components and enzyme inhibitors or, particularly preferably, combinations of said active ingredients. Suitable deodorant active ingredients are described in DE 10 2006 021 780 A1, paragraphs [0087] and [0093], to which reference is made at this point in its entirety. The amount of deodorant active ingredients (one or more compounds) in the preparations is preferably 0.01 to 10% by weight, particularly preferably 0.05 to 5% by weight, in particular 0.1 to 1% by weight, based on the total weight of the preparation.

Preferred deodorant or antiperspirant preparations according to the invention are characterized in that at least one antiperspirant active ingredient, selected from the water-soluble astringent inorganic and organic salts of aluminum, zirconium and zinc or any desired mixtures of these salts, is present. Suitable antiperspirant active ingredients are described in DE 10 2006 021 780 A1, paragraphs [0095] and [0096], to which reference is made at this point in its entirety.

In a further particularly preferred embodiment, the preparations according to the invention can comprise both at least one deodorant active ingredient and also at least one antiperspirant active ingredient.

Preferred deodorant and antiperspirant preparations according to the invention can also comprise low-melting lipid or wax components, as described in DE 10 2006 021 780 A1, paragraph [98], to which reference is made at this point in its entirety.

Preferred deodorant and antiperspirant preparations according to the invention can also comprise fillers, as described in DE 10 2006 021 780 A1, paragraph [100] and [101], to which reference is made at this point in its entirety.

Preferred deodorant and antiperspirant preparations according to the invention comprise fragrances, as described in DE 10 2006 021 780 A1, paragraphs [105] to [108], to which reference is made at this point in its entirety.

Preferred preparations according to the invention in the form of deodorant or antiperspirant sticks are characterized in that pigments, e.g. titanium dioxide, are also present. The pigment content aids the cosmetic acceptance of the preparation by the user. Furthermore, particularly preferred deodorant or antiperspirant sticks according to the invention are characterized in that they comprise the customary constituents of cosmetic preparations, e.g. dyes, nanospheres, preservatives and photoprotective agents, antioxidants, enzymes and care substances. In particularly preferred deodorant or antiperspirant sticks according to the invention, these are preferably present in an amount of from 0.001 to 20% by weight.

The above-described antioxidants, free-radical scavengers, UV filters, complexing agents and preservatives can be used for product stabilization.

In one embodiment of the invention, the deodorant or antiperspirant preparations comprise hair growth inhibitors. Suitable hair growth inhibitors are described in DE 10 2006 021 780 A1, paragraph [0120], to which reference is made at this point in its entirety.

### Hair Colorants

The polyurethanes PU are also particularly suitable as thickeners for preparations comprising peroxide, such as, for example, hair colorants. A further embodiment of the present invention is thus hair colorants comprising the polyurethanes PU and the use of the polyurethanes PU as thickeners in hair colorants. By using PU, in particular drop-free and/or non-thread-drawing systems can be built up.

Compositions for coloring hair (hair colorants) are divided into three classes depending on their color stability: temporary hair colorants, which withstand only 1-2 hair washes, semipermanent hair colorants, which have to be renewed after 8-10 hair washes, and permanent hair colorants, which cannot be washed out.

Temporary and semipermanent hair colorants are referred as nonoxidative. Here, the dyes position themselves on the keratin of the hair or penetrate into the hair fiber. In the case of permanent hair colorants, the most widespread hair colorants by far, the colors are formed directly on and in the hair from colorless precursors by a chemical reaction of hydrogen peroxide, which serves as oxidizing agent. In this case, the hair is completely colored through, the color cannot be washed out. These hair colorants are referred to as oxidative hair colorants.

Permanent hair coloring is very resistant to hair washing, the effect of light and other hair treatment methods. It is the most widespread and has a market share of ca. 80% among the hair colorants. It only needs to be renewed about every month, due to hair growth. In this coloring system, the dyes are formed directly on and in the hair, by chemical reactions to which the undyed intermediate products or precursors are subjected. Here, oxidation reactions and coupling processes or condensations take place, which are brought about by hydrogen peroxide in the presence of ammonia or monoethanolamine. The use of hydrogen peroxide as oxidizing agent is therefore required because it not only initiates the dye formation, but at the same time also destroys the melanin pigments in the hair and in so doing brings about a bleaching, for which reason this coloring process is also described as a lightening coloration. Permanent hair colorants also in principle include the so-called self-oxidizing dyes, which are oxidized merely by atmospheric oxygen.

Hair colorants are usually in the form of aqueous—preferably thickened—solutions or emulsions and, besides dyes, comprise for example fatty alcohols and/or other oil components, emulsifiers and surfactants, and if appropriate alcohols.

Oxidation hair colorants generally consist of two components, namely

(A) the dye carrier mass comprising the dyes and
(B) the oxidizing agent preparation.

These components are mixed shortly before application and are then applied to the fibers to be colored.

Customary application forms for such permanent or oxidation hair colorants are cream hair colors, hair coloring gels and coloring shampoos. In order to ensure that the active ingredients of the hair coloring compositions remain on the hair for a certain time following application and do not reach areas where they are undesired, such as, for example, the face, the compositions must have a certain minimum viscosity. This viscosity is usually achieved through the use of thickeners, which are thus an essential constituent of most oxidation hair colorants.

The thickeners used are usually crosslinked polyacrylic acids (e.g. Carbopol®), hydroxyethylcellulose, waxes and particularly mixtures of nonionic surfactants with a certain HLB value (Hydrophobic Lipophilic Balance), anionic, cationic or nonionic association polymers. On account of the sometimes very high salt concentrations, the widespread thickener systems based on surfactants and also polyacrylic acids are often no longer able to impart the necessary viscosity to the preparations. Thickeners based on polyacrylic acid have, apart from the low salt tolerance, the further disadvantage that the feel to the touch of the treated hair deteriorates. Often, they do not allow, or allow only to an inadequate degree, the hair to be colored easily and evenly, i.e. with the lowest possible selectivity, and at the same time to convert it to a good cosmetic condition. The viscosities of the compositions are often not stable but decrease with time, meaning that the compositions no longer adhere to the hair and run off onto the scalp, where they then cause an undesired coloration.

An object of this invention then consisted in providing thickeners for water-comprising cosmetic compositions for the coloring of keratin fibers which have a sufficiently high viscosity such that the hair colorant remains at the desired point to be colored for the duration of the application. The compositions thickened in this way should be drip-resistant, color the hair easily and evenly, i.e. with the lowest possible selectivity and also convert it to a good cosmetic condition. In particular, the thickening effect for the application desired in each case should be adequate even at high salt (electrolyte) concentrations.

In preferred preparations according to the invention for the coloring of keratin fibers, the polyurethane PU is preferably present initially in the peroxide-comprising component at a pH in the range from 1 to 4, preferably from 2 to 3. After mixing with the component comprising the dye precursors, the polyurethane PU is present in the mixture at a pH in the range from 7 to 12, preferably from 8 to 10, particularly preferably from 8.5 to 9.5 and especially at a pH of about 9.

Cosmetic hair coloring compositions which comprise polyurethane PU can easily be formulated to give gels with very good properties.

Cosmetic preparations according to the invention for the coloring of keratin fibers comprise, based on the composition, preferably 0.05 to 10% by weight, particularly preferably 1 to 7 and in particular 2 to 6% by weight, of at least one polyurethane PU. A preferred subject matter of the invention is a cosmetic composition for the coloring of keratin fibers comprising at least 2 components (A) and (B), where

component (A) comprises at least one oxidation dye and
component (B) comprises at least one oxidizing agent and at least one polyurethane PU.

Advantageously, (A) and (B) are prepared separately from one another and are brought into contact with one another at most 30 seconds, preferably at most 20 seconds, before being brought into contact with the keratin fibers.

The polyurethane PU acting as thickener may be present in component A and/or B. In one preferred embodiment, the polyurethane PU is present in component B.

### Oxidation Dyes

These compounds, which in the starting state are not dyes in the actual sense, but dye precursors, are divided into oxidation bases (developers) and couplers (nuancers) according to their chemical nature.

Oxidation bases are aromatic compounds which are ring-substituted with at least two electron-donating groups (e.g. amino and/or hydroxy groups) and can therefore be oxidized easily.

Important representatives of these bases are p- and o-phenylenediamine, p- and o-aminophenol, and p- and o-dihydroxybenzene and numerous derivatives which are derived from these compounds, e.g. by substituting the amino group by the methoxy group or by replacing the benzene ring with other ring systems such as pyridine, indole, quinoline, etc.

Among the oxidation dyes, however, p-phenylenediamine and p-tolylenediamine assume a dominant position as dye bases.

The couplers are likewise aromatic compounds which, like the oxidation bases, carry readily oxidizable groups (likewise amino and/or hydroxy groups) on the ring, but in the m position. Important couplers are m-phenylenediamine m-aminophenol and m-dihydroxybenzene.

The hair colorant according to the invention preferably comprises oxidation dye precursors, with which the coloration is produced under the action of oxidizing agents, such as, for example, hydrogen peroxide, or in the presence of atmospheric oxygen.

Suitable oxidation dye precursors which may be mentioned are for example the following developer substances and coupler substances and self-coupling compounds:

Suitable developer substances are for example those described in WO 02/00181, p. 8, I.34 to p. 13, I.28 and those described in DE 103 51 842 A1, paragraph [0015], to which reference is hereby made in its entirety.

Suitable coupler substances are for example those described in WO 02/00181, p. 13, I.30 to p. 14, I.14 and those described in DE 103 51 842 A1, paragraph [0016], to which reference is hereby made in its entirety.

The total amount of the oxidation dye precursors present in the compositions according to the invention is about 0.01 to 12 percent by weight, in particular about 0.2 to 6 percent by weight.

To achieve certain color nuances, customary natural and/or synthetic direct dyes, for example so-called plant dyes such as henna or indigo, triphenylmethane dyes, aromatic nitro dyes, azo dyes, quinone dyes, cationic or anionic dyes, may also additionally be present in the compositions.

Suitable synthetic dyes are for example those described in DE 103 51 842 A1, paragraph [0017] to [0019], to which reference is hereby made in its entirety.

If the preparations according to the invention comprise direct dyes, then the amount, based on the preparation, is about 0.01 to 7 percent by weight, preferably about 0.2 to 4 percent by weight.

Further dyes that are customary and known for hair coloring which may be present in the preparations according to the invention are described inter alia in E. Sagarin, “Cosmetics, Science and Technology”, Interscience Publishers Inc., New York (1957), pages 503 ff. and H. Janistyn, “Handbuch der Kosmetika und Riechstoffe [Handbook of Cosmetics and Fragrances]”, volume 3 (1973), pages 388 ff. and K. Schrader “Grundlagen und Rezepturen der Kosmetika [Fundamentals and Formulations of Cosmetics]”, 2nd edition (1989), pages 782-815, to which reference is hereby expressly made.

Although oxidation hair colorants are preferred, it is of course likewise possible that the compositions according to the invention are present in the form of a nonoxidative colorant based on the aforementioned direct dyes.

Preferred preparations according to the invention comprise as components (A) and (B)

(A) at least one developer substance and/or at least one additional coupler substance and/or at least one direct dye and
(B) at least one oxidizing agent and at least one polyurethane PU.

A further subject matter of the invention is a preparation comprising at least one oxidizing agent and at least one polyurethane PU.

Moreover, the preparations according to the invention can comprise antioxidants such as, for example, ascorbic acid, thioglycolic acid or sodium sulfite, and also complexing agents for heavy metals, for example ethylenediaminotetraacetate or nitriloacetic acid, in an amount of up to about 0.5 percent by weight.

Furthermore, the preparations according to the invention can preferably comprise further additives customary for hair colorants, such as, for example, higher fatty alcohols, preservatives, complexing agents, solvents such as lower aliphatic alcohols, for example ethanol, propanol or isopropanol, or glycols, such as glycerol or 1,2-propylene glycol, wetting agents or emulsifiers from the classes of anionic, cationic, amphoteric or nonionogenic surface-active substances, softeners, vaseline, silicone oils, paraffin oil, polysorbates and fatty acids, and furthermore care substances, such as cationic polymers or resins, lanolin derivatives, cholesterol, vitamins, pantothenic acid and betaine. Although not required, the preparations can also comprise further thickeners such as, for example, homopolymers of acrylic acid, hydrophobically modified polyacrylic acid, plant gums, cellulose and starch derivatives, algae polyasaccharides or amphiphilic associative thickeners.

The constituents mentioned are used in the amounts customary for such purposes, for example the wetting agents and emulsifiers in concentrations of from 0.1 to 30 percent by weight and the care substances in a concentration of from 0.1 to 5.0 percent by weight, based on the preparation.

The preparation according to the invention is preferably formulated in the form of an aqueous or aqueous-alcoholic preparation, for example as thickened solution, as emulsion, as cream or as gel.

For the application for the oxidative coloring, the above-described component A is generally mixed directly prior to use with the component B comprising the oxidizing agent, and an amount of the ready-to-use preparation adequate for the coloring, generally about 60 to 200 grams, is applied to the fibers.

In the case of nonoxidative colorants based on direct dyes, the pH of the preparation according to the invention is in the range from about 5 to 10, preferably 6 to 9.

The preparations for the colouring of keratin fibers are two-component or multicomponent systems, i.e. systems which are mixed prior to bringing them into contact with the keratin fiber, thus the pH of this mixture is in the range from 6 to 12, preferably from 8 to 11, particularly preferably from 8.5 to 9.5 and in particular the pH is about 9.

The pH of the peroxide-comprising component is in the range from 1 to 4, preferably from 2 to 3. The pH of the component comprising the dye precursors and coupler (color carrier mass) is in the range from 9 to 11, preferably about 10. The pH of the ready-to-use preparation according to the invention is established upon the mixing of the preferably alkaline-adjusted color carrier mass with the mostly acidically-adjusted oxidizing agent to a pH which is determined by the amounts of alkali in the color carrier mass and the amounts of acid in the oxidizing agent and also by the mixing ratio.

The polyurethane PU suitable for the use according to the invention may be present in one or in both of the aforementioned components. Preferably, it is present in the component comprising the peroxide and is thus also present at a pH in the range from 1 to 4, preferably from 2 to 3.

Depending on the preparation and the desired pH of the preparation, the pH is preferably adjusted using ammonia or organic amines, such as for example glucamines, aminomethylpropanol, monoethanolamine or triethanolamine, inorganic bases, for example sodium hydroxide, potassium hydroxide, sodium carbonate or calcium hydroxide, or organic or inorganic acids, such as for example lactic acid, citric acid, acetic acid or phosphoric acid.

If the preparation according to the invention comprises no oxidation dye precursors or comprises oxidation dye precursors which can be readily oxidized with atmospheric oxygen, it can be applied directly to the keratin fiber without prior mixing with an oxidizing agent. Suitable oxidizing agents for developing the coloration are primarily hydrogen peroxide or its addition compounds onto urea, melamine or sodium borate in the form of a 1- to 12% strength by weight, preferably 1.5- to 6% strength by weight aqueous solution. The mixing ratio of colorant to oxidizing agent is dependent on the concentration of the oxidizing agent and is generally about 5:1 to 1:2, preferably about 1:1, where the content of oxidizing agent in the ready-to-use preparation is preferably about 0.5 to 8% by weight, in particular 1 to 4% by weight. The ready-to-use colorant is left to act on the keratin fiber (for example human hair) at 15 to 50° C. for about 10 to 45 minutes, preferably about 15 to 30 minutes, then the fiber is rinsed with water and dried. If appropriate this rinsing is followed by washing with a shampoo and possibly rinsing with a weak organic acid, such as, for example, tartaric acid. The keratin fiber is then dried.

The invention provides a method for coloring keratin fibers, in particular human hair, wherein the preparation according to the invention is brought into contact with the keratin fibers to be colored and the coloration takes place in the pH range from 8 to 10, where component (A) and component (B) are mixed prior to being brought into contact with the keratin fibers, and the bringing into contact takes place when the mixture has a dynamic viscosity of at least 3000 mPa*s.

### Haircare Agents

It is the aim of haircare to obtain the natural state of freshly regrown hair over a long period and, in the event of its loss, to restore it again as far as possible. Radiant shine and a pleasant, smooth feel are features of natural healthy hair.

Within the context of this invention, the haircare agents are pretreatment agents, hair rinses (hair conditioners, hair balsams), hair treatments, with a distinction being made between the treatment products which remain in the hair (leave-on) and those which are rinsed off (rinse-off), hair tonics, styling agents such as, for example, pomades, styling creams, styling lotions, styling gels (hair gels, wet-look gels, glitter gels), end fluids, hot-oil treatments and foam treatments.

Customary formulations of the specified haircare compositions known to the person skilled in the art are given in “Kosmetik and Hygiene von Kopf bis Fuβ [Cosmetics and Hygiene from head to toe”], ed. W. Umbach, 3rd edition, Wiley-VCH, 2004, chapter 9.2, pp. 247-264, to which reference is made at this point in its entirety. The ingredients present alongside the polyurethanes PU in the haircare compositions have been specified above and below and are in part identical to those which may also be present in the aforementioned shampoos according to the invention. Depending on the field of application, the haircare compositions can be applied as spray, foam, gel, gelspray, cream, lotion or wax.

Hair sprays comprise here both aerosol sprays and also pump sprays without propellant gas. Hair foams comprise both aerosol foams and also pump foams without propellant gas. Hair sprays and hair foams preferably comprise predominantly or exclusively water-soluble or water-dispersible components.

If the compounds used in the hair sprays and hair foams according to the invention are water-dispersible, they can be applied in the form of aqueous microdispersions with particle diameters of usually 1 to 350 nm, preferably 1 to 250 nm. The solids contents of these preparations are here usually in a range from about 0.5 to 20% by weight. These microdispersions generally require no emulsifiers or surfactants for their stabilization.

### Conditioning Agents

Apart from the polyurethanes PU, preferred haircare compositions according to the invention comprise conditioning agents.

Conditioning agents preferred according to the invention are for example which are listed in the International Cosmetic Ingredient Dictionary and Handbook (volume 4, editor: R. C. Pepe, J. A. Wenninger, G. N. McEwen, The Cosmetic, Toiletry, and Fragrance Association, 9th edition, 2002) under section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and also all compounds listed in EP-A 934 956 (pp. 11-13) under “water soluble conditioning agent” and “oil soluble conditioning agent”.

Advantageous conditioning substances are for example the compounds referred to in accordance with INCI as Polyquaternium (in particular Polyquaternium-1 to Polyquaternium-87) and have already been listed above in the form of a table.

The suitable conditioning agents also include for example polymeric quaternary ammonium compounds, cationic cellulose derivatives, starch derivatives, maltodextrin derivatives and polysaccharide derivatives, and also quaternary protein hydrolysates and quaternary silicone derivatives.

Further conditioners advantageous according to the invention are cellulose derivatives, in particular Polyquaternium-10 and Polyquaternium-67 (e.g. Ucare® Polymer Grades, SoftCAT®Polymer Grades (Dow Chemical)) and quaternized guargum derivatives, in particular guar hydroxypropylammonium chloride (e.g. Jaguar Excel®, Jaguar C-14S or C-13S, Jaguar C 162® (Rhodia), CAS 65497-29-2, CAS 39421-75-5).

According to the invention, nonionic poly-N-vinylpyrrolidone/polyvinyl acetate copolymers (e.g. Luviskol®VA 64 (BASF)), anionic acrylate copolymers (e.g. Luviflex®Soft (BASF)), and/or amphoteric amide/acrylate/methacrylate copolymers (e.g. Amphomer® (National Starch)) can also be used advantageously as conditioners.

A preferred subject matter of the present invention is those haircare compositions which are in the form of transparent gels and, based on the total preparation, comprise at least 0.1% by weight, preferably at least 0.2% by weight and particularly preferably at least 0.5% by weight, of electrolytes. The use according to the invention of the polyurethanes PU as thickeners permits the preparation of transparent, stable gels with an electrolyte concentration, based on the total preparation, of at least 0.1% by weight, preferably at least 0.2% by weight, particularly preferably at least 0.5% by weight and at most 10% by weight, preferably 5% by weight, particularly preferably 1% by weight.

Such stable, transparent gels and conditioners cannot be prepared using conventional thickeners.

Preferably, such gels and conditioners comprise further ingredients depending on the field of application. Suitable further ingredients are known to the person skilled in the art and described in detail above.

### Acidic Preparations

A large number of cosmetic preparations comprise active ingredients which develop their desired effect particularly at acidic pH values. These include for example preparations which comprise alpha-hydroxycarboxylic acids (AHA) and beta-hydroxycarboxylic acids (BHA) since these are not effective or not very effective in the neutralized state.

Using conventional thickeners, it is not possible, or possible only with difficulties, to thicken such preparations such that the preparation is stable over a prolonged period.

Accordingly, cosmetic and dermatological preparations which, besides the polyurethane PU, comprise at least one active ingredient which develops its cosmetic and/or dermatological effectiveness at acidic pH values, i.e. in the range from 1 to less than 7, are in accordance with the invention.

Preferred active ingredients of this type are the alpha-hydroxycarboxylic acids and beta-hydroxycarboxylic acids. Preference is given to those preparations which have a pH in the range from 3 to 6, particularly preferably in the range from 4 to 5.5.

To treat aging phenomena of the skin, keratinization disorders, photodamage and acne, superficial and medium-depth peeling methods are used ever more frequently in dermatology. Surface peeling with alpha-hydroxycarboxylic acids, also called fruit acid peeling, is carried out most often. Glycolic acid has proven to be the most important substance in dermatological practice.

alpha-Hydroxycarboxylic acids have both an epidermal and also dermal effect on the skin. They influence the corneozyte cohesion; thus, old or dead cell material is dissolved and the epidermal horny layer is thinned out. The simultaneous increase in cell turnover leads to thickening of the epidermis below the Stratum corneum. A further epidermal effect is the increased glycosaminoglycan synthesis and a better hydration of the skin brought about thereby. An important dermal effect of alpha-hydroxycarboxylic acids discussed is the new formation of elastic and collagenous fibers, hitherto successfully demonstrated primarily for glycolic acid. Glycolic acid and other alpha-hydroxycarboxylic acids are indicated primarily for ichthyoses, hyperkeratoses, acne and vulgar warts. For therapeutic use, low-concentration (5 to 15 percent) and high-concentration (above 50 percent) preparations are differentiated. Low-concentration solutions and gels can be applied carefully by instructed patients themselves, whereas treatment with more highly concentrated preparations should be carried out by the doctor. For this actual fruit acid peeling, in most cases glycolic acid solutions are used, the concentrations of which can increase in the course of the treatment to up to 70 percent.

One embodiment of the invention is peeling preparations comprising at least one polyurethane PU and at least one alpha-hydroxycarboxylic acid.

The alpha-hydroxy acids are preferably selected from the group consisting of lactic acid, glycolic acid, malic acid, tartronic acid, tartaric acid, glucuronic acid, pyruvic acid, 2-hydroxyisobutyric acid, 3-hydroxyisobutyric acid, citric acid, galacturonic acid, mandelic acid, mucic acid, beta-phenyl acetic acid, beta-phenylpyruvic acid, saccharic acid, alpha-hydroxybutyric acid, alpha-hydroxyisobutyric acid, alpha-hydroxyisocaproic acid, alpha-hydroxyisovaleric acid, atrolactic acid, galactanic acid, pantos acid, glyceric acid, isocitric acid, dihydroxymaleic acid, dihydroxytartaric acid, dihydroxyfumaric acid, benzylformic acid, with lactic acid and glycolic acid being particularly preferred. In one embodiment of the invention, a combination of two acids is used. Suitable beta-hydroxycarboxylic acids are for example salicylic acid and D- and L-carnitine.

Furthermore, esters of the alpha-hydroxy acids can be used, the particular effect of which being that they release the alpha-hydroxy acids more slowly in the skin. These esters include in particular the esters commercially available under the tradenames Cosmacol ETL® (di-C14-C15-alkyl tartrate), Cosmacol ECL® (tri-C14-C15-alkyl citrate), Cosmacol ELI® (C12-C13-alkyl lactate), Cosmacol FOI® (C12-C13-alkyl octanoate), Cosmacol EMI® (Di-C12-C13-alkyl malate), Cosmacol ECI® (tri-C12-C13-alkyl citrate), and Cosmacol ETI® (di-C12-C13-alkyl tartrate). The doses of these esters, alone or in a mixture, are in the range from 2 to 15%, preferably 4 to 10%, of the total weight of the preparation.

### Preparations for Oral Hygiene and Dental Care

A further embodiment of the invention is preparations for oral and dental care and cleaning, comprising at least one polyurethane PU. Dental cleaning compositions are on the market in various forms and serve primarily for the cleaning of the tooth surface and the prevention of dental and gum diseases. They usually comprise a combination of polishes, humectants, surfactants, binders, aroma substances and fluoride-containing and antimicrobial active ingredients. Besides dental powders which, on account of their increased abrasivity, play a minor role, dental cleaning compositions are supplied primarily in paste form, cream form and translucent or transparent gel form. In recent years, liquid dental creams and mouthwashes have also increasingly gained in importance.

Oral and dental care compositions and also oral and dental cleaning compositions within the context of the invention are oral powders and dental powders, oral pastes and toothpastes, liquid oral creams and dental creams, and also oral gels and dental gels. Toothpastes and liquid dental cleaning compositions are preferably suitable. In addition, the oral and dental care and cleaning compositions can be present e.g. in the form of toothpastes, liquid dental creams, dental powders or mouthwashes. Preferably, however, they are present as more or less flowable or plastic toothpastes, as are used for cleaning the teeth using a toothbrush.

In one preferred embodiment of the invention, the dental care preparations comprise nanoparticulate calcium salts, in particular hydroxyapatite, fluorapatite and/or calcium fluoride. Such nanoparticulate calcium salts and their preparation are described for example in DE 10 2006 009 780 A1, EP 934 449, EP-B 1023035, EP 1139995 and in particular in WO 03/000588.

As a further ingredient of the preparations, if appropriate water-soluble surfactants and/or water-soluble polymeric protective colloids are present. The at least one water-soluble surfactant and/or the at least one water-soluble polymeric protective colloid is present, based on the preparation, in the range from 0.01 to 15% by weight, preferably from 0.1 to 10% by weight and in particular from 0.5 to 7.5% by weight. Suitable anionic surfactants are described in DE 10 2006 009 780 A1, paragraphs and [0053], suitable zwitterionic surfactants ibid in paragraph [0054], suitable ampholytic surfactants ibid in paragraph [0055], suitable nonionic surfactants ibid in paragraphs [0056] to [0061] and suitable cationic surfactants ibid in paragraphs to [0065], to which reference is hereby made in its entirety.

Suitable water-soluble polymeric protective colloids are described in DE 10 2006 009 780 A1, paragraphs [0066] to [0099], to which reference is hereby made in its entirety.

The preparations according to the invention can also comprise protein components such as, for example, protein hydrolysates. Protein components suitable according to the invention are described in DE 10 2006 009 780 A1, paragraphs [0115] to [0123], to which reference is hereby made in its entirety.

In addition to the aforementioned ingredients, the oral and dental care and cleaning compositions according to the invention can comprise further ingredients of oral cleaning compositions, mouth care compositions, dental cleaning compositions and/or dental care compositions. The preferred further ingredients are antimicrobial substances as preservatives or as antiplaque active ingredients, anticaries active ingredients, substances effective against tartar, polishes, cleaning bodies, humectants, additional consistency regulators, substances which increase the insensitivity of the teeth, wound-healing and antiinflammatory substances, substances for increasing the mineralizing potential, aroma oils, sweeteners, solvents and solubility promoters, pigments, such as e.g. titanium dioxide, dyes, buffer substances, vitamins, mineral salts and bioactive glasses. Active ingredients are also understood as meaning the lactic acid bacteria of Lactobacillus anti-caries, which prevent harmful bacteria from settling on the tooth surface and harming the teeth. Preferred further ingredients are described in DE 10 2006 009 780 A1, paragraphs to [0182], to which reference is hereby made in its entirety.

The preparations for oral and dental care and cleaning comprising polyurethanes PU are characterized in that their viscosity (consistency) can be built up even in the presence of electrolytes and/or pigments and also fluoride and has long-term stability.

### Preparations for Hair Removal

A further embodiment of the invention is preparations for hair removal comprising a polyurethane PU. Preparations for hair removal are provided in particular as depilatory creams, depilatory lotions or depilatory foams. The mode of action is based on using reducing agents in the alkaline range to cleave peptide bonds and disulfide bridges of hair keratin so that the hairs are completely removed. The most important hair removal agents are thioglycolic acid and thiolactic acid. Conventional thickeners only allow the thioglycolic acid, thiolactic acid and other basic hair removal agents to be stably formulated at the required alkaline pH values to an inadequate extent. Thus, although xanthan gum has a thickening effect in this pH range, the products thickened in this way have an undesired, slimy consistency. Preparations which have been thickened with customary acrylate thickeners do not exhibit stable consistency.

In contrast to this, typical preparations for hair removal can be thickened with the help of the polyurethanes PU such that the consistency is stable and cosmetically acceptable.

### Preparations for Permanent Hair Shaping

A further embodiment of the invention is preparations for permanent hair shaping comprising a polyurethane PU. The classic technique for carrying out permanent hair shaping consists in, in a first stage, opening the disulfide bonds of the hair keratin using an agent which comprises a reducing active ingredient (shaping agent), then shaping the hair as desired and then joining the disulfide bonds again using an agent comprising an oxidizing active ingredient (neutralizer).

High electrolyte concentrations, oxidative or reductive media and drastic pH conditions are typical for preparations for permanent hair shaping. Customary thickeners do not lead to the desired stable thickened consistency. This is achieved through the use of the polyurethanes PU.

In particular sulfites, thioglycolic acid, thiolactic acid, 3-mercaptopropionic acid, mercaptocarboxylic acid esters and cysteines are used as reducing active ingredients in the preparations for permanent hair shaping. These compositions are either rendered acidic (sulfite, bisulfite and mercaptocarboxylic acid esters) or alkaline (alkali metal and ammonium salts of mercaptocarboxylic acids). In the case of shaping compositions rendered alkaline, the required alkalinity is achieved primarily by adding ammonia, organic amines, ammonium or alkali metal carbonate and ammonium or alkali metal hydrogencarbonate. The neutralizers used are in particular hydrogen peroxide-containing or bromate-containing liquids.

The preparation according to the invention for permanent hair shaping comprises the keratin-reducing compounds in the amounts customary for hair shaping, for example the ammonium salts of thioglycolic acid or thiolactic acid or else cysteine in a concentration of from 6 to 12 percent by weight. The pH of the alkaline shaping compositions is generally 7 to 10, adjustment preferably taking place with ammonia, monoethanolamine, ammonium carbonate or ammonium hydrogencarbonate.

If the preparation is rendered acidic (for example to pH=6.5 to 6.9), esters of mercaptocarboxylic acids, such as for example monothioglycolic acid glycol esters or glycerol esters, but preferably mrcaptoacetamides or 2-mercaptopropionamides, are used in a concentration of 2 to 14% by weight, based on the preparation, or else the salts of sulfurous acid, for example sodium, ammonium or monoethanolammonium sulfite, in a concentration of 3 to 8% by weight, based on the preparation (calculated as SO₂). Preferably, the hair-keratin-reducing compound used is the salt or the derivative of a mercaptocarboxylic acid. The keratin-reducing compound is particularly preferably selected from thioglycolic acid, cysteine and thiolactic acid or salts thereof.

To increase the effect, swelling and penetration substances, such as for example urea, polyhydric alcohols, ethers, melamine, alkali metal or ammonium thiocyanate, isopropanol, imidazolidin-2-one, 2-pyrrolidone and 1-methyl-2-pyrrolidone, can be added in a concentration of about 0.5 to 50 percent by weight, preferably 2 to 30 percent by weight (based on the preparation).

The preparation advantageously additionally comprises the disulfide of a hair-keratin-reducing compound, in particular dithioglycolate. The preferred use amount is 2 to 20% by weight, preferably 3 to 10% by weight, in each case based on the preparation, with a weight ratio between hair-keratin-reducing compound and the disulfide preferably being from 2:1 to 1:2, in particular 2:1 to 1:1.

The preparations according to the invention can be present in the form of an aqueous solution or an emulsion, and also in thickened form based on water, in particular as cream, gel or paste.

The preparations according to the invention can of course comprise all additives known and customary for such compositions, for example further thickeners, such as, for example, kaolin, bentonite, fatty acids, higher fatty alcohols, starch, polyacrylic acid, cellulose derivatives, alginates, vaseline or paraffin oil, wetting agents or emulsifiers from the classes of anionic, cationic, amphoteric or nonionogenic surface-active substances, for example fatty alcohol sulfates, fatty alcohol ether sulfates, alkylsulfonates, alkylbenzene sulfates, quaternary ammonium salts, alkylbetaines, oxethylated fatty alcohols, oxethylated alkylphenols, fatty acid alkanolamides or oxethylated fatty acid esters, also opacifiers, such as, for example, polyethylene glycol esters, or alcohols, such as, for example, ethanol, propanol, isopropanol or glycerol, solubility promoters, stabilizers, buffer substances, perfume oils, hair conditioning agents, and haircare constituents, such as, for example, cationic polymers, lanolin derivatives, cholesterol, pantothenic acid, creatine or betaine. Suitable cationic polymers are described in DE 10 2004 054 055 A1, paragraphs to [0044], to which reference is made at this point in its entirety. Further suitable cationic-active haircare compounds which may be present in the preparations according to the invention are cationically modified protein derivatives or cationically modified protein hydrolysates and are described in DE 10 2004 054 055 A1, paragraphs [0045] to [0046], to which reference is made at this point in its entirety.

The polyurethanes PU are also characterized in that they permit a reversible lowering of viscosity at elevated temperatures in the preparations according to the invention. Consequently, preparations can be formulated more easily and more quickly following their production at elevated temperatures.

The invention is illustrated in more detail by reference to the following nonlimiting examples.

### EXAMPLES

Unless stated otherwise, all of the percentages are percentages by weight.

### Determination of the Dynamic Viscosity

The dynamic viscosities of the polyurethanes PU used according to the invention in aqueous dispersion were measured in the form of a 10 percent strength by weight dispersion at 23° C. In the examples listed below, the dynamic viscosity was for this purpose always determined at shear rates of 100 1/s and 350 1/s. These two values allow a statement to be made as to whether the polyurethanes PU used according to the invention exhibit structurally-viscous or Newtonian thickening behavior in aqueous dispersion.

The following were used for determining the dynamic viscosity in accordance with DIN53019:
Instrument used: Physica Rheolab MC1 Portable rotary viscometer from Anton Paar;
Cylinder measurement system, Z4 DIN cylinder (diameter 14 mm)
Instrument used: Physica Rheolab MC1 Portable rotary viscometer from Anton Paar;
Cylinder measurement system, Z4 DIN cylinder (diameter 14 mm)

### Synthesis Example 1

### Preparation of Polyurethanes PU.1

17.75 kg of a linear polyethylene glycol with a number-average molecular weight of 6000 g/mol (e.g. Pluriol® E6000 from BASF SE) were dissolved in 23.50 kg of xylene under nitrogen. After heating the solution to ca. 140° C., xylene was distilled off so that the water content of the reaction mixture was then only ca. 140 ppm.

The polymer solution was now cooled to 50° C. and admixed with 13.1 g of acetic acid, dissolved in 500 ml of xylene, in order to buffer the amount of potassium acetate in the polyethylene glycol which had been quantitatively determined beforehand. By adding 37.28 g of zinc neodecanoate, dissolved in a mixture of aliphatic hydrocarbons and xylene, and 870.0 g of hexamethylene diisocyanate, dissolved in xylene, the polymerization was started and the mixture was left to react at 50° C. until the isoyanate content was 0.27% by weight.

A mixture of 1.42 kg of a nonionic ethoxylated fatty alcohol, prepared from a saturated iso-C13 alcohol and an average degree of ethoxylation of 10 (e.g. Lutensol® TO10 from BASF SE), and 1.64 kg of a nonionic ethoxylated fatty alcohol mixture, prepared from a saturated C16/C18 alcohol mixture and an average degree of ethoxylation of 11 (e.g. Lutensol® AT11 from BASF SE), dissolved in xylene, was then added. The reaction mixture was further heated at 50° C. until the isocyanate content was 0% by weight. The solvent xylene was then subsequently removed by vacuum distillation at elevated temperature down to a residual content of below 500 ppm.

The resulting product PU.1 is a mixture which comprises linear polyurethanes with edge-position branched and/or unbranched sections T. The ratio of the molecular weights of a hydrophilic section S to the molecular weight of a hydrophilic section P in the polyurethanes PU.1 is typically 1:12.4 or 1:13.6. The latter ratio arises for sections S which consist of 10 ethylene oxide radicals, and the first for those which are composed of 11 ethylene oxide radicals.

The molar ratio of sections P to D is 1:1.75.

The product PU.1 was dispersed in 86.73 kg of water and cooled to room temperature (25° C.). The mixture of polymers PU.1 (Mn=17 600 g/mol; Mw=30 500 g/mol) was in the form of an aqueous dispersion which had a solids content of 20.5% by weight. The viscosity of a 10 percent strength by weight aqueous dispersion of the polyether polyurethanes PU.1 at 23° C. was 7700 mPa*s (shear rate 100 1/s) or 5900 mPa*s (shear rate 350 1/s) and exhibited weak structurally viscous behavior.

### Synthesis Example 2

### Preparation of Polyurethanes PU.2

17.75 kg of a linear polyethylene glycol with a number-average molecular weight of 6000 g/mol (e.g. Pluriol® E6000 from BASF SE) were dissolved in 23.50 kg of xylene under nitrogen. After heating the solution to ca. 140° C., xylene was distilled off so that the water content of the reaction mixture was then only ca. 250 ppm.

The polymer solution was now cooled to 50° C. and admixed with 13.1 g of acetic acid, dissolved in 500 ml of xylene, in order to buffer the amount of potassium acetate in the polyethylene glycol which had been quantitatively determined beforehand.

By adding 37.28 g of zinc neodecanoate, dissolved in a mixture of aliphatic hydrocarbons and xylene, and 870.0 g of hexamethylene diisocyanate, dissolved in xylene, the polymerization was started and the mixture was left to react at 50° C. until the isocyanate content was 0.29% by weight.

A mixture of 0.95 kg of a nonionic ethoxylated fatty alcohol, prepared from a saturated iso-C13 alcohol and an average degree of ethoxylation of 10 (e.g. Lutensol® TO10 from BASF SE), and 2.19 kg of a nonionic ethoxylated fatty alcohol, prepared from a saturated C16/C18 alcohol mixture and an average degree of ethoxylation of 11 (e.g. Lutensol® AT11 from BASF SE), dissolved in xylene, was then added and the reaction mixture was further heated at 50° C. until the isocyanate content was 0% by weight.

The solvent xylene was subsequently removed by vacuum distillation at elevated temperature down to a residual content of below 500 ppm.

The resulting product PU.2 is a mixture which comprises linear polyurethanes with edge-position branched and/or unbranched sections T. The ratio of the molecular weights of a hydrophilic section S to the molecular weight of a hydrophilic section P in the polyurethanes PU.2 is typically 1:12.4 or 1:13.6. The latter ratio arises for sections S which consist of 10 ethylene oxide radicals, the former for those which are composed of 11 ethylene oxide radicals.

The molar ratio of sections P to D is 1:1.75.

The product PU.2 was dispersed in 87.02 kg of water and cooled to room temperature (25° C.). The polymer mixture PU.2 (Mn=16 700 g/mol; Mw=29 500 g/mol) was in the form of an aqueous dispersion which had a solids content of 20.0% by weight. The viscosity of a 10 percent strength by weight aqueous dispersion of the polyether polyurethanes PU.2 at 23° C. was 26 200 mPa*s (shear rate 100 1/s) or 12 800 mPa*s (shear rate 350 1/s) and exhibited marked structurally viscous behavior.

### Synthesis Example 3

### Preparation of Polyurethanes PU.3

120.00 g of a linear polyethylene glycol with a number-average molecular weight of 6000 g/mol (e.g. Pluriol® E6000 from BASF SE) were dissolved in 467.00 g of xylene under nitrogen. After heating the solution to ca.140° C., xylene was distilled off so that the water content of the reaction mixture was then less than 300 ppm.

The polymer solution was then cooled to 50° C. By adding 42 mg of zinc neodecanoate, dissolved in a mixture of aliphatic hydrocarbons, and 5.88 g of hexamethylene diisocyanate, dissolved in xylene, the polymerization was started and the mixture was left to react at 50° C. until the isocyanate content was 0.25% by weight.

19.20 g of a nonionic ethoxylated fatty alcohol, prepared from a saturated iso-C13 alcohol and an average degree of ethoxylation of 10 (e.g. Lutensol® TO10 from BASF SE), dissolved in xylene, were then added and the reaction mixture was further heated at 50° C. until the isocyanate content was 0% by weight. The solvent xylene was then removed by vacuum distillation at elevated temperature down to a residual content of below 500 ppm.

The resulting product PU.3 is a mixture which comprises linear polyurethanes with edge-position branched sections T. The ratio of the molecular weights of a hydrophilic section S to the molecular weight of a hydrophilic section P in the polyurethanes PU.3 is typically 1:13.6. This ratio arises for sections S which consist of 10 ethylene oxide radicals.

The molar ratio of sections P to D is 1:1.75.

The product PU.3 was dispersed in 580.3 g of water and cooled to room temperature (25° C.). The polymer mixture PU.3 (Mn=27 200 g/mol; Mw=51 900 g/mol) was in the form of an aqueous dispersion which had a solids content of 20.0%. The viscosity of a 10 percent strength by weight aqueous dispersion of the polyether polyurethanes PU.3 at 23° C. was 680 mPa*s (shear rate 100 1/s) or 640 mPa*s (shear rate 350 1/s) and exhibited Newtonian thickening behavior.

### Synthesis Example 4

### Preparation of Polyurethanes PU.4

17.75 kg of a linear polyethylene glycol with a number-average molecular weight of 6000 g/mol (e.g. Pluriol® E6000 from BASF SE) were dissolved in 23.50 kg of xylene under nitrogen. After heating the solution to ca.140° C., xylene was distilled off so that the water content of the reaction mixture was then ca. 120 ppm.

The polymer solution was then cooled to 50° C. and admixed with 13.1 g of acetic acid, dissolved in 500 ml of xylene, in order to buffer the amount of potassium acetate in the polyethylene glycol which had been quantitatively determined beforehand.

By adding 37.28 g of zinc neodecanoate, dissolved in a mixture of aliphatic hydrocarbons and xylene, and 870.0 g of hexamethylene diisocyanate, dissolved in xylene, the polymerization was started and the mixture was left to react at 50° C. until the isocyanate content was 0.26% by weight.

2.84 kg of a nonionic ethoxylated fatty alcohol, prepared from a saturated iso-C13 alcohol and an average degree of ethoxylation of 10 (e.g. Lutensol® TO10 from BASF SE), dissolved in xylene, were then added and the reaction mixture was further heated at 50° C. until the isocyanate content was 0% by weight. The solvent xylene was then removed by vacuum distillation at elevated temperature until the residual content was below 500 ppm.

The resulting product PU.4 is a mixture which comprises linear polyurethanes with edge-position branched sections T. The ratio of the molecular weight of a hydrophilic section S to the molecular weight of a hydrophilic section P in the polyurethanes PU.4 is typically 1:13.6. This ratio arises for sections S which consist of 10 ethylene oxide radicals.

The molar ratio of sections P to D is 1:1.75.

The product PU.4 was dispersed in 85.84 kg of water and cooled to room temperature (25° C.). The polymer mixture PU.4 (Mn=19 200 g/mol; Mw=30 800 g/mol) was in the form of an aqueous dispersion which had a solids content of 18.1%. The viscosity of a 10 percent strength by weight aqueous dispersion of the polyether polyurethanes PU.4 at 23° C. was 600 mPa*s (shear rate 100 1/s) or 570 mPa*s (shear rate 350 1/s) and exhibited Newtonian thickening behavior.

### Synthesis Example 5

### Preparation of Polyurethanes PU.5

240.00 g of a linear polyethylene glycol with a molecular weight of 6000 g/mol (e.g. Pluriol® E6000 from BASF SE) were dissolved in 934.00 g of xylene under nitrogen. After heating the solution to ca.140° C., xylene was distilled off so that the water content of the reaction mixture was then less than 300 ppm.

The polymer solution was then cooled to 50° C. By adding 84 mg of zinc neodecanoate, dissolved in aliphatic hydrocarbons, and 11.76 g of hexamethylene diisocyanate, dissolved in xylene, the polymerization was started and the mixture was left to react at 50° C. until the isocyanate content was 0.22% by weight.

20.70 g of a nonionic ethoxylated fatty alcohol, prepared from a saturated C13 alcohol and an average degree of ethoxylation of 3 (e.g. Lutensol® AO3 from BASF SE), dissolved in xylene, were then added and the reaction mixture was further heated at 50° C. until the isocyanate content was 0% by weight. The solvent xylene was then removed by vacuum distillation at elevated temperature until the residual content was below 500 ppm and the residue was then dispersed in 1089.8 g of water.

The ratio of the molecular weights of a hydrophilic section S to the molecular weight of a hydrophilic section P in the polyurethanes PU.5 is typically 1:45.5. This ratio arises for the sections S which consist of 3 ethylene oxide radicals.

The molar ratio of sections P to D is 1:1.75.

After cooling to room temperature (25° C.), the polymers PU.5 (Mn=21 300 g/mol; Mw=36 300 g/mol) were in the form of an aqueous dispersion which had a solids content of 20.1% by weight. The viscosity of a 10 percent strength by weight aqueous dispersion of the polyether polyurethanes PU.5 at 23° C. was 10 900 mPa*s (shear rate 100 1/s) or 9200 mPa*s (shear rate 350 1/s) and exhibited weak structurally viscous behavior.

### Synthesis Example 6

### Preparation of Polyurethanes PU.6

180.00 g of a linear polyethylene glycol with a molecular weight of 6000 g/mol (e.g. Pluriol® E6000 from BASF SE) were dissolved in 180.00 g of acetone under nitrogen. After heating the solution to reflux (internal temperature ca. 56° C.), a further 1362.4 g of acetone were continuously added and, at the same time, a total of 1362.4 of acetone were distilled off. The water content of the reaction mixture was then only still ca. 240 ppm.

The polymer solution was then cooled to 50° C. By adding 189 mg of zinc neodecanoate, dissolved in aliphatic hydrocarbons, and 8.82 g of hexamethylene diisocyanate, dissolved in acetone, the polymerization was started and the mixture was left to react at 50° C. until the isocyanate content was 0.33% by weight.

15.53 g of a nonionic ethoxylated fatty alcohol, prepared from a saturated C13 alcohol and an average degree of ethoxylation of 3 (e.g. Lutensol® AO3 from BASF SE), dissolved in acetone, were then added and the reaction mixture was further heated at 50° C. until the isocyanate content was 0% by weight. The solvent acetone was then removed by vacuum distillation down to a residual content of below 500 ppm and the residue was dispersed in 817.4 g of water.

The ratio of the molecular weights of a hydrophilic section S to the molecular weight of a hydrophilic section P in the polyurethanes PU.6 is typically 1:45.5. This ratio arises for the sections S which consist of 3 ethylene oxide radicals.

The molar ratio of sections P to D is 1:1.75.

After cooling to room temperature (25° C.), the polymers PU.6 (Mn=24 900 g/mol; Mw=40 000 g/mol) were in the form of an aqueous dispersion which had a solids content of 19.6% by weight. The viscosity of a 10 percent strength by weight aqueous dispersion of the polyether polyurethanes PU.6 at 23° C. was 8800 mPa*s (shear rate 100 1/s) or 7800 mPa*s (shear rate 350 1/s) and exhibited weak structurally viscous, behavior.

### Synthesis Example 7

### Preparation of Polyurethanes PU.7

120.00 g of a linear polyethylene glycol with a number-average molecular weight of 6000 g/mol (e.g. Pluriol® E6000 from BASF SE) were dissolved in 467.00 g of xylene under nitrogen. After heating the solution to ca. 140° C., xylene was distilled off so that the water content of the reaction mixture was then only still ca. 120 ppm.

The polymer solution was then cooled to 50° C. and admixed with 107 mg of acetic acid, dissolved in 5 ml of xylene, in order to buffer the amount of potassium acetate in the polyethylene glycol which had been quantitatively determined beforehand. By adding 252 mg of zinc neodecanoate, dissolved in a mixture of aliphatic hydrocarbons and xylene, and 5.88 g of hexamethylene diisocyanate, dissolved in xylene, the polymerization was started and the mixture was left to react at 50° C. until the isocyanate content was 0.25% by weight.

22.20 g of a nonionic ethoxylated fatty alcohol mixture, prepared from a saturated C16-C18 alcohol mixture and an average degree of ethoxylation of 11 (e.g. Lutensol® AT11 from BASF SE), dissolved in xylene were then added. The reaction mixture was further heated at 50° C. until the isocyanate content was 0% by weight. The solvent xylene was then removed by vacuum distillation at elevated temperature down to a residual content of below 500 ppm.

The resulting product PU.7 is a mixture which comprises linear polyurethanes with edge-position, unbranched sections T. The ratio of the molecular weights of a hydrophilic section S to the molecular weight of a hydrophilic section P in the polyurethanes PU.7 is typically 1:12.4. This ratio arises for the sections S which consist of 11 ethylene oxide radicals.

The molar ratio of sections P to D is 1:1.75.

The product PU.7 was dispersed in 592.3 g of water and cooled to room temperature (25° C.). The mixture of polymers PU.7 (Mn=18 700 g/mol; Mw=30 900 g/mol) was in the form of an aqueous dispersion which had a solids content of 20.4% by weight. The viscosity of a 10 percent strength by weight aqueous dispersion of the polyether polyurethanes PU.7 at 23° C. was 35 500 mPa*s (shear rate 100 1/s) or 14 500 mPa*s (shear rate 350 1/s) and exhibited strong structurally viscous behavior.

### Synthesis Example 8

### Preparation of Polyurethanes PU.8

180.00 g of a linear polyethylene glycol with a number-average molecular weight of 9000 g/mol (e.g. Pluriol® E9000 from BASF SE) were dissolved in 467.00 g of xylene under nitrogen. After heating the solution to ca.140° C., xylene was distilled off so that the water content of the reaction mixture was then only still ca. 70 ppm.

The polymer solution was then cooled to 50° C. and admixed with 208 mg of acetic acid, dissolved in 5 ml of xylene, in order to buffer the amount of potassium acetate in the polyethylene glycol which had been quantitatively determined beforehand. By adding 378 mg of zinc neodecanoate, dissolved in a mixture of aliphatic hydrocarbons and xylene, and 5.88 g of hexamethylene diisocyanate, dissolved in xylene, the polymerization was started and the mixture was allowed to react at 50° C. until the isocyanate content was 0.27% by weight.

10.20 g of a nonionic ethoxylated fatty alcohol, prepared from a saturated iso-C13 alcohol and an average degree of ethoxylation of 3 (e.g. Lutensol® TO3 from BASF SE), dissolved in xylene, were then added. The reaction mixture was further heated at 50° C. until the isocyanate content was 0% by weight. The solvent xylene was then removed by vacuum distillation at elevated temperature until the residual content was below 500 ppm.

The resulting product PU.8 is a mixture which comprises linear polyurethanes with edge-position branched sections T. The ratio of the molecular weights of a hydrophilic section S to the molecular weight of a hydrophilic section P in the polyurethanes PU.8 is typically 1:68.2. This ratio arises for sections S which consist of 3 ethylene oxide radicals.

The molar ratio of sections P to D is 1:1.75.

The product PU.8 was dispersed in 784.3 g of water and cooled to room temperature (25° C.). The mixture of polymers PU.8 (Mn=27 300 g/mol; Mw=46 500 g/mol) was in the form of an aqueous dispersion which had a solids content of 20.2% by weight. The viscosity of a 10 percent strength by weight aqueous dispersion of the polyether polyurethanes PU.8 at 23° C. was 1060 mPa*s (shear rate 100 1/s & shear rate 350 1/s) and exhibited marked Newtonian behavior.

### Synthesis Example 9

### Preparation of Polyurethanes PU.9

180.00 g of a linear polyethylene glycol with a number-average molecular weight 9000 g/mol (e.g. Pluriol® E9000 from BASF SE) were dissolved in 467.00 g of xylene under nitrogen. After heating the solution to ca.140° C., xylene was distilled off so that the water content of the reaction mixture was then only still ca. 70 ppm.

The polymer solution was then cooled to 50° C. and admixed with 208 mg of acetic acid, dissolved in 5 ml of xylene, in order to buffer the amount of potassium acetate in the polyethylene glycol that had been quantitatively determined beforehand. By adding 378 mg of zinc neodecanoate, dissolved in a mixture of aliphatic hydrocarbons and xylene, and 5.88 g of hexamethylene diisocyanate, dissolved in xylene, the polymerization was started and the mixture was left to react at 50° C. until the isocyanate content was 0.28% by weight.

A mixture of 5.10 g of a nonionic ethoxylated fatty alcohol, prepared from a saturated iso-C13 alcohol and an average degree of ethoxylation of 3 (e.g. Lutensol® TO3 from BASF SE), and 11.10 g of a nonionic ethoxylated fatty alcohol mixture, prepared from a saturated C16/C18 alcohol mixture and an average degree of ethoxylation of 11 (e.g. Lutensol® AT11 from BASF SE), dissolved in xylene, was then added. The reaction mixture was further heated at 50° C. until the isocyanate content was 0% by weight. The solvent xylene was then removed by vacuum distillation at elevated temperature down to a residual content of below 500 ppm.

The resulting product PU.9 is a mixture which comprises linear polyurethanes with edge-position branched and/or unbranched sections T. The ratio of the molecular weights of a hydrophilic section S to the molecular weight of a hydrophilic section P in the polyurethanes PU.9 is typically 1:12.4 or 1:68.2. The last-mentioned ratio arises for sections S which consist of 3 ethylene oxide radicals, the former for those which are composed of 11 ethylene oxide radicals.

The molar ratio of sections P to D is 1:1.75.

The product PU.9 was dispersed in 764.0 g of water and cooled to room temperature (25° C.). The mixture of polymers PU.9 (Mn=25 000 g/mol; Mw=45 500 g/mol) was in the form of an aqueous dispersion which had a solids content of 20.8% by weight. The viscosity of a 10 percent strength by weight aqueous dispersion of the polyether polyurethanes PU.9 at 23° C. was 7500 mPa*s (shear rate 100 1/s) or 4500 mPa*s (shear rate 350 1/s) and exhibited strong structurally viscous behavior.

### Synthesis Example 10

### Preparation of Polyurethanes PU.10

120.00 g of a linear polyethylene glycol with a number-average molecular weight of 1500 g/mol (e.g. Pluriol® E1500 from BASF SE) were dissolved in 467.00 g of xylene under nitrogen. After heating the solution to ca.140° C., xylene was distilled off so that the water content of the reaction mixture was then only still ca. 110 ppm.

The polymer solution was then cooled to 50° C. and admixed with 90 mg of acetic acid, dissolved in 5 ml of xylene, in order to buffer the amount of potassium acetate within the polyethylene glycol which had been quantitatively determined beforehand.

By adding 252 mg of zinc neodecanoate, dissolved in a mixture of aliphatic hydrocarbons and xylene, and 15.72 g of hexamethylene diisocyanate, dissolved in xylene, the polymerization was started and the mixture was left to react at 50° C. until the isocyanate content was 0.29% by weight.

17.41 g of a nonionic ethoxylated fatty alcohol, prepared from a saturated iso-C13 alcohol and an average degree of ethoxylation of 10 (e.g. Lutensol® TO10 from BASF SE), dissolved in xylene, were then added. The reaction mixture was further heated at 50° C. until the isocyanate content was 0% by weight. The solvent xylene was subsequently removed by vacuum distillation at elevated temperature down to a residual content of below 500 ppm.

The resulting product PU.10 is a mixture which comprises linear polyurethanes with edge-position branched sections T. The ratio of the molecular weights of a hydrophilic section S to the molecular weight of a hydrophilic section P in the polyurethanes PU.10 is typically 1:13.6. This ratio arises for sections S which consist of 10 ethylene oxide radicals.

The molar ratio of sections P to D is 1:1.17.

The product PU.10 was dispersed in 612.5 g of water and cooled to room temperature (25° C.). The mixture of polymers PU.10 (Mn=18 600 g/mol; Mw=34 900 g/mol) was in the form of an aqueous dispersion which had a solids content of 20.1% by weight. The viscosity of a 10 percent strength by weight aqueous dispersion of the polyether polyurethanes PU.10 at 23° C. was 165 mPa*s (shear rate 100 1/s & shear rate 350 1/s) and exhibited marked Newtonian behavior.

### Synthesis Example 11

### Preparation of Polyurethanes PU.11

90.00 g of a linear polyethylene glycol with a number-average molecular weight of 1500 g/mol (e.g. Pluriol® E1500 from BASF SE) were dissolved in 467.00 g of xylene under nitrogen. After heating the solution to ca.140° C., xylene was distilled off such that the water content of the reaction mixture was than only still ca. 80 ppm.

The polymer solution was then cooled to 50° C. and admixed with 68 mg of acetic acid, dissolved in 5 ml of xylene, in order to buffer the amount of potassium acetate in the polyethylene glycol that had been quantitatively determined beforehand. By adding 189 mg of zinc neodecanoate, dissolved in a mixture of aliphatic hydrocarbons and xylene, and 17.64 g of hexamethylene diisocyanate, dissolved in xylene, the polymerization was started and the mixture was left to react at 50° C. until the isocyanate content was 0.97% by weight.

99.00 g of a nonionic ethoxylated fatty alcohol, prepared from a saturated iso-C13 alcohol and an average degree of ethoxylation of 20 (e.g. Lutensol® TO20 from BASF SE), dissolved in xylene, were then added. The reaction mixture was further heated at 50° C. until the isocyanate content was 0% by weight. The solvent xylene was then removed by vacuum distillation at elevated temperature down to a residual content of below 500 ppm.

The resulting product PU.11 is a mixture, which comprises linear polyurethanes with edge-position branched sections T. The ratio of the molecular weights of a hydrophilic section S to the molecular weight of a hydrophilic section P in the polyurethanes PU.11 is typically 1:1.7. This ratio arises for sections S which consist of 20 ethylene oxide radicals.

The molar ratio of sections P to D is 1:1.75.

The product PU.11 was dispersed in 826.6 g of water and cooled to room temperature (25° C.). The mixture of polymers PU.11 (Mn=4000 g/mol; Mw=9000 g/mol) was in the form of an aqueous dispersion which had a solids content of 20.0% by weight. The viscosity of a 10 percent strength by weight aqueous dispersion of the polyether polyurethanes PU.11 at 23° C. was 150 mPa*s (shear rate 100 1/s & shear rate 350 1/s) and exhibited marked structurally viscous behavior.

### Determination of the Critical Micelle Concentration

The CMC of the polyurethanes used according to the invention in water was determined using the dynamic light scattering method.

For this, a goniometer SP-86 (ALV-Laser Vertriebsgesellschaft mbH, Langen, Germany) was used as a combined DLS/SLS unit. The unit also comprised an ALV 5000 correlator and a He—Ne laser of wavelength 633 nm (both likewise ALV, Langen). The conditions used for the measurement series comprising concentrations of from 0.0001 g/l to 10 g/l were a measurement angle of 90° at a temperature of 23° C. The evaluation was carried out with the help of the program known in the prior art called CONTIN (Constrained Inversion) with intensity distribution (CONTIN likewise from ALV, Langen).

### Comparative Example

A nonionic, hydrophobically modified ethoxylated polyurethane of the prior art prepared from stearyl alcohol, a diisocyanate and a polyethylene glycol (sold by Rohm & Haas as Aculyn® 46) was used in the comparison for determining the CMC. Aculyn® 46 had no measurable CMC. At concentrations of from 0.001 to 10 g/l, relatively large undefined aggregates in the range 100 to 500 nm were always present as main component.

### CMC of the Polyurethanes of the Present Invention:

For the mixtures of polyurethanes PU.1 and also PU.2 prepared in synthesis example 1 and 2, it was found that, at 0.1 g/l, defined micelles with average particle diameters of 30 nm were present. The CMC for both was therefore less than 0.1 g/l. For the polyurethanes PU.4 used according to the invention and prepared in synthesis example 4, it was found that, at a concentration of PU.4 of 1 g/l, micelles with diameters of 17 nm were present, and at a concentration of 0.1 g/l, both micelles of an average size of 15 nm and also a small fraction of undefined aggregates of a size of approximately 200 nm existed alongside one another. Consequently, in this case too, a CMC of <0.1 g/l was present.

### Preparation Example 1

### Preparation of Cosmetic Preparations Using the Polyurethanes PU.1. to PU.5 with a Nonionic Base (P.1.1 to P.1.5)

The cosmetic preparations were prepared by adding the water phase B to the oil phase A and subsequently admixing the resulting O/W emulsion with the preservative (phase C). This gave the nonionic-based preparations P.1.1 to P.1.5. (Tab. 1).

TABLE 1
Composition of the nonionic-based cosmetic preparations P.1.1 to P.1.5.

| Phase | Ingredients | P.1.1 | P.1.2 | P.1.3 | P.1.4 | P.1.5 |
|---|---|---|---|---|---|---|
| Phase A | Ceteareth-6, stearyl alcohol | 2.0 g | 2.0 g | 2.0 g | 2.0 g | 2.0 g |
| | Ceteareth-25 | 2.0 g | 2.0 g | 2.0 g | 2.0 g | 2.0 g |
| | Cetearyl alcohol | 2.5 g | 2.5 g | 2.5 g | 2.5 g | 2.5 g |
| | Paraffin oil | 5.0 g | 5.0 g | 5.0 g | 5.0 g | 5.0 g |
| | Cetearyl ethylhexanoate | 5.0 g | 5.0 g | 5.0 g | 5.0 g | 5.0 g |
| Phase B | PU | PU.1 | PU.2 | PU.3 | PU.4 | PU.5 |
| | | 0.5 g | 0.5 g | 2.0 g | 2.0 g | 0.5 g |
| | 1,2-Propylene glycol | 5.0 g | 5.0 g | 5.0 g | 5.0 g | 5.0 g |
| | Water | 77.5 g | 77.5 g | 76.0 g | 76.0 g | 77.5 g |
| Phase C | Preservative Euxyl ® K300 | 0.5 g | 0.5 g | 0.5 g | 0.5 g | 0.5 g |
| | (Phenoxyethanol, | | | | | |
| | methylparaben, ethylparaben, | | | | | |
| | butylparaben, propylparaben, | | | | | |
| | isobutylparaben), commercially | | | | | |
| | available from Schülke&Mayr | | | | | |

### Preparation Example 2

### Preparation of Cosmetic Preparations Using the Polyurethanes PU.1. to PU.5; Nonionic Base (P.2.1 to P.2.5)

The cosmetic preparations were prepared by adding the water phase B to the oil phase A and subsequently admixing the resulting O/W emulsion with the preservative (phase C). This gave the nonionic-based preparations P.2.1-P.2.5. (Tab. 2).

TABLE 2
Composition of the nonionic-based cosmetic preparations P.2.1-P.2.5.

| Phase | Ingredients | P.2.1 | P.2.2 | P.2.3 | P.2.4 | P.2.5 |
|---|---|---|---|---|---|---|
| Phase A | Glyceryl stearate | 2.0 g | 2.0 g | 2.0 g | 2.0 g | 2.0 g |
| | Stearyl alcohol | 2.0 g | 2.0 g | 2.0 g | 2.0 g | 2.0 g |
| | Cyclopentasiloxane, | 3.0 g | 3.0 g | 3.0 g | 3.0 g | 3.0 g |
| | Cyclohexasiloxane | | | | | |
| | Dicaprylyl ether | 3.0 g | 3.0 g | 3.0 g | 3.0 g | 3.0 g |
| | Dimethicone | 2.0 g | 2.0 g | 2.0 g | 2.0 g | 2.0 g |
| | Aluminum starch octenylsuccinate | 1.0 g | 1.0 g | 1.0 g | 1.0 g | 1.0 g |
| | PEG-40 stearate | 2.0 g | 2.0 g | 2.0 g | 2.0 g | 2.0 g |
| Phase B | PU | PU.1 | PU.2 | PU.3 | PU.4 | PU.5 |
| | | 0.5 g | 0.5 g | 2.0 g | 2.0 g | 0.5 g |
| | Glycerol | 5.0 g | 5.0 g | 5.0 g | 5.0 g | 5.0 g |
| | Water | 79.0 g | 79.0 g | 77.5 g | 77.5 g | 79.0 g |
| Phase C | Preservative Euxyl ® K300 | 0.5 g | 0.5 g | 0.5 g | 0.5 g | 0.5 g |
| | (phenoxyethanol, methylparaben, | | | | | |
| | ethylparaben, butylparaben, | | | | | |
| | propylparaben, isobutylparaben), | | | | | |
| | commercially available from | | | | | |
| | Schülke&Mayr | | | | | |

Determination of the Dynamic Viscosity of Preparations with Auxiliaries

The dynamic viscosity of preparations comprising water which comprise further auxiliaries, e.g. those cosmetic preparations which are disclosed in a nonlimiting manner in the preparation examples, was determined using a Brookfield viscometer (Brookfield), model DV-II+Pro viscometer (model: RVDVII+Pro). The measurement system used was a RV spindle set at a temperature of 20° C. and 20 rpm shear rate.

### Viscosities of the Cosmetic Preparations P.1.1 to P.1.5 (Nonionic-Based) as a Function of the Salt Concentration

TABLE 3
Viscosities of the cosmetic preparations P.1.1 to P.1.5 as a function of the
salt concentration.

| | Salt added subsequently in portions | | | | |
|---|---|---|---|---|---|
| | Dynamic viscosity [Pa * s] | | | | |
| | 0% by | 0.5% by | 2.0% by | 5.0% by | 10.0% by |
| Preparation | wt. NaCl | wt. NaCl | wt. NaCl | wt. NaCl | wt. NaCl |
| P.1.1 | 33.2 | 24.0 | 13.2 | 7.9 | 7.0 |
| P.1.2 | 39.5 | 29.8 | 14.8 | 11.0 | 11.3 |
| P.1.3 | 4.1 | 6.1 | 6.3 | 7.7 | 8.6 |
| P.1.4 | 3.0 | 4.3 | 3.9 | 4.3 | 2.4 |
| P.1.5 | 11.3 | 9.7 | 6.9 | 5.1 | 3.8 |

Total amount of salt incorporated into phase B
Dynamic viscosity [Pa * s]

| | | | | | |
|---|---|---|---|---|---|
| P.1.1 | 33.6 | — | 39.3 | 39.6 | — |

In the case of added salt, the preparations P.1.3 and P.1.4 exhibit increasing and largely stable viscosities. P.1.1, P.1.2 and P.1.5 still exhibit a good thickening effect even in the case of a moderate addition of salt

### Viscosities of the Cosmetic Preparations P.2.1 to P.2.5 (Nonionic-Based) as a Function of the Salt Concentration

TABLE 4
Viscosities of the cosmetic preparations P.2.1 to P.2.5 as a function of the
salt concentration which has been added subsequently in portions

| | Dynamic viscosity [Pa * s] | | | | |
|---|---|---|---|---|---|
| | 0% by | 0.5% by | 2.0% by | 5.0% by | 10.0% by |
| Preparation | wt. NaCl | wt. NaCl | wt. NaCl | wt. NaCl | wt. NaCl |
| P.2.1 | 23.3 | 18.0 | 15.0 | 10.6 | 5.3 |
| P.2.2 | 16.4 | 11.2 | 9.5 | 7.6 | 4.6 |
| P.2.3 | 13.1 | 14.4 | 15.6 | 18.0 | 20.3 |
| P.2.4 | 5.4 | 13.0 | 13.3 | 15.2 | 13.7 |
| P.2.5 | 27.0 | 30.6 | 23.5 | 23.8 | 16.1 |

In the case of added salt, preparation P.2.5 exhibits stable and sometimes even increasing viscosities. This is even more marked for P.2.3 and P.2.4, these exhibit a large increase in the dynamic viscosities in the event of the addition of salt up to 10% by weight. P.2.1 and P.2.2 still have a good thickening effect even in the case of a moderate addition of salt.

Typical preparations according to the invention are described below, but without limiting the invention to these examples.

The percentages are % by wt. unless expressly described in some other way.

Sunscreen cream 1

| | % | Ingredient | INCI |
|---|---|---|---|
| A | 58.7 | Water dem. | Aqua |
| | 0.1 | Edeta ® BD | Disodium EDTA |
| | 1.0 | Butylene glycol | Butylene Glycol |
| | 2.0 | Uvinul ® MS 40 | Benzophenone-4 |
| | 1.0 | TEA | Triethanolamine |
| | 0.5 | Panthenol ® 75 W | Panthenol |
| | 2.4 | Polyurethane PU.1 | |
| B | 5.0 | Neo Heliopan ® OS | Octyl Salicylate |
| | 3.0 | Eusolex ® 9020 | Avobenzone |
| | 5.0 | Neo Heliopan ® HMS | Homosalate |
| | 8.0 | Uvinul ® N 539 T | Octocrylene |
| | 1.0 | Cremophor ® GS 32 | Polyglyceryl-3 Distearate |
| | 1.0 | Cremophor ® A 6 | Ceteareth-6, Stearyl Alcohol |
| | 1.0 | Cremophor ® A 25 | Ceteareth-25 |
| | 2.0 | Lanette ® E | Sodium Cetearyl Sulfate |
| | 0.5 | Span ® 60 | Sorbitan Stearate |
| | 3.0 | Luvitol ® Lite | Hydrogenated Polyisobutene |
| | 2.0 | Lanette ® O | Cetearyl Alcohol |
| | 1.5 | Lanette ® 16 | Cetyl Alcohol |
| | 1.0 | Cetiol ® SB 45 | Butyrospermum Parkii (Shea Butter) |
| | 0.1 | Vitamin E acetate | Tocopheryl Acetate |
| | 0.2 | Bisabolol rac. | Bisabolol |
| C | 0.5 | Glydant ® LTD | DMDM Hydantoin |

### Preparation

Heat phases A and B separately to ca. 80° C.
Stir phase B into phase A and briefly homogenize.
Cool to ca. 40° C. with stirring, add phase C, cool to room temperature with stirring and briefly homogenize again.

### Viscosities

a) without polyurethane PU.1: 7.2 Pa s (Brookfield RVD VII+/spindle No. 6)
b) with polyurethane PU.1: 56.2 Pa s (Brookfield RVD VII+/spindle No. 7)

Instead of the sunscreen cream comprising the polyurethane PU.1, sunscreen creams comprising one or more of the polyurethanes PU.2 to PU.11 are also prepared.

Sunscreen cream 2

| | % | Ingredient | INCI |
|---|---|---|---|
| A | 2.0 | Cremophor ® A 6 | Ceteareth-6, Stearyl Alcohol |
| | 2.0 | Cremophor ® A 25 | Ceteareth-25 |
| | 5.0 | Luvitol ® EHO | Cetearyl Ethylhexanoate |
| | 5.0 | Paraffin oil, | Mineral Oil |
| | | thick-liquid | |
| | 2.5 | Lanette ® O | Cetearyl Alcohol |
| B | 5.0 | Z-Cote ® MAX | Zinc Oxide, Dimethoxydiphenylsilane/ |
| | | | Triethoxycaprylylsilane Crosspolymer |
| C | 2.4 | Polyurethane PU.1 | |
| | 5.0 | 1,2-Propylene | Propylene Glycol |
| | | glycol | |
| | 70.5 | Water, demin. | Water |
| D | 0.5 | Euxyl ® K 300 | Phenoxyethanol, Methylparaben, |
| | | | Ethylparaben, Butylparaben, |
| | | | Propylparaben |

Preparation
Heat phase A to 80° C., add phase B to phase A.
Homogenize phase A+B for 3 min.
Heat phase C to 80° C., stir into phase A+B and homogenize.
Cool emulsion to 40° C. with stirring.
Add phase D, cool to RT with stirring and homogenize.

### Viscosities

a) without polyurethane PU.1: 1.5 Pa s (Brookfield RVD VII+/spindle No. 6)
b) with polyurethane PU.1: 24.3 Pa s (Brookfield RVD VII+/spindle No. 6)

Instead of the sunscreen cream comprising the polyurethane PU.1, sunscreen creams comprising one or more of the polyurethanes PU.2 to PU.11 are also prepared.

Day cream with UV protection

| | | |
|---|---|---|
| % | ingredient | INCI |

A

| | | |
|---|---|---|
| 3.00 | Tego Care ® 450 | Polyglyceryl-3 Methyl Glucose |
| | | Distearate |
| 3.00 | Lanette ® 18 | Stearyl Alcohol |
| 2.00 | Cutina ® GMS | Glyceryl Stearate |
| 4.00 | Estol ® 1540 | Ethylhexyl Cocoate |
| 5.00 | Luvito ® l EHO | Cetearyl Ethylhexanoate |
| 8.00 | Uvinul ® A Plus B | Ethylhexyl Methoxycinnamate, |
| | | Diethyl-amino Hydroxybenzoyl |
| | | Hexyl Benzoate |

B

| | | |
|---|---|---|
| 5.00 | D-Panthenol 50 P | Panthenol, Propylene Glycol |
| 0.10 | Edeta BD | Disodium EDTA |
| 1.0-5.0 | Polyurethane PU.1 | |
| ad 100 | Water dem. | Aqua dem. |

C

| | | |
|---|---|---|
| 0.20 | Bisabolol nat. | Bisabolol |
| q.s. | Perfume oil | |
| 0.50 | Aloe Vera gel concentrate | Water, Aloe Barbadensis |
| | 10/1 | Leaf Juice |
| 0.50 | Euxyl ® K 300 | Phenoxyethanol, Methylparaben, |
| | | Butylparaben, Ethylparaben, |
| | | Propylparaben, Isobutylparaben |

### Preparation

Heat phases A and B separately to ca. 80° C.
Stir phase B into phase A and briefly homogenize.
Cool to ca. 40° C. with stirring, add phase C, cool to room temperature with stirring and briefly homogenize again.

Instead of the daycream comprising the polyurethane PU.1, daycreams comprising one or more of the polyurethanes PU.2 to PU.11 are also prepared.

Make-up

| | | |
|---|---|---|
| % | ingredient | INCI |

A

| | | |
|---|---|---|
| 4.00 | Dracorin ® 100 SE | Glyceryl Stearate, PEG-100 Stearate |
| 1.00 | Uvinul ® A Plus | Diethylamino Hydroxybenzoyl Hexyl |
| | | Benzoate |
| 3.00 | Uvinul ® MC 80 | Ethylhexyl Methoxycinnamate |
| 0.50 | Emulmetik ® 100 | Lecithin |
| 0.50 | Rylo ® PG 11 | Polyglyceryl Dimer Soyate |

B

| | | |
|---|---|---|
| 0.35 | Sicovit ® Brown 75 E 172 | Iron Oxides |
| 2.00 | Sicovit ® Red 30 E 172 | Iron Oxides |
| 1.00 | Sicovit ® Yellow 10 E 172 | Iron Oxides |
| 2.25 | Prisorine ® 3630 | Trimethylolpropane Triisostearate |

C

| | | |
|---|---|---|
| 5.50 | Dow Corning ® 345 Fluid | Cyclopentasiloxane, Cyclohexasiloxane |
| 4.00 | Tegosoft ® OP | Ethylhexyl Palmitate |
| 1.50 | Jojoba oil | Simmondsia Chinensis (Jojoba) Seed Oil |
| 2.00 | Miglyol ® 840 | Propylene Glycol Dicaprylate/Dicaprate |
| 1.50 | Almond oil, sweet | Sweet Almond (Prunus Amygdalus Dulcis) Oil |
| 0.50 | Vitamin E acetate | Tocopheryl Acetate |
| 1.00 | Cetiol ® SB 45 | Butyrospermum Parkii (Shea Butter) |
| 5.00 | Uvinul ® TiO2 | Titanium Dioxide, Trimethoxycaprylylsilane |
| 0.50 | Dehymuls ® PGPH | Polyglyceryl-2 Dipolyhydroxystearate |

D

| | | |
|---|---|---|
| 5.00 | 1,2-Propylene glycol Care | Propylene Glycol |
| 0.50 | Lutrol ® F 68 | Poloxamer 188 |
| 0.10 | Edeta BD | Disodium EDTA |
| 1.0-5.0 | Polyurethane PU.1 | |
| ad 100 | Water dem. | Aqua dem. |

E

| | | |
|---|---|---|
| 1.00 | Euxyl ® K 300 | Phenonip |
| | | Phenoxyethanol, Methylparaben, |
| | | Ethylparaben, Butylparaben, Propylparaben, |
| | | Isobutylparaben |
| 0.20 | Bisabolol rac. | Bisabolol |
| q.s. | Perfume oil | |

### Preparation

Heat phases A, B, C and D to 70° C. separately from one another.
Homogenize phase B using a triple-roll mill. Stir phase B into phase A.
Briefly homogenize everything again.
Dissolve phase C and stir into phase A+B.
Dissolve phase D, stir into the combined phases A+B+C and homogenize.
Cool to ca. 40° C. with stirring, add phase E and cool to room temperature. Briefly homogenize.

Instead of the make-up comprising the polyurethane PU.1, make-ups comprising one or more of the polyurethanes PU.2 to PU.11 are also prepared.

Tinted daycream

| | | |
|---|---|---|
| % | Ingredient | INCI |

A

| | | |
|---|---|---|
| ad 100 | Water dem. | Aqua dem. |
| 5.00 | Glycerol 87% | Glycerin |
| 4.00 | D-Panthenol 50 P | Panthenol, Propylene Glycol |
| 0.75 | Cloisonne ® Gold | Mica, Titanium Dioxide, Iron Oxides |
| 0.25 | Cloisonne ® Super | Mica, Iron Oxides |
| | Rouge | |
| 1.0-5.0 | Polyurethane PU.1 | |

B

| | | |
|---|---|---|
| 3.00 | Uvinul ® A Plus | Diethylamino Hydroxybenzoyl Hexyl |
| | | Benzoate |
| 7.00 | Luvitol ® Lite | Hydrogenated Polyisobutene |
| 1.50 | Lanette ® O | Cetearyl Alcohol |
| 1.50 | Cutina ® GMS | Glyceryl Stearate |
| 3.50 | Cetiol ® SB 45 | Butyrospermum. Parkii (Shea Butter) |
| 3.50 | Olive oil | Olive (Olea Europaea) Oil |
| 1.00 | Eumulgin ® B 2 | Ceteareth-20 |
| 1.00 | Cremophor ® A6 | Ceteareth-6, Stearyl Alcohol |
| 1.00 | Cetiol ® OE | Dicaprylyl Ether |
| 0.05 | BHT | BHT |

C

| | | |
|---|---|---|
| 0.20 | Sodium Ascorbyl | Sodium Ascorbyl Phosphate |
| | Phosphate | |
| 5.00 | Water dem. | Aqua dem. |

D

| | | |
|---|---|---|
| 1.00 | Euxyl ® PE 9010 | Phenoxyethanol, Ethylhexylglycerin |
| 0.25 | Bisabolol rac. | Bisabolol |
| 1.00 | Vitamin E acetate | Tocopheryl Acetate |
| q.s. | Perfume oil | |

### Preparation

Heat phase A to 80° C.
Heat phase B to ca. 80° C. and stir into phase A with stirring. Homogenize.
Cool to ca. 40° C. with stirring, add phase C D and cool to room temperature with stirring.
Briefly homogenize.

Instead of the tinted daycream comprising the polyurethane PU.1, tinted daycreams comprising one or more of the polyurethanes PU.2 to PU.11 are also prepared.

Deodorant lotion

| | | |
|---|---|---|
| % | Ingredient | INCI |

A

| | | |
|---|---|---|
| 1.50 | Cremophor ® A 6 | Ceteareth-6, Stearyl Alcohol |
| 1.50 | Cremophor ® A 25 | Ceteareth-25 |
| 2.00 | Cremophor ® CO 40 | PEG-40 Hydrogenated Castor Oil |
| 2.00 | Cutina ® GMS | Glyceryl Stearate |
| 2.00 | Lanette ® O | Cetyl Alcohol |
| 2.00 | Softisan ® 100 | Hydrogenated Coco-Glycerides |
| 8.00 | Cetiol ® V | Decyl Oleate |
| 0.50 | Abil ® B 8843 | PEG-14 Dimethicone |
| 0.30 | Farnesol | Farnesol |

B

| | | |
|---|---|---|
| q.s. | Preservative | Preservative |
| 1.0-5.0 | Polyurethane PU.1 | |
| ad 100 | Water dem. | Aqua dem. |

C

| | | |
|---|---|---|
| q.s. | Perfume oil | Fragrance |

D

| | | |
|---|---|---|
| 5-20 | Locron ® L | Aluminum Chlorohydrate |

### Preparation

Heat phases A and B separately to ca. 80° C.
Stir phase B into phase A with homogenization, briefly after-homogenize.
Cool to ca. 40° C., add phases C and D with homogenization and allow to cool to room temperature with stirring.

Instead of the deodorant lotion comprising the polyurethane PU.1, deodorant lotions comprising one or more of polyurethanes PU.2 to PU.11 are also prepared

Hair wax with pigments

| % | Phase | Ingredient | INCI |
|---|---|---|---|
| 5 | A | Cremophor ® | Hydrogenated Castor Oil |
| | | CO 40PEG-40 | |
| 15 | | Cremophor ® A 25 | Ceteareth-25 |
| 0.5-10 | | Polyurethane PU.1 | |
| 15 | | Luvitol ® Lite | Hydrogenated Polyisobutene |
| 3 | | Marlipal ® MG | Laureth-7 |
| 2 | | Brij ® 98 | Oleth-20 |
| 1 | | Euxyl ® PE 9010 | Phenoxyethanol and |
| | | | Ethylhexylglycerin |
| 5 | B | Abil ® B 88183 | PEG/PPG-20/6 Dimethicone |
| ad 100 | | Water dem. | Water dem |
| 1 | | Gemtone ® Emerald | Mica and Titanium Dioxide and |
| | | | Chromium Oxide Greens and |
| | | | Ferric Ferrocyanide |

### Preparation:

I: Separate weighing-in of phases A and B and heating with stirring to 80° C.
II: Combining phase A and B at 80° C. with stirring
III: Cooling to RT with stirring

Instead of the hair wax comprising the polyurethane PU.1, hair waxes comprising one or more of the polyurethanes PU.2 to PU.11 are also prepared.

Hair gel with UV protection

| Phase | % | INCI | Ingredient |
|---|---|---|---|
| A | 44.55 | Aqua dem. | |
| | 0.45 | Acrylates/C10-30 | Alkyl Acrylate Crosspolymer |
| B | 0.36 | Aminomethyl Propanol | |
| C | 0.66 | Panthenol | D-Panthenol 75W ® |
| | 10.00 | PVP/VA Copolymer | Luviskol ® VA 64 W |
| | 2.50 | Polyquaternium-46 | Luviquat ® Hold |
| | 5.00 | Sorbitol | |
| | 0.10 | Disodium EDTA | |
| | 0.5 | Benzophenone-4 | Uvinul ® MS 40 |
| | q.s. | Perfume | |
| | q.s. | PEG-40 Hydrogenated | Cremophor ® CO 40 |
| | | Castor Oil | |
| | q.s. | Preservative | |
| | 5.00 | Alcohol | |
| | ad 55 | Aqua dem. | |
| | 0.5-4 | Polyurethane PU.1 | |

### Preparation:

I: Separate weighing-in of phases A,B and C and, if appropriate, stirring to homogeneity at RT
II: Combining phase B and A at RT with stirring and stirring until homogeneous, then adding phase C with stirring and stirring until smooth.

Instead of the hair gel comprising the polyurethane PU.1, hair gels comprising one or more of the polyurethanes PU.2 to PU.11 are also prepared.

Hair foam

| % | | Ingredient | INCI |
|---|---|---|---|
| qs | Phase A | Deionized water | (Aqua dem.) |
| 11.00 | | Luviquat ® Hold | (Polyquaternium 46) |
| 1.50 | | Uvinul ® MS 40 | (Benzophenone-4) |
| | | | 20% sol., neutr. m. |
| | | | Triethanolamine |
| 0.1-3 | | Polyurethane PU.1 | |
| 0.40 | | D,L Panthenol 50W | (Panthenol) |
| 0.20 | | Masil ® SF 19 CG | (PEG-8 Methicone) |
| 0.40 | | Glydant ® Plus liquid | (DMDM Hydantoin (and) |
| | | | Iodopropynyl |
| | | | Butylcarbamate) |
| 0.20 | Phase B | Cremophor ® CO 40 | (PEG-40 Hydrogenated |
| | | | Castor Oil) |
| 0.40 | | Vitamin E Acetate | (Tocopheryl Acetate) |
| 0.20 | | Bell ® 6101232 | (Fragrance |
| 0.70 | | Rhodasurf ® L-4 | (Laureth-4) |
| 6.00 | Phase C | Propellant gas A46 | (Propane/Isobutane) |

### Preparation:

1. Weighing-in of the substance of phase A with stirring until completely dissolved in the order listed
2. Weighing-in of the substance of phase B with stirring and heating at 40-45° C.
3. Combining phases A and B and transferring to suitable propellant-gas containers for hair foams
4. Closing and filling with propellant gas of phase C.

Instead of the hair foam comprising the polyurethane PU.1, hair foams comprising one or more of the polyurethanes PU.2 to PU.11 are also prepared.

Face cream with 3% Sodium Ascorbyl Phosphate

| | | |
|---|---|---|
| % | Ingredient | INCI |

A

| | | |
|---|---|---|
| 2.00 | Cremophor ® A 6 | Ceteareth-6, Stearyl Alcohol |
| 2.00 | Cremophor ® A 25 | Ceteareth-25 |
| 3.00 | Jojoba oil | Simmondsia Chinensis (Jojoba) |
| | | Seed Oil |
| 3.00 | Lanette ® O | Cetearyl Alcohol |
| 10.00 | Paraffin oil, thick-liquid | Mineral Oil |
| 5.00 | Vaseline | Petrolatum |
| 4.00 | Miglyol ® 812 | Caprylic/Capric Triglyceride |

B

| | | |
|---|---|---|
| 5.00 | 1,2-Propylene glycol Care | Propylene Glycol |
| 0.10 | Edeta BD | Disodium EDTA |
| 1.0-5.0 | Polyurethane PU.1 | |
| 0.30 | Abiol ® | Imidazolidinyl Urea |
| ad 100 | Water dem. | Aqua dem. |

C

| | | |
|---|---|---|
| 0.08 | Sodium hydroxide | Sodium Hydroxide |

D

| | | |
|---|---|---|
| 0.50 | Vitamin E acetate | Tocopheryl Acetate |
| 0.20 | Phenoxyethanol | Phenoxyethanol |
| 3.00 | Sodium Ascorbyl Phosphate | Sodium Ascorbyl Phosphate |

### Preparation

Heat phases A and B separately to ca. 80° C.
Stir phase B into phase A and homogenize.
Stir phase C into phase A+B and homogenize.
Cool to ca. 40° C. with stirring.
Stir in phase C and briefly after-homogenize.
Cool to room temperature with stirring.

Instead of the face cream comprising the polyurethane PU.1, face creams comprising one or more of the polyurethanes PU.2 to PU.11 are also prepared.

Reducing the average droplet size distribution/O/W emulsion

| | | |
|---|---|---|
| % | Ingredient | INCI |

Phase A

| | | |
|---|---|---|
| 2.0 | Lanette ® O | Cetearyl Alcohol |
| 5.0 | Finsolv ® TN | C12-15 Alkyl Benzoate |
| 10.0 | Miglyol ® 812 | Caprylic/Capric Triglyceride |
| 5.0 | Cetiol ® B | Dibutyl Adipate |
| 2.0 | Amphisol ® K | Potassium Cetyl Phosphate |
| 0.5 | Elfacos ® ST-9 | PEG-45/Dodecyl Glycol Copolymer |

Phase B

| | | |
|---|---|---|
| 5.0 | 1,2-Propylene glycol | Propylene Glycol |
| | Care | |
| ad 100 | Water, demin. | Water |
| 1.0-5.0 | Polyurethane PU.1 | |

Phase C

| | | |
|---|---|---|
| 0.5 | Euxyl ® K 300 | Phenoxyethanol, Methylparaben, |
| | | Ethylparaben, Butylparaben, |
| | | Propylparaben |

Heat phases A and B to ca. 80° C. Stir phase B into phase A, homogenize. Cold-stir, stir in phase C, briefly after-homogenize.

Instead of the O/W emulsion comprising the polyurethane PU.1, O/W emulsions comprising one or more of the polyurethanes PU.2 to PU.11 are also prepared.

AHA Cream

| | | |
|---|---|---|
| % | Ingredient | INCI |

A

| | | |
|---|---|---|
| 2.00 | Cremophor ® A 6 | Ceteareth-6, Stearyl Alcohol |
| 2.00 | Cremophor ® A 25 | Ceteareth-25 |
| 8.00 | Paraffin oil, thick-liquid | Mineral Oil |
| 7.00 | Luvitol ® EHO | Cetearyl Ethylhexanoate |
| 6.00 | Cutina ® GMS | Glyceryl Stearate |
| 1.00 | Lanette ® 16 | Cetyl Alcohol |
| 0.20 | Abil ® 350 | Dimethicone |
| 0.20 | Bisabolol nat. | Bisabolol |

B

| | | |
|---|---|---|
| 1.00 | D-Panthenol USP | Panthenol |
| 3.00 | 1,2-Propylene glycol care | Propylene Glycol |
| 1.0-5.0 | Polyurethane PU.1 | |
| 5.00 | Hydroxy acid | |
| q.s | Sodium hydroxide | Sodium Hydroxide |
| q.s. | Preservative | Preservative |
| ad 100 | Water dem. | Aqua dem. |

C

| | | |
|---|---|---|
| q.s. | Perfume oil | Fragrance |

Note
Alpha-hydroxy acids: lactic acid, citric acid, malic acid, glycolic acid
Dihydroxy acid: tartaric acid
Beta-hydroxy acid: salicylic acid

### Preparation

Heat phases A and B separately to ca. 80° C. If appropriate, adjust pH of phase B to 3 using NaOH.
Stir phase B into phase A with homogenization, briefly after-homogenize.
Cool to ca. 40° C., add phase C, after-homogenize again.

Instead of the AHA cream comprising the polyurethane PU.1, AHA creams comprising one or more of the polyurethanes PU.2 to PU.11 are also prepared.

Cream with vitamin A acid

| | | |
|---|---|---|
| % | Ingredient | INCI |

A

| | | |
|---|---|---|
| 1.50 | Cremophor ® A 25 | Ceteareth-25 |
| 1.50 | Cremophor ® A 6 | Ceteareth-6, Stearyl Alcohol |
| 3.00 | Tegin ® | Glyceryl Stearate SE |
| 2.00 | Lanette ® O | Cetearyl Alcohol |
| 10.00 | Luvitol ® EHO | Cetearyl Ethylhexanoate |
| 5.00 | Paraffin oil, thick-liquid | Mineral Oil |
| 0.10 | D,L-Alpha-Tocopherol | Tocopherol |
| 0.10 | Vitamin A acid | Tretionin |

B

| | | |
|---|---|---|
| 1.0-5.0 | Polyurethane PU.1 | |
| 4.00 | 1,2-Propylene glycol Care | Propylene Glycol |
| 0.10 | Edeta BD | Disodium EDTA |
| q.s. | Preservative | Preservative |
| ad 100 | Water dem. | Aqua dem. |

C

| | | |
|---|---|---|
| 0.40 | Triethanolamine care | Triethanolamine |
| 3.00 | Vitamin E acetate | Tocopheryl Acetate |
| 0.10 | Vitamin A acid | Tretionin |
| q.s. | Perfum oil | Fragrance |

### Preparation

Heat phase A and phase B separately to ca. 75° C. Stir phase B into phase
A and homogenize. Cold-stir. Add phase C at ca. 30° C.
Note: The formulation is prepared without protective gas. Bottling must take place in oxygen-impermeable packagings, e.g. aluminum tubes.

Instead of the cream with vitamin A acid comprising the polyurethane PU.1, creams with vitamin A acid comprising one or more of the polyurethanes PU.2 to PU.11 are also prepared.

Hair removal cream 1

| | | |
|---|---|---|
| % | Ingredient | INCI |

A

| | | |
|---|---|---|
| 4.20 | Lanette ® 16 | Cetyl Alcohol |
| 1.26 | Brij ® 35 | Laureth-23 |

B

| | | |
|---|---|---|
| 15.00 | Luviquat ® Care | Polyquaternium-44 |
| 0.90 | D-Panthenol USP | Panthenol |
| 0.35 | Allantoin | Allantoin |
| q.s. | Preservative | Preservative |
| 22.40 | Calcium carbonate | Calcium Carbonate |
| 10.00 | Calcium hydroxide | Calcium Hydroxide |
| 5.40 | Calcium thioglycolate | Calcium Thioglycolate |
| ad 100 | Water dem. | Aqua dem. |
| 1.0-5.0 | Polyurethane PU.1 | |

C

| | | |
|---|---|---|
| q.s. | Perfume oil | Fragrance |

### Preparation

Heat phase A and B separately to ca. 80° C.
Stir phase B into phase A with homogenization, briefly after-homogenize.
Cool to ca. 40° C., add phase C, homogenize again.

Instead of the hair removal cream comprising the polyurethane PU.1, hair removal creams comprising one or more of the polyurethanes PU.2 to PU.11 are also prepared.

Hair removal cream 2

| | | |
|---|---|---|
| % | Ingredient | INCI |

A

| | | |
|---|---|---|
| 1.00 | Cremophor ® A 6 | Ceteareth-6, Stearyl Alcohol |
| 1.00 | Cremophor ® A 25 | Ceteareth-25 |
| 4.00 | Lanette ® O | Cetearyl Alcohol |
| 6.00 | Paraffin oil, thick-liquid | Mineral Oil |
| q.s. | Preservative | Preservative |

B

| | | |
|---|---|---|
| 8.00 | Calcium thioglycolate | Calcium Thioglycolate |
| 2.00 | 1,2-Propylene glycol Care | Propylene Glycol |
| 1.0-5.0 | Polyurethane PU.1 | |
| 1.00 | Sodium hydroxide | Sodium Hydroxide |
| ad 100 | Water dem. | Aqua dem. |

C

| | | |
|---|---|---|
| q.s. | Perfume oil | Fragrance |

### Preparation

Heat phase A and B separately to ca. 80° C.
Stir phase B into phase A with homogenization, briefly after-homogenize.
Cool to ca. 40° C., add phase C, homogenize again.

Instead of the hair removal cream comprising the polyurethane PU.1, hair removal creams comprising one or more of the polyurethanes PU.2 to PU.11 are also prepared.

Conditioner shampoo 1

| | | |
|---|---|---|
| 35.70 | g | Sodium Laureth Sulfate |
| 6.50 | g | Cocamidopropyl Betaine |
| 0.20 | g | Polyurethane PU.1 |
| 0.50 | g | Polyquaternium-7, PQ-10, PQ-39, PQ-44, PQ-67, |
| | | Guarhydroxypropyltrimonium Chloride and/or PQ-87 |
| 0.10 | g | Preservative |
| 0.10 | g | Perfume oil/essential oil |
| ad 100 | g | Aqua dem. |

Instead of conditioner shampoo 1 comprising the polyurethane PU.1, conditioner shampoos comprising one or more of the polyurethanes PU.2 to PU.11 are also prepared.

Conditioner shampoo 2

| | | |
|---|---|---|
| 35.70 | g | Sodium Laureth Sulfate |
| 6.50 | g | Cocamidopropyl Betaine |
| 0.50 | g | Polyurethane PU.1 |
| 0.20 | g | Guarhydroxypropyltrimonium Chloride |
| 0.10 | g | Preservative |
| 0.10 | g | Perfume oil/essential oil |
| ad 100 | g | Aqua dem. |

Instead of the conditioner shampoo 2 comprising the polyurethane PU.1, conditioner shampoos comprising one or more of the polyurethanes PU.2 to PU.11 are also prepared.

### Example 3

Conditioner shampoo 3

| | | |
|---|---|---|
| 35.70 | g | Sodium Laureth Sulfate |
| 6.50 | g | Cocamidopropyl Betaine |
| 0.20 | g | Polyurethane PU.1 |
| 0.50 | g | Polyquaternium-7, PQ-10, PQ-39, PQ-44, PQ-67, |
| | | Guarhydroxypropyltrimonium Chloride and/or PQ-87 |
| 0.10 | g | Preservative |
| 0.10 | g | Perfume oil/essential oil |
| ad 100 | g | Aqua dem. |

Instead of conditioner shampoo 4 comprising the polyurethane PU.1, conditioner shampoos comprising one or more of the polyurethanes PU.2 to PU.11 are also prepared.

### Example 4

Shampoo
Phase A

| | |
|---|---|
| 15.00 g | Cocamidopropyl Betaine |
| 10.00 g | Disodium Cocoamphodiacetate |
| 5.00 g | Polysorbate 20 |
| 5.00 g | Decyl Glucoside |
| 0.50 g | Polyquaternium-7, PQ-10, PQ-39, PQ-44, PQ-67, |
| | Guarhydroxypropyltrimonium Chloride and/or PQ-87 |
| 0.20 g | Polyurethane PU.1 |
| 0.10 g | Perfume oil/essential oil |
| q.s. | Preservative |
| 2.00 g | Laureth-3 |
| ad 100 | Aqua dem. |
| q.s. | Citric Acid |

Phase B

| | |
|---|---|
| 3.00 g | PEG-150 Distearate |

### Preparation

Weigh in the components of phase A and dissolve; adjust pH to 6-7. Add phase B and heat to 50° C. Allow to cool to room temperature with stirring.

Instead of the shampoo comprising the polyurethane PU.1, shampoos comprising one or more of the polyurethanes PU.2 to PU.11 are also prepared.

Shampoo

| | |
|---|---|
| 30.00 g | Sodium Laureth Sulfate |
| 6.00 g | Sodium Cocoamphoacetate |
| 0.50 g | Polyquaternium-7, PQ-10, PQ-39, PQ-44, PQ-67, |
| | Guarhydroxypropyltrimonium Chloride and/or PQ-87 |
| 0.50 g | Polyurethane PU.1 |
| 3.00 g | Sodium Laureth Sulfate, Glycol Distearate, Cocamide MEA, |
| | Laureth-10 |
| 2.00 g | Dimethicone |
| q.s. | Perfume |
| q.s. | Preservative |
| q.s. | Citric Acid |
| 1.00 g | Sodium Chloride |
| ad 100 | Aqua dem. |

Instead of the shampoo comprising the polyurethane PU.1, shampoos comprising one or more of the polyurethanes PU.2 to PU.11 are also prepared.

Shower gel 1

| | |
|---|---|
| 20.00 g | Ammonium Laureth Sulfate |
| 15.00 g | Ammonium Lauryl Sulfate |
| 0.50 g | Polyurethane PU.1 |
| 0.50 g | Polyquaternium-10, PQ-22, PQ-44, PQ-67, |
| | Guarhydroxypropyltrimonium Chloride |
| | and/or PQ-87 |
| 2.50 g | Sodium Laureth Sulfate, Glycol Distearate, |
| | Cocamide MEA, Laureth-10 |
| 0.10 g | Perfume oil/essential oil |
| q.s. | Preservative |
| 0.50 g | Sodium Chloride |
| ad 100 | Aqua dem. |

Instead of the shower gel 1 comprising the polyurethane PU.1, shower gels comprising one or more of the polyurethanes PU.2 to PU.11 are also prepared.

Shower gel 2

| | |
|---|---|
| 40.00 g | Sodium Laureth Sulfate |
| 5.00 g | Decyl Glucoside |
| 5.00 g | Polyurethane PU.1 |
| 1.00 g | Panthenol |
| 0.50 g | Polyquaternium-10, PQ-44, PQ-67, |
| | Guarhydroxypropyltrimonium Chloride |
| | and/or PQ-87 |
| 0.10 g | Perfume oil/essential oil |
| q.s. | Preservative |
| q.s. | Citric Acid |
| 2.00 g | Sodium Chloride |
| ad 100 | Aqua dem. |

Instead of shower gel 2 comprising the polyurethane PU.1, shower gels comprising one or more of the polyurethanes PU.2 to PU.11 are also prepared.

Shampoo

| | |
|---|---|
| 12.00 g | Sodium Laureth Sulfate |
| 1.50 g | Decyl Glucoside |
| 0.50 g | Polyquaternium-10, PQ-44, PQ-67, |
| | Guarhydroxypropyltrimonium Chloride |
| | and/or PQ-87 |
| 0.50 g | Polyurethane PU.1 |
| 5.00 g | Coco-Glucoside Glyceryl Oleate |
| 2.00 g | Sodium Laureth Sulfate, Glycol Distearate, |
| | Cocomide MEA, Laureth-10 |
| q.s. | Preservative |
| q.s. | Sunset Yellow C.I. 15 985 |
| 0.10 g | Perfume oil/essential oil |
| 1.00 g | Sodium Chloride |
| ad 100 | Aqua dem. |

Instead of this shampoo comprising the polyurethane PU.1, shampoos comprising one or more of the polyurethanes PU.2 to PU.11 are also prepared.

The polyurethanes PU can also be used in hair styling preparations, in particular hair foams (aerosol foams with propellant gas and pump foams without propellant gas), hair sprays (pump sprays without propellant gas) and hair gels.

Propellants are the customarily used propellants. Preference is given to mixtures of propane/butane, pentane, dimethyl ether, 1,1-difluoroethane (HFC-152a), carbon dioxide, nitrogen or compressed air.

Aerosol hair foam

| | |
|---|---|
| 2.00 g | Cocotrimonium Methosulfate |
| 0.10 g | Perfume oil/essential oil |
| 3.50 g | Setting polymer or combinations of e.g. PVP, |
| | PVP/VA copolymer, Polyquaternium-4, PQ-11, |
| | PQ-16, PQ-46, PQ-44, PQ-68, VP/ |
| | Methacrylamide/Vinyl Imidazole |
| | Copolymer, etc. |
| 0.80 g | Polyurethane PU.1 |
| q.s. | Preservative |
| 75.00 g | Water dem. |
| 10.00 g | Propane/Butane (3.5 bar) |

Instead of this aerosol hair foam comprising the polyurethane PU.1, aerosol hair foams comprising one or more of the polyurethanes PU.2 to PU.11 are also prepared.

Hairstyling gel 1
Phase A

| | |
|---|---|
| 0.50 g | Carbomer or Acrylates/C10-30 Alkyl |
| | Acrylate Crosspolymer |
| 86.40 g | Water dem. |

Phase B

| | |
|---|---|
| 0.70 g | Triethanolamine |

Phase C

| | |
|---|---|
| 11.00 g | Setting polymer or combinations of e.g. PVP, |
| | PVP/VA copolymer, Polyquaternium-4, PQ-11, |
| | PQ-16, PQ-46, PQ-44, PQ-68, |
| | VP/Methacrylamide/Vinyl Imidazole |
| | Copolymer, etc. |
| 0.20 g | PEG-25 PABA |
| 2.00 g | Polyurethane PU.1 |
| 0.10 g | Perfume oil/essential oil |
| q.s. | PEG-14 Dimethicone |
| q.s. | Preservative |
| 0.10 g | Tocopheryl acetate |

Instead of this hairstyling gel 1 comprising the polyurethane PU.1, hairstyling gels comprising one or more of the polyurethanes PU.2 to PU.11 are also prepared.

Hairstyling gel 2
Phase A

| | |
|---|---|
| 0.50 g | Carbomer or Acrylates/C10-30 Alkyl |
| | Acrylate Crosspolymer |
| 91.20 g | Water dem. |

Phase B

| | |
|---|---|
| 0.90 g | Tetrahydroxypropyl Ethylenediamine |

Phase C

| | |
|---|---|
| 7.00 g | VP/VA Copolymer |
| 0.70 g | Polyurethane PU.1 |
| 0.20 g | Perfume oil/essential oil |
| q.s. | Preservative |
| 0.10 g | Propylene Glycol |

Instead of this hairstyling gel 2 comprising the polyurethane PU.1, hairstyling gels comprising one or more of the polyurethanes PU.2 to PU.11 are also prepared.

Hair Wax Cream

| 6.00 | g | Caprylic/Capric Triglyceride |
|---|---|---|
| 3.00 | g | Glyceryl Stearate |
| 2.00 | g | Cetyl Alcohol |
| 3.50 | g | Polyurethane PU.1 |
| 0.50 | g | Cremophor A6 |
| 0.70 | g | Cremophor A25 |
| 0.50 | g | Dimethicone |
| 0.50 | g | Vitamin E Acetate |
| 2.00 | g | Caprylic/Capric Triglyceride and |
| | | Sodiumacrylates Copolymer |
| 1.00 | g | D-Panthenol USP |
| 0.10 | g | EDTA |
| 10.00 | g | Setting polymer |
| q.s. | | Preservative |
| ad 100 | g | Water dem. |

Instead of this hair wax cream comprising the polyurethane PU.1, hair wax creams comprising one or more of the polyurethanes PU.2 to PU.11 are also prepared.

Hair pudding

| 3.00 | g | Kollicoat IR (BASF) |
|---|---|---|
| q.s. | | Preservative |
| 2.00 | g | Setting polymer |
| 4.00 | g | Acrylates/beheneth-25 Methacrylate Copolymer |
| 0.70 | g | Polyurethane PU.1 |
| 0.50 | g | Dimethicone Copolyol |
| 0.10 | g | EDTA |
| 0.20 | g | Benzophenone-4 |
| ad 100 | g | Water dem. |

Instead of this hair pudding comprising the polyurethane PU.1, hair puddings comprising one or more of the polyurethanes PU.2 to PU.11 are also prepared.

Spray gel
Phase A

| | |
|---|---|
| 1.25 g | Setting polymer |
| 96.15 g | Aqua dem. |

Phase B

| | |
|---|---|
| 0.70 g | Acrylates/Steareth-20 Itaconate Copolymer |
| 0.10 g | Propylene Glycol |
| 0.50 g | Polyurethane PU.1 |
| 0.10 g | Glycerol |
| 0.10 g | Perfume oil/essential oil |
| q.s. | Preservative |

Phase C

| | |
|---|---|
| 0.70 g | Triethanolamine |

Instead of this spray gel comprising the polyurethane PU.1, spray gels comprising one or more of the polyurethanes PU.2 to PU.11 are also prepared.

A preparation suitable according to the invention for styling sprays can for example have the following composition:

Pump hairspray

| | |
|---|---|
| 11.20 g | PEG/PPG-25125 Dimethicone/Acrylates Copolymer or |
| | Acrylates Copolymer |
| 2.80 g | VP/VA Copolymer |
| 1.34 g | Aminomethyl Propanol |
| 0.30 g | Polyurethane PU.1 |
| 0.10 g | Perfume oil/essential oil |
| 11.26 g | Aqua dem. |
| 73.00 g | Alcohol |

Instead of this pump hairspray comprising the polyurethane PU.1, spray gels comprising one or more of the polyurethanes PU.2 to PU.11 are also prepared.

Pump hairspray VOC55

| | |
|---|---|
| 2.00 g | VP/Methacrylamide/Vinyl Imidazole Copolymer |
| 1.90 g | Polyquaternium-46 |
| 2.00 g | Polyurethane PU.1 |
| 0.10 g | Perfume oil/essential oil |
| 55.00 g | Alcohol |
| 39.00 g | Aqua dem. |

Decorative cosmetic compositions

Instead of this pump hairspray V0055 comprising the polyurethane PU.1, spray gels comprising one or more of the polyurethanes PU.2 to PU.11 are also prepared.

Liquid make-up
Phase A

| | |
|---|---|
| 1.70 g | Glyceryl Stearate |
| 1.70 g | Cetyl Alcohol |
| 1.70 g | Ceteareth-6 |
| 1.70 g | Ceteareth-25 |
| 5.20 g | Caprylic/Capric Triglyceride |
| 5.20 g | Mineral Oil or Luvitol ® Lite (INCl Hydrogenated |
| | Polyisobutene) |

Phase B

| | |
|---|---|
| q.s. | Preservative |
| 4.30 g | Propylene Glycol |
| 2.50 g | Polyurethane PU.1 |
| 59.50 g | Aqua dem. |

Phase C

| | |
|---|---|
| 0.10 g | Perfume oil/essential oil |

Phase D

| | |
|---|---|
| 2.00 g | Iron Oxides |
| 12.00 g | Titanium Dioxide |

Instead of this liquid make-up comprising the polyurethane PU.1, liquid make-ups comprising one or more of the polyurethanes PU.2 to PU.11 are also prepared.

Eyeliner
Phase A

| | |
|---|---|
| 40.60 g | dist. water |
| 0.20 g | Disodium EDTA |
| q.s. | Preservative |

Phase B

| | |
|---|---|
| 0.60 g | Xanthan Gum |
| 0.40 g | Veegum |
| 3.00 g | Butylene Glycol |
| 0.20 g | Polysorbate-20 |

Phase C

| | |
|---|---|
| 15.00 g | Iron oxide/Al Powder/Silica (e.g. Sicopearl ® Fantastico |
| | Gold from BASF or other effect pigments) |

Phase D

| | |
|---|---|
| 10.00 g | Aqua dem. |
| 25.00 g | Setting polymer or combinations of e.g. PVP, PVP/VA |
| | copolymer, Polyquaternium-4, PQ-11, PQ-16, PQ-46, PQ-44, |
| | PQ-68, Polyurethane-1 or VP/Methacrylamide/Vinyl |
| | Imidazole Copolymer, etc. |
| 5.00 g | Polyurethane PU.1 |

Instead of this eyeliner comprising the polyurethane PU.1, eyeliners comprising one or more of the polyurethanes PU.2 to PU.11 are also prepared.

Face toner
Phase A

| | |
|---|---|
| 3.00 g | Polyurethane PU.1 |
| 0.10 g | Perfume oil/essential oil |
| 0.30 g | Bisabolol |

Phase B

| | |
|---|---|
| 3.00 g | Glycerol |
| 1.00 g | Hydroxyethyl Cetyldimonium Phosphate |
| 5.00 g | Witch Hazel (Hamamelis Virginiana) Distillate |
| 0.50 g | Panthenol |
| q.s. | Preservative |
| 87.60 g | Aqua dem. |

Instead of this face toner comprising the polyurethane PU.1, face toners comprising one or more of the polyurethanes PU.2 to PU.11 are also prepared.

Face washing paste with peeling effect
Phase A

| | |
|---|---|
| 73.00 g | Aqua dem. |
| 1.50 g | Polyurethane PU.1 |
| q.s. | Preservative |

Phase B

| | |
|---|---|
| q.s. | Perfume oil |
| 7.00 g | Potassium Cocoyl Hydrolyzed Protein |
| 4.00 g | Conditioning polymer or combinations of Polyquaternium-7, |
| | PQ-10, PQ-39, PQ-44, PQ-67, Guarhydroxypropyltrimonium |
| | Chloride, PQ-87 |

Phase C

| | |
|---|---|
| 1.50 g | Triethanolamine |

Phase D

| | |
|---|---|
| 13.00 g | Polyethylene (Luwax A ™ from BASF) |

Instead of this face washing paste comprising the polyurethane PU.1, face washing pastes comprising one or more of the polyurethanes PU.2 to PU.11 are also prepared.

Soap
Phase A

| | |
|---|---|
| 25.00 g | Potassium Cocoate |
| 20.00 g | Disodium Cocoamphodiacetate |
| 2.00 g | Lauramide DEA |
| 1.0 g | Glycol Stearate |
| 2.00 g | Polyurethane PU.1 |
| 0.50 g | Conditioning polymer or combinations of Polyquaternium-7, |
| | PQ-10, PQ-39, PQ-44, PQ-67, Guarhydroxypropyltrimonium |
| | Chloride, PQ-87 |
| 50.00 g | Aqua dem. |
| q.s. | Citric Acid |

Phase B

| | |
|---|---|
| q.s. | Preservative |
| 0.10 g | Perfume oil/essential oil |

Instead of this soap comprising the polyurethane PU.1, soaps comprising one or more of the polyurethanes PU.2 to PU.11 are also prepared.

Face cleansing milk O/W type
Phase A

| | |
|---|---|
| 1.50 g | Ceteareth-6 |
| 1.50 g | Ceteareth-25 |
| 2.00 g | Glyceryl Stearate |
| 2.00 g | Cetyl Alcohol |
| 10.00 g | Mineral Oil |

Phase B

| | |
|---|---|
| 5.00 g | Propylene Glycol |
| q.s. | Preservative |
| 1.00 g | Conditioning polymer or combinations of Polyquaternium-7, |
| | PQ-10, PQ-39, PQ-44, PQ-67, Guarhydroxypropyltrimonium |
| | Chloride, PQ-87 |
| 66.30 g | Aqua dem. |

Phase C

| | |
|---|---|
| 0.20 g | Polyurethane PU.1 |
| 10.00 g | Cetearyl Octanoate |

Phase D

| | |
|---|---|
| 0.40 g | Tetrahydroxypropyl Ethylenediamine |

Phase E

| | |
|---|---|
| 0.10 g | Perfume oil/essential oil |
| 0.10 g | Bisabolol |

Instead of this face cleansing milk comprising the polyurethane PU.1, face cleansing milks comprising one or more of the polyurethanes PU.2 to PU.11 are also prepared.

Transparent soap

| | |
|---|---|
| 4.20 g | Sodium Hydroxide |
| 3.60 g | dist. water |
| 5.00 g | Conditioning polymer or combinations of Polyquaternium-7, |
| | PQ-10, PQ-39, PQ-44, PQ-67, Guarhydroxypropyltrimonium |
| | Chloride, PQ-87 |
| 5.00 g | Polyurethane PU.1 |
| 22.60 g | Propylene Glycol |
| 18.70 g | Glycerol |
| 5.20 g | Cocoamide DEA |
| 2.40 g | Cocamine Oxide |
| 4.20 g | Sodium Lauryl Sulfate |
| 7.30 g | Myristic Acid |
| 16.60 g | Stearic Acid |
| 5.20 g | Tocopherol |

Instead of this transparent soap comprising the polyurethane PU.1, transparent soaps comprising one or more of the polyurethanes PU.2 to PU.11 are also prepared.

Shaving foam

| | |
|---|---|
| 6.00 g | Ceteareth-25 |
| 5.00 g | Poloxamer 407 |
| 52.00 g | Aqua dem. |
| 1.00 g | Triethanolamine |
| 5.00 g | Propylene Glycol |
| 1.00 g | PEG-75 Lanolin Oil |
| 2.00 g | Conditioning polymer or combinations of Polyquaternium-7, |
| | PQ-10, PQ-39, PQ-44, PQ-67, Guarhydroxypropyltrimonium |
| | Chloride, PQ-87 |
| 3.00 g | Polyurethane PU.1 |
| q.s. | Preservative |
| 0.10 g | Perfume oil/essential oil |
| 25.00 g | Sodium Laureth Sulfate |

Bottling: 90 parts of active substance and 10 parts of propane/butane mixture 25:75.

Instead of this shaving foam comprising the polyurethane PU.1, shaving foams comprising one or more of the polyurethanes PU.2 to PU.11 are also prepared.

After Shave Balm
Phase A

| | |
|---|---|
| 0.25 g | Polyurethane PU.1 |
| 1.50 g | Tocopheryl Acetate |
| 0.20 g | Bisabolol |
| 10.00 g | Caprylic/Capric Triglyceride |
| q.s. | Perfume |
| 1.00 g | Conditioning polymer or combinations of Polyquaternium-7, |
| | PQ-10, PQ-39, PQ-44, PQ-67, Guarhydroxypropyltrimonium |
| | Chloride, PQ-87 |

Phase B

| | |
|---|---|
| 1.00 g | Panthenol |
| 15.00 g | Alcohol |
| 5.00 g | Glycerol |
| 0.05 g | Hydroxyethyl Cellulose |
| 1.90 g | Polyurethane PU.1 |
| 64.02 g | dist. water |

Phase C

| | |
|---|---|
| 0.08 g | Sodium Hydroxide |

Instead of this after shave balm comprising the polyurethane PU.1, after shave balms comprising one or more of the polyurethanes PU.2 to PU.11 are also prepared.

Toothpaste
Phase A

| | |
|---|---|
| 34.79 g | Aqua dem. |
| 3.00 g | Polyurethane PU.1 |
| 20.00 g | Glycerol |
| 0.76 g | Sodium Monofluorophosphate |

Phase B

| | |
|---|---|
| 1.20 g | Sodium Carboxymethylcellulose |

Phase C

| | |
|---|---|
| 0.80 g | Aroma oil |
| 0.06 g | Saccharin |
| q.s. | Preservative |
| 0.05 g | Bisabolol |
| 1.00 g | Panthenol |
| 0.50 g | Tocopheryl Acetate |
| 2.80 g | Silica |
| 1.00 g | Sodium Lauryl Sulfate |
| 7.90 g | Dicalciumphosphate Anhydrate |
| 25.29 g | Dicalciumphosphate Dihydrate |
| 0.45 g | Titanium Dioxide |

Instead of this toothpaste comprising the polyurethane PU.1, toothpastes comprising one or more of the polyurethanes PU.2 to PU.11 are also prepared.

Mouthwash
Phase A

| | |
|---|---|
| 2.00 g | Aroma oil |
| 4.50 g | Polyurethane PU.1 |
| 1.00 g | Bisabolol |
| 30.00 g | Alcohol |

Phase B

| | |
|---|---|
| 0.20 g | Saccharin |
| 5.00 g | Glycerol |
| q.s. | Preservative |
| 5.00 g | Poloxamer 407 |
| 52.30 g | Aqua dem. |

Instead of this mouthwash comprising the polyurethane PU.1, mouthwashes comprising one or more of the polyurethanes PU.2 to PU.11 are also prepared.

Prosthesis adhesive
Phase A

| | |
|---|---|
| 0.20 g | Bisabolol |
| 1.00 g | Beta-Carotene |
| q.s. | Aroma oil |
| 20.00 g | Cetearyl Octanoate |
| 5.00 g | Silica |
| 33.80 g | Mineral Oil |

Phase B

| | |
|---|---|
| 5.00 g | Polyurethane PU.1 |
| 35.00 g | PVP (20% strength solution in water) |

Instead of this prosthesis adhesive comprising the polyurethane PU.1, prosthesis adhesives comprising one or more of the polyurethanes PU.2 to PU.11 are also prepared.

1. A cosmetic preparation comprising a water-dispersible polyurethane (PU) with an essentially linear backbone composed of alternating hydrophilic and hydrophobic sections, where
a. the two terminal sections (T) are hydrophobic,
b. in each case one hydrophilic section (S) directly adjoins each section T,
c. at least one hydrophobic section (D) directly adjoins each section S on at least one side, and
d. where at least one hydrophilic section (P) is present, where at least one hydrophobic section D separates two sections P if more than one section P is present,
and the polyurethane comprises at least three hydrophilic sections, and the ratio of the molecular weights of each hydrophilic section S to the molecular weight of each hydrophilic section P is from 1:1.4 to 1:140, the at least two hydrophobic sections D are aliphatic diisocyanate radicals and the at least one hydrophilic section P is a polyether radical with a number-average molecular weight of at least 1500 g/mol.

2. The cosmetic preparation according to claim 1, where at least one of the two hydrophobic terminal sections T of the polyurethane is a branched alkyl radical.

3. The cosmetic preparation according to claim 1, where the polyurethane is a mixture of polyurethanes PU, the terminal, hydrophobic sections T of which are branched or unbranched alkyl radicals.

4. The cosmetic preparation according to claim 1, where all hydrophilic sections of the polyurethane are polyether radicals.

5. The cosmetic preparation according to claim 1, where the at least two hydrophilic sections S of the polyurethane are ethylene oxide radicals.

6. The cosmetic preparation according to claim 1, in which the at least one hydrophilic section P of the polyurethane has a number-average molecular weight of from 1500 to 10 000 g/mol.

7. The cosmetic preparation according to claim 1, where the cosmetic preparation comprises at least 0.5% by weight, of at least one salt and at least 0.1% by weight of at least one surfactant.

8. The cosmetic preparation according to claim 1, where the cosmetic preparation is of the oil-in-water emulsion type.

9. The cosmetic preparation according to claim 1, where the preparation is a photoprotective preparation and comprises zinc oxide and/or titanium dioxide as inorganic UV photoprotective filter.

10. The cosmetic preparation according to claim 1, where the cosmetic preparation comprises hydrogen peroxide.

11. The cosmetic preparation according to claim 1, where the cosmetic preparation comprises in the region of from 0.5 to 15% by weight of urea.

12. The cosmetic preparation according to claim 2, where all hydrophilic sections of the polyurethane are polyether radicals.

13. The cosmetic preparation according to claim 3, where all hydrophilic sections of the polyurethane are polyether radicals.

14. The cosmetic preparation according to claim 2, where the at least two hydrophilic sections S of the polyurethane are ethylene oxide radicals.

15. The cosmetic preparation according to claim 3, where the at least two hydrophilic sections S of the polyurethane are ethylene oxide radicals.

16. The cosmetic preparation according to claim 4, where the at least two hydrophilic sections S of the polyurethane are ethylene oxide radicals.

17. The cosmetic preparation according to claim 2, in which the at least one hydrophilic section P of the polyurethane has a number-average molecular weight of from 1500 to 10 000 g/mol.

18. The cosmetic preparation according to claim 3, in which the at least one hydrophilic section P of the polyurethane has a number-average molecular weight of from 1500 to 10 000 g/mol.

19. The cosmetic preparation according to claim 4, in which the at least one hydrophilic section P of the polyurethane has a number-average molecular weight of from 1500 to 10 000 g/mol.

20. The cosmetic preparation according to claim 5, in which the at least one hydrophilic section P of the polyurethane has a number-average molecular weight of from 1500 to 10 000 g/mol.
